(19) United States
(12) Patent Application Publication
(10) Pub. No.: US 2008/0287399 A1
(43) Pub. Date: Nov. 20, 2008

(54) Substituted Aminopyridines and Uses Thereof

(75) Inventors: Jeffrey Scott Albert, Wilmington, DE (US); Owen Callaghan, Den Bosch (NL); James Campbell, Wilmington, DE (US); Robin Arthur Ellis Carr, Bassingbourn (GB); Gianni Chessari, Cambridge (GB); Suzanna Cowan, Cambridge (GB); Miles Stuart Congreve, Cambridge (GB); Phil Edwards, Wilmington, DE (US); Martyn Frederickson, Cambridge (GB); Christopher William Murray, Cambridge (GB); Sahil Patel, Cambridge (GB)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/721,779

(22) PCT Filed: Dec. 12, 2005
(86) PCT No.: PCT/SE05/01891

§ 371 Date: Jan. 10, 2008

Related U.S. Application Data
(60) Provisional application No. 60/635,987, filed on Dec. 14, 2004.
(60) Provisional application No. 60/684,131, filed on May 24, 2005.

(51) Int. Cl. A61K 31/675; A61K 31/497; A61K 31/47; C07D 215/12; C07F 9/28; C07D 413/02; C07D 401/02; A61K 31/5377; A61P 25/28

(52) U.S. Cl. 514/89; 514/256; 514/311; 514/235.5; 544/333; 546/176; 544/131; 546/22

(57) ABSTRACT

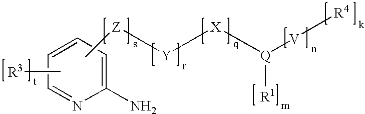

This invention relates to novel compounds having the structural formula (I) and to their pharmaceutically acceptable salt, compositions and methods of use. These novel compounds provide a treatment or prophylaxis of cognitive impairment, Alzheimer Disease, neurodegeneration and dementia.

### FIELD OF THE INVENTION

The present invention relates to novel substituted amino-pyridines, their pharmaceutical compositions, methods of use and processes to make such compounds. In addition, the present invention relates to therapeutic methods for the treatment and/or prevention of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, WO05/058311, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999)) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class I transmembrane protein called APP, or amyloid precursor protein. Aβ-secretase activity cleaves this protein between residues Met671 and Asp672 (numbering of 770aa isoform of APP) to form the N-terminus of Aβ. A second cleavage of the peptide is associated with γ-secretase to form the C-terminus of the Aβ peptide.

Alzheimer's disease (AD) is estimated to afflict more than 20 million people worldwide and is believed to be the most common form of dementia. Alzheimer's disease is a progressive dementia in which massive deposits of aggregated protein breakdown products amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much greater chance of developing AD, and also of developing it at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to acquire Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of APPβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No 6,211,235, WO02/02505, WO02/02506, WO02/02512, WO02/02518, WO02/02520, WO02/14264).

### SUMMARY OF THE INVENTION

Provided herein are novel compounds of structural formula (I) or a pharmaceutically acceptable salt thereof:

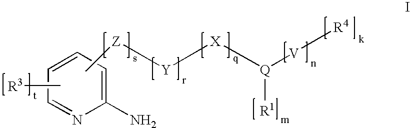

wherein:

Q is selected from C₃₋₁₂cycloalkyl, C₃₋₁₂cycloalkenyl, C₅₋₁₄aryl and C₅₋₁₄heterocyclyl;

R¹ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂;

R^{a} is selected from H, halogen, CN, NH₂, OH, C₁₋₆alkyl, OC₁₋₆alkyl, OC₁₋₆alkyl-OC₁₋₆alkyl, C(═O)C₁₋₆alkyl, C(═O)OC₁₋₆alkyl, OC(═O)C₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)_{2,—}NHC₃₋₁₂cycloalkyl, N(C₃₋₁₂cycloalkyl)₂, NHC(═O)C₃₋₁₂cycloalkyl, NC(═O)(C₃₋₁₂cycloalkyl)₂, C₅₋₆aryl, OC₅₋₆aryl, C(═O)C₅₋₆aryl, C(═O)OC₅₋₆aryl, C(═O)NH₂, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NHC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NH₂, C(═O)NHC₅₋₆heterocyclyl-, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NHC(═O)C₅₋₆heterocyclyl, and NC(═O)(C₅₋₆heterocyclyl)₂;

V is independently selected from N, O, S, SO, SO₂, NHSO₂, SO₂NH, NHC(═O), C(═O)NH, and optionally substituted C₁₋₆alkyl wherein such substituent is/are independently selected from R²;

X is selected from N, O, S, SO, SO₂, NHSO₂, SO₂NH, NHC(═O), C(═O)NH, and optionally substituted C₁₋₆alkyl, C₂₋₆alkenyl, or C₃₋₅cycloalkyl wherein such substituent is/are independently selected from R²;

Y is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;

Z is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;

k is 0, 1, 2, 3 or 4;

m is 0, 1, 2, 3 or 4;

n is 0, 1, 2, 3 or 4;

q is 0 or 1;

r is 0 or 1;

s is 0 or 1;

t is 0, 1 or 2;

where at least one of s, r or q are 1.

R² is independently selected from H, halogen, optionally substituted C₁₋₆alkyl, optionally substituted O—C₁₋₆alkyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, and NC(═O)(C5-6heterocyclyl)₂;

R³ is independently selected from H, halogen, CN, NH₂, OH, C₁₋₆alkyl-R^{a}, C₂₋₆alkenyl-R^{a}, C₂₋₆alkynyl-R^{a}, C₅₋₆cycloalkenyl-R^{a}, C₃₋₁₂cycloalkyl-R^{a}, C₁₋₆alkyl-C₃₋₁₂cycloalkyl-R^{a}, C₅₋₁₀aryl-R^{a}, C₁₋₆alkyl-C₅₋₁₀aryl-R^{a}, C₅₋₁₀heterocyclyl-R^{a}, C₁₋₆alkyl-C₅₋₁₀heterocyclyl-R^{a}, C(═O)C₅₋₆heterocyclyl-R^{a}, C(═O)C₅₋₁₀aryl-R^{a}, C(═O)C₃₋₁₂cycloalkyl-R^{a}, OC₁₋₆alkyl-R^{a}, O—C₅₋₁₀aryl-R^{a}, O—C₅₋₆heterocyclyl-R^{a}, O—C₃₋₁₂cycloalkyl-R^{a}, C(═O)OC₁₋₆allyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, NHC(═O)NHC₁₋₆alkyl, NHC(═O)OC₁₋₆alkyl, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHNH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O) (C₅₋₆aryl)₂, NHC(═O)NHC₅₋₆aryl, NHC(═O)OC₅₋₆aryl, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂, NHC(═O)NHC₅₋₆heterocyclyl, and NHC(═O)OC₅₋₆heterocyclyl;

R⁴ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C_{5 10}heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocycly)₂.

The invention also encompasses stereoisomers, enantiomers, and pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes.

### DETAILED DESCRIPTION OF THE INVENTION

Provided herein are novel compounds of structural formula (I) or a pharmaceutically acceptable salt, thereof:

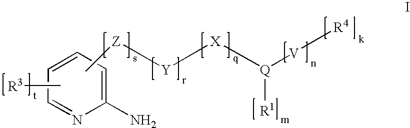

wherein:

Q is selected from C₃₋₁₂cycloalkyl, C₃₋₁₂cycloalkenyl, C₅₋₁₄aryl and C₅₋₁₄heterocyclyl;

R¹ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NEC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆Heterocyclyl-R^{a})₂, NHC(═O)C₅₋₆heterocyclyl, and NC(═O)(C₅₋₆heterocyclyl)₂;

R^{a} is selected from H, halogen, CN, NH₂, OH, C₁₋₆alkyl, OC₁₋₆alkyl, OC₁₋₆alkyl-OC₁₋₆alkyl, C(═O)C₁₋₆alkyl, C(═O)OC₁₋₆alkyl, OC(═O)C₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)_{2,—}NHC₃₋₁₂cycloalkyl, N(C₃₋₁₂cycloalkyl)₂, NHC(═O)C₃₋₁₂cycloalkyl, NC(═O)(C₃₋₁₂cycloalkyl)₂, C₅₋₆aryl, OC₅₋₆aryl, C(═O)C₅₋₆aryl, C(═O)OC₅₋₆aryl, C(═O)NH₂, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NHC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NH₂, C(═O)NHC₅₋₆heterocyclyl-, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NHC(═O)C₅₋₆heterocyclyl, and NC(═O)(C₅₋₆heterocyclyl)₂;

V is independently selected from N, O, S, SO, SO₂, NHSO₂, SO₂NH, NHC(═O), C(═O)NH, and optionally substituted C₁₋₆alkyl wherein such substituent is/are independently selected from R²;

X is selected from N, O, S, SO, SO₂, NHSO₂, SO₂NH, NHC(═O), C(═O)NH, and optionally substituted C₁₋₆alkyl, C₂₋₆alkenyl, or C₃₋₅cycloalkyl wherein such substituent is/are independently selected from R²;

Y is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;

Z is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;

k is 0, 1, 2, 3 or 4;

m is 0, 1, 2, 3 or 4;

n is 0, 1, 2, 3 or 4;

q is 0 or 1;

r is 0 or 1;

s is 0 or 1;

t is 0, 1 or 2;

where at least one of s, r or q are 1.

R² is independently selected from H, halogen, optionally substituted C₁₋₆alkyl, optionally substituted O—C₁₋₆alkyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, and NC(═O)(C₅₋₆heterocyclyl)₂;

R³ is independently selected from H, halogen, CN, NH₂, OH, C₁₋₆alkyl-R^{a}, C₂₋₆alkenyl-R^{a}, C₂₋₆alkynyl-R^{a}, C₅₋₆cycloalkenyl-R^{a}, C₃₋₁₂cycloalkyl-R^{a}, C₁₋₆alkyl-C₃₋₁₂cycloalkyl-R^{a}, C₅₋₁₀aryl-R^{a}, C₁₋₆alkyl-C₅₋₁₀aryl-R^{a}, C₅₋₁₀heterocyclyl-R^{a}, C₁₋₆alkyl-C₅₋₁₀heterocyclyl-R^{a}, C(═O)C₅₋₆heterocyclyl-R^{a}, C(═O)C₅₋₁₀aryl-R^{a}, C(═O)C₃₋₁₂cycloalkyl-R^{a}, OC₁₋₆alkyl-R^{a}, O—C₅₋₁₀aryl-R^{a}, O—C₅₋₆heterocyclyl-R^{a}, O—C₃₋₁₂cycloalkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₆alkyl-R^{a}, SO₂NHC₁₋₆alkyl-R^{a}, SO₂N(C₁₋₆alkyl)₂-R^{a}, NH(C₁₋₆alkyl)₂-R^{a}, NHC(═O)C₁₋₆alkyl, NC(═O)(C₁₋₆alkyl)₂, NHC(═O)NHC₁₋₆allyl, NHC(═O)OC₁₋₆alkyl, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHNH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, NHC(═O)NHC₅₋₆aryl, NHC(═O)OC₅₋₆aryl, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂, NHC(═O)NHC₅₋₆heterocyclyl, and NHC(═O)OC₅₋₆heterocyclyl;

R⁴ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alklynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C5-10heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂.

In a further embodiment, the compounds of the present invention are represented formula Ia:

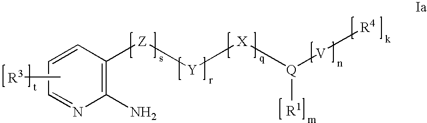

or a pharmaceutically acceptable salt thereof wherein:

Q is selected from C₃₋₁₂cycloalkyl, C₃₋₁₂cycloalkenyl, C₅₋₁₄aryl and C₅₋₁₄heterocyclyl;

R¹ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆al yl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(OC₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂;

R^{a} is selected from H, halogen, CN, NH₂, OH, C₁₋₆alkyl, OC₁₋₆alkyl, OC₁₋₆alkyl-OC₁₋₆alkyl, C(═O)C₁₋₆alkyl, C(═O)OC₁₋₆alkyl, OC(═O)C₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)_{2,—}NHC₃₋₁₂cycloalkyl, N(C₃₋₁₂cycloalkyl)₂, NHC(═O)C₃₋₁₂cycloalkyl, NC(═O)(C₃₋₁₂cycloalkyl)₂, C₅₋₆aryl, OC₅₋₆aryl, C(═O)C₅₋₆aryl, C(═O)OC₅₋₆aryl, C(═O)NH₂, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NHC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NH₂, C(═O)NHC₅₋₆heterocyclyl-, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NHC(═O)C₅₋₆heterocyclyl, and NC(═O)(C₅₋₆heterocyclyl)₂;

V is independently selected from N, O, S, SO, SO₂, NHSO₂, SO₂NH, NHC(═O), C(═O)NH, and optionally substituted C₁₋₆alkyl wherein such substituent is/are independently selected from R²;

X is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;

Y is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;

Z is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;

k is 0, 1, 2, 3 or 4;

m is 0, 1, 2, 3 or 4;

n is 0, 1, 2, 3 or 4;

q is 0 or 1;

r is 0 or 1;

s is 0 or 1;

t is 0, 1 or 2;

where at least one of s, r or q are 1.

R² is independently selected from H, halogen, optionally substituted C₁₋₆alkyl, optionally substituted O—C₁₋₆alkyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)₂-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, and NC(═O)(C₅₋₆heterocycly)₂;

R³ is independently selected from H, halogen, CN, NH₂, OH, C₁₋₆alkyl-R^{a}, C₂₋₆alkenyl-R^{a}, C₂₋₆alkynyl-R^{a}, C₅₋₆cycloalkenyl-R^{a}, C₃₋₁₂cycloalkyl-R^{a}, C₁₋₆alkyl-C₃₋₁₂cycloalkyl-R^{a}, C₅₋₁₀aryl-R^{a}, C₁₋₆alkyl-C₅₋₁₀aryl-R^{a}, C₅₋₁₀heterocyclyl-R^{a}, C₁₋₆alkyl-C₅₋₁₀heterocyclyl-R^{a}, C(═O)C₅₋₆heterocyclyl-R^{a}, C(═O)C₅₋₁₀aryl-R^{a}, C(═O)C₃₋₁₂cycloalkyl-R^{a}, OC₁₋₆alkyl-R^{a}, O—C₅₋₁₀aryl-R^{a}, O—C₅₋₆heterocyclyl-R^{a}, O—C₃₋₁₂cycloalkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, NHC(═O)NHC₁₋₆alkyl, NHC(═O)OC₁₋₆alkyl, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHNH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂, R^{a}, NH(C₅₋₆aryl)-R^{a}, N(C₅₋₆aryl)₂-R^{a}, NHC(═O)C₅₋₆aryl, NC(═O)(C₅₋₆aryl)₂, NHC(═O)NHC₅₋₆aryl, NHC(═O)OC₅₋₆aryl, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂, NHC(═O)NHC₅₋₆heterocyclyl, and NHC(═O)OC₅₋₆heterocyclyl;

R⁴ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NC(═O)(C₅₋₆heterocyclyl)₂.

In a further embodiment, the compounds of the present invention are represented formula Ib:

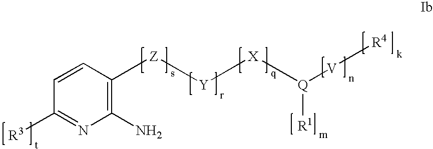

or a pharmaceutically acceptable salt thereof wherein:

Q is selected from C₃₋₁₂cycloalkyl, C₃₋₁₂cycloalkenyl, C₅₋₁₄aryl and C₅₋₁₄heterocyclyl;

R¹ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)OC₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocycyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂;

R^{a} is selected from H, halogen, CN, NH₂, OH, C₁₋₆alkyl, OC₁₋₆allyl, OC₁₋₆alkyl-OC₁₋₆alkyl, C(═O)C₁₋₆alkyl, C(═O)OC₁₋₆alkyl, OC(═O)C₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂,_NHC₃₋₁₂cycloalkyl, N(C₃₋₁₂ cycloalkyl)₂, NHC(═O)C₃₋₁₂cycloalkyl, NC(═O)(C₃₋₁₂cycloalkyl)₂, C₅₋₆aryl, OC₅₋₆aryl, C(═O)C₅₋₆aryl, C(═O)OC₅₋₆aryl, C(═O)NH₂, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NHC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NH₂, C(═O)NHC₅₋₆heterocyclyl-, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NHC(═O)C₅₋₆heterocyclyl, and NC(═O)(C₅₋₆heterocyclyl)₂;

V is independently selected from N, O, S, SO, SO₂, NHSO₂, SO₂NH, NHC(═O), C(═O)NH, and optionally substituted C₁₋₆alkyl wherein such substituent is/are independently selected from R²;

X is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;

Y is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;

Z is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;

k is 0, 1, 2, 3 or 4;

m is 0, 1, 2, 3 or 4;

n is 0, 1, 2, 3 or 4;

q is 0 or 1;

r is 0 or 1;

s is 0 or 1;

t is 0, or 1;

where at least one of s, r or q are 1.

R² is independently selected from H, halogen, optionally substituted C₁₋₆alkyl, optionally substituted O—C₁₋₆alkyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, and NC(═O)(C₅₋₆heterocyclyl)₂;

R³ is independently selected from H, halogen, CN, NH₂, OH, C₁₋₆alkyl-R^{a}, C₂₋₆alkenyl-R^{a}, C₂₋₆alkynyl-R^{a}, C₅₋₆cycloalkenyl-R^{a}, C₃₋₁₂cycloalkyl-R^{a}, C₁₋₆alkyl-C₃₋₁₂cycloalkyl-R^{a}, C₅₋₁₀aryl-R^{a}, C₁₋₆alkyl-C₅₋₁₀aryl-R^{a}, C₅₋₁₀heterocyclyl-R^{a}, C₁₋₆alkyl-C₅₋₁₀heterocyclyl-R^{a}, C(═O)C₅₋₆heterocyclyl-R^{a}, C(═O)C₅₋₁₀aryl-R^{a}, C(═O)C₃₋₁₂cycloalkyl-R^{a}, OC₁₋₆alkyl-R^{a}, O—C₅₋₁₀aryl-R^{a}, O—C₅₋₆heterocyclyl-R^{a}, O—C₃₋₁₂cycloalkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆allyl, NC(═O)(C₁₋₆alkyl)₂, NHC(═O)NHC₁₋₆alkyl, NHC(═O)OC₁₋₆alkyl, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHNH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, NHC(═O)NHC₅₋₆aryl, NHC(═O)OC₅₋₆aryl, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂, NHC(═O)NHC₅₋₆heterocyclyl, and NHC(═O)OC₅₋₆heterocyclyl;

R⁴ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂.

In a further embodiment, the compounds of the present invention are represented by formula Ic:

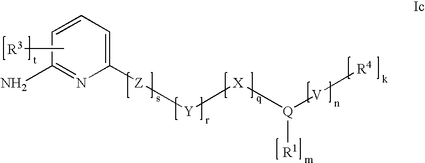

or a pharmaceutically acceptable salt thereof wherein:

Q is selected from C₃₋₁₂cycloalkyl, C₃₋₁₂cycloalkenyl, C₅₋₁₄aryl and C₅₋₁₄heterocyclyl;

R¹ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂;

R^{a} is selected from H, halogen, CN, NH₂, OH, C₁₋₆alkyl, OC₁₋₆alkyl, OC₁₋₆alkyl-OC₁₋₆alkyl, C(═O)C₁₋₆alkyl, C(═O)OC₁₋₆alkyl, OC(═O)C₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂,_NHC₃₋₁₂cycloalkyl, N(C₃₋₁₂cycloalkyl)₂, NHC(═O)C₃₋₁₂cycloalkyl, NC(═O)(C₃₋₁₂cycloalkyl)₂, C₅₋₆aryl, OC₅₋₆aryl, C(═O)C₅₋₆aryl, C(═O)OC₅₋₆aryl, C(═O)NH₂, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NHC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NH₂, C(═O)NHC₅₋₆heterocyclyl-, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NHC(═O)C₅₋₆heterocyclyl, and NC(═O)(C₅₋₆heterocyclyl)₂;

V is independently selected from N, O, S, SO, SO₂, NHSO₂, SO₂NH, NHC(═O), C(═O)NH, and optionally substituted C₁₋₆alkyl wherein such substituent is/are independently selected from R²;

X is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;

Y is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;

Z is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;

k is 0, 1, 2, 3 or 4;

m is 0, 1, 2, 3 or 4;

n is 0, 1, 2, 3 or 4;

q is 0 or 1;

r is 0 or 1;

s is 0 or 1;

t is 0, 1 or 2;

where at least one of s, r or q are 1.

R² is independently selected from H, halogen, optionally substituted C₁₋₆allyl, optionally substituted O—C₁₋₆alkyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)₂-R^{a}, SO₂C₅₋₆aryl R^{a}, SO₂NHC₅₋₆aryl R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, and NC(═O)(C₅₋₆heterocyclyl)₂;

R³ is independently selected from H, halogen, CN, NH₂, OH, C₁₋₆alkyl-R^{a}, C₂₋₆alkenyl-R^{a}, C₂₋₆alkynyl-R^{a}, C₅₋₆cycloalkenyl-R^{a}, C₃₋₁₂cycloalkyl-R^{a}, C₁₆alkyl-C₃₋₁₂cycloalkyl-R^{a}, C₅₋₁₀aryl-R^{a}, C₁₋₆alkyl-C₅₋₁₀aryl-R^{a}, C₅₋₁₀heterocyclyl-R^{a}, C₁₋₆alkyl-C₅₋₁₀heterocyclyl-R^{a}, C(═O)C₅₋₆heterocyclyl-R^{a}, C(═O)C₅₋₁₀aryl-R^{a}, C(═O)C₃₋₁₂cycloalkyl-R^{a}, OC₁₋₆alkyl-R^{a}, O—C₅₋₁₀aryl-R^{a}, O—C₅₋₆heterocyclyl-R^{a}, O—C₃₋₁₂cycloalkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, NHC(═O)NHC₁₋₆alkyl, NHC(═O)OC₁₋₆alkyl, C₅₋₆aryl-R^{a}, OC₅₋₆aryl-R^{a}, C(═O)C₅₋₆aryl-R^{a}, C(═O)OC₅₋₆aryl-R^{a}, C(═O)NHNH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, NHC(═O)NHC₅₋₆aryl, NHC(═O)OC₅₋₆aryl, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂, NHC(═O)NHC₅₋₆heterocyclyl, and NHC(═O)OC₅₋₆heterocyclyl;

R⁴ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆allyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a},SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂.

In a further embodiment, the compounds of the present invention are represented by formula (I) or a pharmaceutically salt thereof as recited herein wherein R¹, R^{a}, V, X, Y, Z, m, n, q, r, s, t, R², R³ and R⁴ have the meanings defined herein and Q is C₅₋₁₀aryl.

In a further embodiment, the compounds of the present invention are represented by formula (Ia) or a pharmaceutically salt thereof as recited herein wherein R¹, R^{a}, V, X, Y, Z, m, n, q, r, s, t, R², R³ and R⁴ have the meanings defined herein and Q is C₅₋₁₀aryl.

In a further embodiment, the compounds of the present invention are represented by formula (Ib) or a pharmaceutically salt thereof as recited herein wherein R¹, R^{a}, V, X, Y, Z, m, n, q, r, s, t, R², R³, and R⁴ have the meanings defined herein and Q is C₅₋₁₀aryl.

In a further embodiment, the compounds of the present invention are represented by formula (Ic) or a pharmaceutically salt thereof as recited herein wherein R¹, R^{a}, V, X, Y, Z, m, n, q, r, s, t, R², R³ and R⁴ have the meanings defined herein and Q is C₅₋₁₀aryl.

In a further embodiment, the compounds of the present invention are represented by formula (I) or a pharmaceutically salt thereof as recited herein wherein R¹, R^{a}, V, X, Y, Z, m, n, q, r, s, t, R², R³ and R⁴ have the meanings defined herein and Q is phenyl.

In a further embodiment, the compounds of the present invention are represented by formula (Ia) or a pharmaceutically salt thereof as recited herein wherein R¹, R^{a}, V, X, Y, Z, m, n, q, r, s, t, R², R³ and R⁴ have the meanings defined herein and Q is phenyl.

In a further embodiment, the compounds of the present invention are represented by formula (Ib) or a pharmaceutically salt thereof as recited herein wherein R¹, R^{a}, V, X, Y, Z, m, n, q, r, s, t, R², R³ and R⁴ have the meanings defined herein and Q is phenyl.

In a further embodiment, the compounds of the present invention are represented by formula (Ic) or a pharmaceutically salt thereof as recited herein wherein R¹, R^{a}, V, X, Y, Z, m, n, q, r, s, t, R², R³ and R⁴ have the meanings defined herein and Q is phenyl.

In a further embodiment, the compounds of the present invention are represented by formula (I) or a pharmaceutically salt thereof as recited herein wherein Q, V, X, Y, Z, m, n, q, r, s, t, R², R³ and R⁴ have the meanings defined herein and RI is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a} and wherein R^{a} is independently selected from H, CN, OH, and C₁₋₆alkyl.

In a further embodiment, the compounds of the present invention are represented by formula (Ia) or a pharmaceutically salt thereof as recited herein wherein Q, V, X, Y, Z, m, n, q, r, s, t, R², R³ and R⁴ have the meanings defined herein and R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a} and wherein R^{a} is independently selected from H, CN, OH, and C₁₋₆alkyl.

In a further embodiment, the compounds of the present invention are represented by formula (Ib) or a pharmaceutically salt thereof as recited herein wherein Q, V, X, Y, Z, m, n, q, r, s, t, R², R³ and R⁴ have the meanings defined herein and R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a} and wherein R^{a} is independently selected from H, CN, OH, and C₁₋₆alkyl.

In a further embodiment, the compounds of the present invention are represented by formula (Ic) or a pharmaceutically salt thereof as recited herein wherein Q, V, X, Y, Z, m, n, q, r, s, t, R², R³ and R⁴ have the meanings defined herein and R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a} and wherein R^{a} is independently selected from H, CN, OH, and C₁₋₆alkyl.

In a further embodiment, the compounds of the present invention are represented by formula (I) or a pharmaceutically salt thereof as recited herein wherein Q, R¹, R^{a}, X, Y, Z, m, n, q, r, s, t, R², R₃ and R⁴ have the meanings defined herein and V is O.

In a further embodiment, the compounds of the present invention are represented by formula (Ia) or a pharmaceutically salt thereof as recited herein wherein Q, R¹, R^{a}, X, Y, Z, m, n, q, r, s, t, R², R³ and R⁴ have the meanings defined herein and V is O.

In a further embodiment, the compounds of the present invention are represented by formula (Ib) or a pharmaceutically salt thereof as recited herein wherein Q, R¹, R^{a}, X, Y, Z, m, n, q, r, s, t, R¹, R³ and R⁴ have the meanings defined herein and V is O.

In a further embodiment, the compounds of the present invention are represented by formula (Ic) or a pharmaceutically salt thereof as recited herein wherein Q, R¹, R^{a}, X, Y, Z, m, n, q, r, s, t, R¹, R³ and R⁴ have the meanings defined herein and V is O.

In a further embodiment, the compounds of the present invention are represented by formula (I) or a pharmaceutically salt thereof as recited herein wherein Q, R¹, R^{a}, V, Y, Z, m, n, q, r, s, t, R², R³ and R⁴ have the meanings defined herein and X is C₁₋₆alkyl.

In a further embodiment, the compounds of the present invention are represented by formula (Ia) or a pharmaceutically salt thereof as recited herein wherein Q, R¹, R^{a}, V, Y, Z, m, n, q, r, s, t, R², R³ and R⁴ have the meanings defined herein and X is C₁₋₆alkyl.

In a further embodiment, the compounds of the present invention are represented by formula (Ib) or a pharmaceutically salt thereof as recited herein wherein Q, R¹, R^{a}, V, Y, Z, m, n, q, r, s, t, R², R³ and R⁴ have the meanings defined herein and X is C₁₋₆alkyl.

In a further embodiment, the compounds of the present invention are represented by formula (Ic) or a pharmaceutically salt thereof as recited herein wherein Q, R¹, R^{a}, V, Y, Z, m, n, q, r, s, t, R², R³ and R⁴ have the meanings defined herein and X is C₁₋₆alkyl.

In a further embodiment, the compounds of the present invention are represented by formula (I) or a pharmaceutically salt thereof as recited herein wherein Q, R¹, R^{a}, V, X, Z, m, n, q, r, s, t, R², R³ and R⁴ have the meanings defined herein and Y is C₁₋₆alkyl.

In a further embodiment, the compounds of the present invention are represented by formula (Ia) or a pharmaceutically salt thereof as recited herein wherein Q, R¹, R^{a}, V, X, Z, m, n, q, r, s, t, R², R³ and R⁴ have the meanings defined herein and Y is C₁₋₆alkyl.

In a further embodiment, the compounds of the present invention are represented by formula (Ib) or a pharmaceutically salt thereof as recited herein wherein Q, R¹, R^{a}, V, X, Z, m, n, q, r, s, t, R², R³ and R⁴ have the meanings defined herein and Y is C₁₋₆alkyl.

In a further embodiment, the compounds of the present invention are represented by formula (Ic) or a pharmaceutically salt thereof as recited herein wherein Q, R¹, R^{a}, V, X, Z, m, n, q, r, s, t, R², R³ and R⁴ have the meanings defined herein and Y is C₁₋₆alkyl.

In a further embodiment, the compounds of the present invention are represented by formula (I) or a pharmaceutically salt thereof as recited herein wherein Q, R¹, R^{a}, V, X, Y, m, n, q, r, s, t, R², R³ and R⁴ have the meanings defined herein and Z is N or C₁₋₆alkyl.

In a further embodiment, the compounds of the present invention are represented by formula (Ia) or a pharmaceutically salt thereof as recited herein wherein Q, R¹, R^{a}, V, X, Y, m, n, q, r, s, t, R², R³ and R⁴ have the meanings defined herein and Z is N or C₁₋₆alkyl.

In a further embodiment, the compounds of the present invention are represented by formula (Ib) or a pharmaceutically salt thereof as recited herein wherein Q, R¹, R^{a}, V, X, Y, m, n, q, r, s, t, R², R³ and R⁴ have the meanings defined herein and Z is N or C₁₋₆alkyl.

In a further embodiment, the compounds of the present invention are represented by formula (Ic) or a pharmaceutically salt thereof as recited herein wherein Q, R¹, R^{a}, V, X, Y, m, n, q, r, s, t, R², R³ and R⁴ have the meanings defined herein and Z is N or C₁₋₆alkyl.

In a further embodiment, the compounds of the present invention are represented by formula (I) or a pharmaceutically salt thereof as recited herein wherein Q, R¹, R^{a}, V, X, Y, Z, n, q, r, s, t, R², R³ and R⁴ have the meanings defined herein and k is 0, 1, or 2.

In a further embodiment, the compounds of the present invention are represented by formula (Ia) or a pharmaceutically salt thereof as recited herein wherein Q, R¹, R^{a}, V, X, Y, Z, n, q, r, s, t, R², R³ and R⁴ have the meanings defined herein and k is 0, 1, or 2.

In a further embodiment, the compounds of the present invention are represented by formula (Ib) or a pharmaceutically salt thereof as recited herein wherein Q, R¹, R^{a}, V, X, Y, Z, n, q, r, s, t, R², R³ and R⁴ have the meanings defined herein and k is 0, 1, or 2.

In a further embodiment, the compounds of the present invention are represented by formula (Ic) or a pharmaceutically salt thereof as recited herein wherein Q, R¹, R^{a}, V, X, Y, Z, n, q, r, s, t, R², R³ and R⁴ have the meanings defined herein and k is 0, 1, or 2.

In a further embodiment, the compounds of the present invention are represented by formula (I) or a pharmaceutically salt thereof as recited herein wherein Q, R¹, R^{a}, V, X, Y, Z, n, q, r, s, t, R², R³ and R⁴ have the meanings defined herein and m is 0, 1, or 2.

In a further embodiment, the compounds of the present invention are represented by formula (Ia) or a pharmaceutically salt thereof as recited herein wherein Q, R¹, R^{a}, V, X, Y, Z, n, q, r, s, t, R², R³ and R⁴ have the meanings defined herein and m is 0, 1, or 2.

In a further embodiment, the compounds of the present invention are represented by formula (Ib) or a pharmaceutically salt thereof as recited herein wherein Q, R¹, R^{a}, V, X, Y, Z, n, q, r, s, t, R², R³ and R⁴ have the meanings defined herein and m is 0, 1, or 2.

In a further embodiment, the compounds of the present invention are represented by formula (Ic) or a pharmaceutically salt thereof as recited in claim 3 wherein Q, R¹, R^{a}, V, X, Y, Z, n, q, r, s, t, R², R³ and R⁴ have the meanings defined in claim 3 and m is 0, 1, or 2.

In a further embodiment, the compounds of the present invention are represented by formula (I) or a pharmaceutically salt thereof as recited in claim 1 wherein Q, R¹, R^{a}, V, X, Y, Z, m, q, r, s, t, R², R³ and R⁴ have the meanings defined in claim 1 and n is 0, 1, or 2.

In a further embodiment, the compounds of the present invention are represented by formula (Ia) or a pharmaceutically salt thereof as recited in claim 2 wherein Q, R¹, R^{a}, V, X, Y, Z, m, q, r, s, t, R², R³ and R⁴ have the meanings defined in claim 2 and n is 0, 1, or 2.

In a further embodiment, the compounds of the present invention are represented by formula (Ib) or a pharmaceutically salt thereof as recited in claim 3 wherein Q, R¹, R^{a}, V, X, Y, Z, m, q, r, s, t, R², R³ and R⁴ have the meanings defined in claim 3 and n is 0, 1, or 2.

In a further embodiment, the compounds of the present invention are represented by formula (Ic) or a pharmaceutically salt thereof as recited in claim 4 wherein Q, R¹, R^{a}, V, X, Y, Z, m, q, r, s, t, R², R³ and R⁴ have the meanings defined in claim 4 and n is 0, 1,or 2.

In a further embodiment, the compounds of the present invention are represented by formula (I) or a pharmaceutically salt thereof as recited in claim 1 wherein Q, R¹, R^{a}, V, X, Y, Z, m, n, r, s, t, R², R³ and R⁴ have the meanings defined in claim 1 and q is 0.

In a further embodiment, the compounds of the present invention are represented by formula (Ia) or a pharmaceutically salt thereof as recited in claim 2 wherein Q, R¹, R^{a}, V, is X, Y, Z, m, n, r, s, t, R², R³ and R⁴ have the meanings defined in claim 2 and q is 0.

In a further embodiment, the compounds of the present invention are represented by formula (Ib) or a pharmaceutically salt thereof as recited in claim 3 wherein Q, R¹, R^{a}, V, X, Y, Z, m, n, r, s, t, R², R³ and R⁴ have the meanings defined in claim 3 and q is 0.

In a further embodiment, the compounds of the present invention are represented by formula (Ic) or a pharmaceutically salt thereof as recited in claim 4 wherein Q, R¹, R^{a}, V, X, Y, Z, m, n, r, s, t, R², R³ and R⁴ have the meanings defined in claim 4 and q is 0.

In a further embodiment, the compounds of the present invention are represented by formula (I) or a pharmaceutically salt thereof as recited in claim 1 wherein Q, R¹, R^{a}, V, X, Y, Z, m, n, q, s, t, R², R³ and R⁴ have the meanings defined in claim 1 and r is 1.

In a further embodiment, the compounds of the present invention are represented by formula (Ia) or a pharmaceutically salt thereof as recited in claim 2 wherein Q, R¹, R^{a}, V, X, Y, Z, m, n, q, s, t, R², R³ and R⁴ have the meanings defined in claim 2 and r is 1.

In a further embodiment, the compounds of the present invention are represented by formula (Ib) or a pharmaceutically salt thereof as recited in claim 3 wherein Q, R¹, R^{a}, V, X, Y, Z, m, n, q, s, t, R², R³ and R⁴ have the meanings defined in claim 3 and r is 1.

In a further embodiment, the compounds of the present invention are represented by formula (Ic) or a pharmaceutically salt thereof as recited in claim 4 wherein Q, R¹, R^{a}, V, X, Y, Z, m, n, q, s, t, R², R³ and R⁴ have the meanings defined in claim 4 and r is 1.

In a further embodiment, the compounds of the present invention are represented by formula (I) or a pharmaceutically salt thereof as recited in claim 1 wherein Q, R¹, R^{a}, V, X, Y, Z, m, n, q, t, R², R³ and R⁴ have the meanings defined in claim 1 and s is 1.

In a further embodiment, the compounds of the present invention are represented by formula (Ia) or a pharmaceutically salt thereof as recited in claim 2 wherein Q, R¹, R^{a}, V, X, Y, Z, m, n, q, t, R², R³ and R⁴ have the meanings defined in claim 2 and s is 1.

In a further embodiment, the compounds of the present invention are represented by formula (Ib) or a pharmaceutically salt thereof as recited in claim 3 wherein Q, R¹, R^{a}, V, X, Y, Z, m, n, q, t, R², R³ and R⁴ have the meanings defined in claim 3 and s is 1.

In a further embodiment, the compounds of the present invention are represented by formula (Ic) or a pharmaceutically salt thereof as recited in claim 4 wherein Q, R¹, R^{a}, V, X, Y, Z, m, n, q, t, R², R³ and R⁴ have the meanings defined in claim 4 and s is 1.

In a further embodiment, the compounds of the present invention are represented by formula (I) or a pharmaceutically salt thereof as recited in claim 1 wherein Q, R¹, R^{a}, V, X, Y, Z, m, n, q, r, s, t, R³ and R⁴ have the meanings defined in claim 1 and R² is independently selected from H, or C₁₋₆alkyl.

In a further embodiment, the compounds of the present invention are represented by formula (Ia) or a pharmaceutically salt thereof as recited in claim 2 wherein Q, R¹, R^{a}, V, X, Y, Z, m, n, q, r, s, t, R³ and R⁴ have the meanings defined in claim 2 and R² is independently selected from H, or C₁₋₆alkyl.

In a further embodiment, the compounds of the present invention are represented by formula (Ib) or a pharmaceutically salt thereof as recited in claim 3 wherein Q, R¹, R^{a}, V, X, Y, Z, m, n, q, r, s, t, R³ and R⁴ have the meanings defined in claim 3 and R² is independently selected from H, or C₁₋₆alkyl.

In a further embodiment, the compounds of the present invention are represented by formula (Ic) or a pharmaceutically salt thereof as recited in claim 4 wherein Q, R¹, R^{a}, V, X, Y, Z, m, n, q, r, s, t, R³ and R⁴ have the meanings defined in claim 4 and R² is independently selected from H, or C₁₋₆alkyl.

In a further embodiment, the compounds of the present invention are represented by formula (I) or a pharmaceutically salt thereof as recited in claim 1 wherein Q, R¹, R^{a}, V, X, Y, Z, m, n, q, r, s, t, R² and R⁴ have the meanings defined in claim 1 and R³ is independently selected from H, halogen, C₁₋₆alkyl-R^{a} wherein R^{a} is independently selected from H, CN, OH, or C₁₋₆alkyl.

In a further embodiment, the compounds of the present invention are represented by formula (Ia) or a pharmaceutically salt thereof as recited in claim 2 wherein Q, R¹, R^{a}, V, X, Y, Z, m, n, q, r, s, t, R² and R⁴ have the meanings defined in claim 2 and R³ is independently selected from H, halogen, C₁₋₆alkyl-R^{a} wherein R^{a} is independently selected from H, CN, OH, or C₁₋₆alkyl.

In a further embodiment, the compounds of the present invention are represented by formula (Ib) or a pharmaceutically salt thereof as recited in claim 3 wherein Q, R¹, R^{a}, V, X, Y, Z, m, n, q, r, s, t, R² and R⁴ have the meanings defined in claim 3 and R³ is independently selected from H, halogen, C₁₋₆alkyl-R^{a} wherein R^{a} is independently selected from H, CN, OH, or C₁₋₆alkyl.

In a further embodiment, the compounds of the present invention are represented by formula (Ic) or a pharmaceutically salt thereof as recited in claim 4 wherein Q, R¹, R^{a}, V, X, Y, Z, m, n, q, r, s, t, R² and R⁴ have the meanings defined in claim 4 and R³ is independently selected from H, halogen, C₁₋₆alkyl-R^{a} wherein R^{a} is independently selected from H, CN, OH, or C₁₋₆alkyl.

In a further embodiment, the compounds of the present invention are represented by formula (I) or a pharmaceutically salt thereof as recited in claim 1 wherein Q, V, X, Y, Z, m, n, q, r, s, t, R² and R³ have the meanings defined in claim 1 and R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, alkyl-R^{a}, and C₅₋₆heterocyclyl-R^{a} and wherein R^{a} is independently selected from H, CN, OH, and C₁₋₆alkyl.

In a further embodiment, the compounds of the present invention are represented by formula (Ia) or a pharmaceutically salt thereof as recited in claim 2 wherein Q, V, X, Y, Z, m, n, q, r, s, t, R² and R³ have the meanings defined in claim 2 and R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a} and C₅₋₆heterocyclyl-R^{a} and wherein R^{a} is independently selected from H, CN, OH, and C₁₋₆alkyl.

In a further embodiment, the compounds of the present invention are represented by formula (Ib) or a pharmaceutically salt thereof as recited in claim 3 wherein Q, V, X, Y, Z, m, n, q, r, s, t, R² and R³ have the meanings defined in claim 3 and R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a} and wherein R^{a} is independently selected from H, CN, OH, and C₁₋₆alkyl.

In a further embodiment, the compounds of the present invention are represented by formula (Ic) or a pharmaceutically salt thereof as recited in claim 4 wherein Q, V, X, Y, Z, m, n, q, r, s, t, R² and R³ have the meanings defined in claim 4 and R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a} and wherein R^{a} is independently selected from H, CN, OH, and C₁₋₆alkyl.

In a further embodiment, the compounds of the present invention are represented by formula (I) or a pharmaceutically acceptable salt, thereof

wherein:

Q is selected from C₅₋₁₄aryl or C₅₋₁₄heterocyclyl;

R¹ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂;

R^{a} is selected from H, halogen, CN, NH₂, OH, C₁₋₆alkyl, OC₁₋₆alkyl, OC₁₋₆alkyl-OC₁₋₆alkyl, C(═O)OC₁₋₆alkyl, and OC(═O)C₁₋₆alkyl;

V is O;

X is selected from N, O, S, SO, SO₂, NHSO₂, SO₂NH, NHC(═O), C(═O)NH, and optionally substituted C₁₋₆alkyl, C₂₋₆alkenyl or C₃cycloalkyl wherein such substituent is/are independently selected from R²;

Y is optionally substituted C₁₋₆alkyl wherein such substituent is/are independently selected from R²;

Z is selected from N, and optionally substituted C₁₋₆alkyl wherein such substituent is/are independently selected from R²;

k is 0, 1, 2, or 3;

m is 0, 1, 2, or 3;

n is 0, 1, or 2;

q is 0;

r is 1;

s is 1;

t is 0, 1 or 2

R² is independently selected from H, and optionally substituted C₁₋₆alkyl;

R³ is independently selected from H, halogen, CN, C₁₋₆alkyl-R^{a}, and C(═O)C₅₋₆heterocyclyl-R^{a}.

R⁴ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂.

In a further embodiment, the compounds of the present invention are represented by formula (Ia) or a pharmaceutically acceptable salt, thereof

wherein:

Q is selected from C₅₋₁₄aryl or C₅₋₁₄heterocyclyl;

R¹ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂;

R^{a} is selected from H, halogen, CN, NH₂, OH, C₁₋₆alkyl, OC₁₋₆alkyl, OC₁₋₆alkyl-OC₁₋₆alkyl, C(═O)OC₁₋₆alkyl, and OC(═O)C₁₋₆alkyl;

V is O;

X is selected from N, O, S, SO, SO₂, NHSO₂, SO₂NH, NHC(═O), C(═O)NH, and optionally substituted C₁₋₆alkyl, C₂₋₆alkenyl or C₃cycloalkyl wherein such substituent is/are independently selected from R²;

Y is optionally substituted C₁₋₆alkyl wherein such substituent is/are independently selected from R²;

Z is selected from N, and optionally substituted C₁₋₆alkyl wherein such substituent is/are independently selected from R²;

k is 0, 1, 2, or 3;

m is 0, 1, 2,or 3;

n is 0, 1, or 2;

q is 0;

r is 1;

s is 1;

t is 0, 1 or 2

R² is independently selected from H, and optionally substituted C₁₋₆alkyl;

R³ is independently selected from H, halogen, CN, C₁₋₆alkyl-R^{a}, and C(═O)C₅₋₆heterocyclyl-R^{a}.

R⁴ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂.

In a further embodiment, the compounds of the present invention are represented by formula (Ib) or a pharmaceutically acceptable salt, thereof

wherein:

Q is selected from C₅₋₁₄aryl or C₅₋₁₄heterocyclyl;

R¹ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂;

R^{a} is selected from H, halogen, CN, NH₂, OH, C₁₋₆alkyl, OC₁₋₆alkyl, OC₁₋₆alkyl-OC₁₋₆alkyl, C(═O)OC₁₋₆alkyl, and OC(═O)C₁₋₆alkyl;

V is O;

X is selected from N, O, S, SO, SO₂, NHSO₂, SO₂NH, NHC(═O), C(═O)NH, and optionally substituted C₁₋₆alkyl, C₂₋₆alkenyl or C₃cycloalkyl wherein such substituent is/are independently selected from R²;

Y is optionally substituted C₁₋₆alkyl wherein such substituent is/are independently selected from R²;

Z is selected from N, and optionally substituted C₁₋₆alkyl wherein such substituent is/are independently selected from R²;

k is 0, 1, 2, or 3;

m is 0, 1, 2, or 3;

n is 0, 1, or 2;

q is 0;

r is 1;

s is 1;

t is 0, or 1;

R² is independently selected from H, and optionally substituted C₁₋₆alkyl;

R³ is independently selected from H, halogen, CN, C₁₋₆alkyl-R^{a}, and C(═O)C₅₋₆heterocyclyl-R^{a}.

R⁴ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂.

In a further embodiment, the compounds of the present invention are represented by formula (Ic) or a pharmaceutically acceptable salt, thereof

wherein:

Q is selected from C₅₋₁₄aryl or C₅₋₁₄heterocyclyl;

R¹ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)OC₅₋₆aryl-R^{a}, C(═O)NH₂, C(═O)NHC₅₋₆aryl R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂;

R^{a} is selected from H, halogen, CN, NH₂, OH, C₁₋₆alkyl, OC₁₋₆alkyl, OC₁₋₆alkyl-OC₁₋₆alkyl, C(═O)OC₁₋₆alkyl, and OC(═O)C₁₋₆alkyl;

V is O;

X is selected from N, O, S, SO, SO₂, NHSO₂, SO₂NH, NHC(═O), C(═O)NH, and optionally substituted C₁₋₆alkyl, C₂₋₆alkenyl or C₃cycloalkyl wherein such substituent is/are independently selected from R²;

Y is optionally substituted C₁₋₆alkyl wherein such substituent is/are independently selected from R²;

Z is selected from N, and optionally substituted C₁₋₆alkyl wherein such substituent is/are independently selected from R²;

k is 0, 1, 2, or 3;

m is 0, 1, 2, or 3;

n is 0, 1, or 2;

q is 0;

r is 1;

s is 1;

t is 0, 1 or 2

R² is independently selected from H, and optionally substituted C₁₋₆alkyl;

R³ is independently selected from H, halogen, CN, C₁₋₆alkyl-R^{a}, and C(═O)C₅₋₆heterocyclyl-R^{a}.

R⁴ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{, C(═O)N(C}₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂.

In a further embodiment, the compounds of the present invention are represented by formula (I) or a pharmaceutically acceptable salt, thereof

wherein:

Q is C₅₋₁₄aryl;

R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};

R^{a} is selected from H, CN, OH, and C₁₋₆alkyl;

V is O;

Y is unsubstituted C₁₋₆alkyl;

Z is selected from N, and unsubstituted C₁₋₆alkyl;

k is 0, 1, or 2;

m is 0, 1, or 2;

n is 0, 1, or 2;

q is 0;

r is 1;

s is 1;

t is 0 or 1;

R³ is independently selected from H, halogen, or C₁₋₆alkyl-R^{a};

R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

In a further embodiment, the compounds of the present invention are represented by formula (Ia) or a pharmaceutically acceptable salt, thereof

wherein:

Q is C₅₋₁₄aryl;

R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆allyl-R^{a}, OC₁₋₆alkyl-R^{a}, C(═O)OC₁₋₆alkyl-R^{a}, and C₅₋₆heterocyclyl-R^{a};

R^{a} is selected from H, CN, OH, and C₁₋₆alkyl;

V is O;

Y is unsubstituted C₁₋₆alkyl;

Z is selected from N, and unsubstituted C₁₋₆alkyl;

k is 0, 1, or 2;

m is 0, 1, or 2;

n is 0, 1, or 2;

q is 0;

r is 1;

s is 1;

t is 0 or 1;

R³ is independently selected from H, halogen, or C₁₋₆alkyl-R^{a};

R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

In a further embodiment, the compounds of the present invention are represented by formula (Ib) or a pharmaceutically acceptable salt, thereof

wherein:

Q is C₅₋₁₄aryl;

R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};

R^{a} is selected from H, CN, OH, and C₁₋₆alkyl;

V is O;

Y is unsubstituted C₁₋₆alkyl;

Z is selected from N, and unsubstituted C₁₋₆alkyl;

k is 0, 1, or 2;

m is 0, 1, or 2;

n is 0, 1, or 2;

q is 0;

r is 1;

s is 1;

t is 0 or 1;

R³ is independently selected from H, halogen, or C₁₋₆alkyl-R^{a};

R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

In a further embodiment, the compounds of the present invention are represented by formula (Ic) or a pharmaceutically acceptable salt, thereof

wherein:

Q is C₅₋₁₄aryl;

R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};

R^{a} is selected from H, CN, OH, and C₁₋₆alkyl;

V is O;

Y is unsubstituted C₁₋₆alkyl;

Z is selected from N, and unsubstituted C₁₋₆alkyl;

k is 0, 1, or 2;

m is 0, 1, or 2;

n is 0, 1, or 2;

q is 0;

r is 1;

s is 1;

t is 0 or 1;

R³ is independently selected from H, halogen, or C₁₋₆alkyl-R^{a};

R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

In a further embodiment, the compounds of the present invention are represented by formula (I) or a pharmaceutically acceptable salt, thereof

wherein:

Q is C₅₋₁₀aryl;

R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};

R^{a} is selected from H, CN, OH, and C₁₋₆alkyl;

V is O;

Y is unsubstituted C₁₋₆alkyl;

Z is selected from N, and unsubstituted C₁₋₆alkyl;

k is 0, 1, or 2;

m is 0, 1, or 2;

n is 0, 1, or 2;

q is 0;

r is 1;

s is 1;

t is 0 or 1;

R³ is independently selected from H, halogen, or C₁₋₆alkyl-R^{a};

R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

In a further embodiment, the compounds of the present invention are represented by formula (Ia) or a pharmaceutically acceptable salt, thereof

wherein:

Q is C₅₋₁₀aryl;

R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};

R^{a} is selected from H, CN, OH, and C₁₋₆alkyl;

V is O;

Y is unsubstituted C₁₋₆alkyl;

Z is selected from N, and unsubstituted C₁₋₆alkyl;

k is 0, 1, or 2;

m is 0, 1, or 2;

n is 0, 1, or 2;

q is 0;

r is 1;

s is 1;

t is 0 or 1;

R³ is independently selected from H, halogen, or C₁₋₆alkyl-R^{a};

R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

In a further embodiment, the compounds of the present invention are represented by formula (Ib) or a pharmaceutically acceptable salt, thereof

wherein:

Q is C₅₋₁₀aryl;

R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};

R^{a} is selected from H, CN, OH, and C₁₋₆alkyl;

V is O;

Y is unsubstituted C₁₋₆alkyl;

Z is selected from N, and unsubstituted C₁₋₆alkyl;

k is 0, 1, or 2;

m is 0, 1, or 2;

n is 0, 1, or 2;

q is 0;

r is 1;

s is 1;

t is 0 or 1;

R³ is independently selected from H, halogen, or C₁₋₆alkyl-R^{a};

R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

In a further embodiment, the compounds of the present invention are represented by formula (Ic) or a pharmaceutically acceptable salt, thereof

wherein:

Q is C₅₋₁₀aryl;

R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};

R^{a} is selected from H, CN, OH, and C₁₋₆alkyl;

V is O;

Y is unsubstituted C₁₋₆alkyl;

Z is selected from N, and unsubstituted C₁₋₆alkyl;

k is 0, 1, or 2;

m is 0, 1, or 2;

n is 0, 1, or 2;

q is 0;

r is 1;

s is 1;

t is 0 or 1;

R³ is independently selected from H, halogen, or C₁₋₆alkyl-R^{a};

R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

In a further embodiment, the compounds of the present invention are represented by formula (I) or a pharmaceutically acceptable salt, thereof

wherein:

Q is phenyl;

R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};

R^{a} is selected from H, CN, OH, and C₁₋₆alkyl;

V is O;

Y is unsubstituted C₁₋₆alkyl;

Z is selected from N, and unsubstituted C₁₋₆alkyl;

m is 0, 1, or 2;

n is 0, 1, or 2;

q is 0;

r is 1;

s is 1;

t is 0 or 1;

R³ is independently selected from H, halogen, or C₁₋₆alkyl-R^{a}.

R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

In a further embodiment, the compounds of the present invention are represented by formula (Ia) or a pharmaceutically acceptable salt, thereof

wherein:

Q is phenyl;

R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};

R^{a} is selected from H, CN, OH, and C₁₋₆alkyl;

V is O;

Y is unsubstituted C₁₋₆alkyl;

Z is selected from N, and unsubstituted C₁₋₆alkyl;

m is 0, 1, or 2;

n is 0, 1, or 2;

q is 0;

r is 1;

s is 1;

t is 0 or 1;

R³ is independently selected from H, halogen, or C₁₋₆alkyl-R^{a}.

R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

In a further embodiment, the compounds of the present invention are represented by formula (Ib) or a pharmaceutically acceptable salt, thereof

wherein:

Q is phenyl;

R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};

R^{a} is selected from H, CN, OH, and C₁₋₆alkyl;

V is O;

Y is unsubstituted C₁₋₆alkyl;

Z is selected from N, and unsubstituted C₁₋₆alkyl;

m is 0, 1, or 2;

n is 0, 1, or 2;

q is 0;

r is 1;

s is 1;

t is 0 or 1;

R³ is independently selected from H, halogen, or C₁₋₆alkyl-R^{a}.

R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};

In a further embodiment, the compounds of the present invention are represented by formula (Ib) or a pharmaceutically acceptable salt, thereof

wherein:

Q is phenyl;

R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};

R^{a} is selected from H, CN, OH, and C₁₋₆alkyl;

V is O;

Y is unsubstituted C₁₋₆alkyl;

Z is selected from N, and unsubstituted C₁₋₆alkyl;

m is 0, 1, or 2;

n is 0, 1, or 2;

q is 0;

r is 1;

s is 1;

t is 0 or 1;

R³ is independently selected from H, halogen, or C₁₋₆alkyl-R^{a}.

R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

In a further embodiment, the compounds of the present invention are represented by formula (I) selected from:

N˜3˜-(1-naphthylmethyl)pyridine-2,3-diamine;

6-[2-(1H-indol-6-yl)ethyl]pyridin-2-amine;

6-[2-(2-naphthyl)ethyl]pyridin-2-amine;

6-[2-(3-chloro-1H-indol-6-yl)ethyl]pyridin-2-amine;

N˜3˜-(2-naphthylmethyl)pyridine-2,3-diamine;

N˜3˜-(1,1′-biphenyl-4-ylmethyl)pyridine-2,3-diamine;

N˜3˜-(1,1′-biphenyl-3-ylmethyl)pyridine-2,3-diamine;

6-[2-(3,8-dimethoxy-2-naphthyl)ethyl]pyridin-2-amine;

N—3—-benzyl-6-phenoxypyridine-2,3-diamine;

N—3—-[(3,8-dimethoxy-2-naphthyl)methyl]pyridine-2,3-diamine;

N—3—-(3-pyridin-3-ylbenzyl)pyridine-2,3-diamine;

6-[2-(3-pyridin-3-ylphenyl)ethyl]pyridin-2-amine;

N—3—-(1-biphenylen-2-ylethyl)pyridine-2,3-diamine;

N—3—-[3-(benzyloxy)benzyl]pyridine-2,3-diamine;

N—3—-(1,1′-biphenyl-3-ylmethyl)-6-methylpyridine-2,3-diamine;

N˜3˜-(2-phenylethyl)pyridine-2,3-diamine;

6-methyl-N˜3˜-(3-pyridin-3-ylbenzyl)pyridine-2,3-diamine;

N˜3˜-{[4′-(3-methyl-1H-pyrazol-5-yl)-1,1′-biphenyl-3-yl]methyl}pyridine-2,3-diamine

N˜3˜-[3-(1H-indol-6-yl)benzyl]pyridine-2,3-diamine;

ethyl 3′-{[(2-aminopyridin-3-yl)amino]methyl}-1,1′-biphenyl-3-carboxylate;

(3′-{[(2-aminopyridin-3-yl)amino]methyl}-1,1′-biphenyl-3-yl)acetonitrile;

N˜3˜-(1H-indol-6-ylmethyl)-6-methylpyridine-2,3-diamine;

N˜3˜-{3-[5-(1H-pyrazol-5-yl)thien-2-yl]benzyl}pyridine-2,3-diamine;

N˜3˜-(4-methoxy-2,3-dimethylbenzyl)pyridine-2,3-diamine;

2-(3′-{[(2-aminopyridin-3-yl)amino]methyl}-1,1′-biphenyl-3-yl)ethanol;

(3′-{[(2-aminopyridin-3-yl)amino]methyl}-1,1′-biphenyl-3-yl)methanol;

N˜3˜-{[5-(4-fluorophenyl)pyridin-3-yl]methyl}-6-methylpyridine-2,3-diamine;

N˜3˜-(3-butoxybenzyl)pyridine-2,3-diamine;

N˜3˜-(5-bromo-2-ethoxybenzyl)pyridine-2,3-diamine;

N˜3˜-[3-(cyclopentyloxy)benzyl]pyridine-2,3-diamine;

N˜3˜-[(9-ethyl-9H-carbazol-3-yl)methyl]pyridine-2,3-diamine;

N˜3˜-[3-(1H-indol-6-yl)benzyl]-6-methylpyridine-2,3-diamine;

N˜3˜-(5-bromo-2-methoxybenzyl)-6-methylpyridine-2,3-diamine;

6-methyl-N˜3˜-[(3-phenyl-1,3-dihydro-2,1-benzisoxazol-5-yl)methyl]pyridine-2,3-diamine;

N˜3˜-[5-(1H-indol-6-yl)-2-methoxybenzyl]-6-methylpyridine-2,3-diamine;

N˜3˜-[2-(alkyloxy)-5-bromobenzyl]pyridine-2,3-diamine;

N˜3˜-[2-(benzyloxy)-5-bromobenzyl]pyridine-2,3-diamine;

N˜3˜-[5-bromo-2-(prop-2-ynyloxy)benzyl]pyridine-2,3-diamine;

(3′-{[(2-amino-6-methylpyridin-3-yl)amino]methyl}-4′-methoxy-1,1′-biphenyl-3-yl)acetonitrile;

N˜3˜-[(3′-amino-4-methoxy-1,1′-biphenyl-3-yl)methyl]-6-methylpyridine-2,3-diamine;

N˜(3′-{[(2-amino-6-methylpyridin-3-yl)amino]methyl}-4′-methoxy-1,1′-biphenyl-3-yl)acetamide;

N˜3˜-(5-isoquinolin-4-yl-2-methoxybenzyl)-6-methylpyridine-2,3-diamine;

6-{2-[3-(1H-indol-6-yl)phenyl]ethyl}pyridin-2-amine;

N˜3˜-(3-isoquinolin-4-ylbenzyl)-6-methylpyridine-2,3-diamine;

N˜3˜-[(3′-amino-1,1′-biphenyl-3-yl)methyl]-6-methylpyridine-2,3-diamine;

N˜3˜-[2-(benzyloxy)-5-(1H-indol-6-yl)benzyl]pyridine-2,3-diamine;

5-bromo-N˜3˜-[3-(1H-indol-6-yl)benzyl]pyridine-2,3-diamine;

N˜3˜-[(3′,4-dimethoxy-1,1′-biphenyl-3-yl)methyl]-6-methylpyridine-2,3-diamine;

N˜3˜-[5-bromo-2-(pyridin-2-ylmethoxy)benzyl]pyridine-2,3-diamine;

N˜3˜-[(3′-methoxy-1,1′-biphenyl-3-yl)methyl]-6-methylpyridine-2,3-diamine;

N˜3˜-[5-(1H-indol-6-yl)-2-(pyridin-2-ylmethoxy)benzyl]pyridine-2,3-diamine;

N˜3˜-[2-(benzyloxy)-5-pyridin-3-ylbenzyl]pyridine-2,3-diamine;

5-bromo-N˜3˜-[(3′-methoxy-1,1′-biphenyl-3-yl)methyl]pyridine-2,3-diamine;

3-{[(2-aminopyridin-3-yl)amino]methyl}-3′-methoxy-1,1′-biphenyl-4-ol;

N˜3˜-[3-(2-furyl)benzyl]-6-methylpyridine-2,3-diamine;

N˜3˜-[5-(2-furyl)-2-methoxybenzyl]-6-methylpyridine-2,3-diamine;

N˜3˜-[2-(benzyloxy)-5-tert-butylbenzyl]pyridine-2,3-diamine;

N˜3˜-{[3′-methoxy-4-(pyridin-2-ylmethoxy)-1,1′-biphenyl-3-yl]methyl}pyridine-2,3-diamine;

6-chloro-N˜3˜-[(3′-methoxy-1,1′-biphenyl-3-yl)methyl]pyridine-2,3-diamine;

5-chloro-N˜3˜-[(3′-methoxy-1,1′-biphenyl-3-yl)methyl]pyridine-2,3-diamine;

N˜3˜-{[4-(benzyloxy)-3′-methoxy-1,1′-biphenyl-3-yl]methyl}pyridine-2,3-diamine;

N˜3˜-[5-bromo-2-(pyridin-3-ylmethoxy)benzyl]pyridine-2,3-diamine;

N˜3˜-[5-(1H-indol-6-yl)-2-(pyridin-3-ylmethoxy)benzyl]pyridine-2,3-diamine;

N˜3˜-[5-(1H-indol-6-yl)-2-(pyridin-4-ylmethoxy)benzyl]pyridine-2,3-diamine;

N˜3˜-[3-(6-chloropyridin-3-yl)benzyl]pyridine-2,3-diamine;

N˜3˜-[(3,4′-dimethoxy-1,1′-biphenyl-3-yl)methyl]pyridine-2,3-diamine;

5-chloro-N˜3˜-(3-pyridin-3-ylbenzyl)pyridine-2,3-diamine;

N˜3˜-[3-(5-methoxypyridin-3-yl)benzyl]pyridine-2,3-diamine;

2-[2-(6-aminopyridin-2-yl)ethyl]-4-(1H-indol-6-yl)phenol;

6-{2-[5-(1H-indol-6-yl)-2-(pyridin-2-ylmethoxy)phenyl]ethyl}pyridin-2-amine;

N˜3˜-[3-(2-furyl)benzyl]pyridine-2,3-diamine;

N˜3˜-[2-[2-(dimethylamino)ethoxy]-5-(1H-indol-6-yl)benzyl]pyridine-2,3-diamine;

N˜3-[5-pyridin-3-yl-2-(2-pyrrolidin-1-ylethoxy)benzyl]pyridine-2,3-diamine;

N˜3˜-[5-(1H-indol-6-yl)-2-(2-pyrrolidin-1-ylethoxy)benzyl]pyridine-2,3-diamine;

N˜3˜-(3-pyrimidin-5-ylbenzyl)pyridine-2,3-diamine;

N˜3˜-(5-bromo-2-isobutoxybenzyl)pyridine-2,3-diamine;

6-{2-[3-(5-methoxypyridin-3-yl)phenyl]ethyl}pyridin-2-amine;

6-[2-(3′-methoxy-1,1′-biphenyl-3-yl)ethyl]pyridin-2-amine;

6-{2-[3-(2-furyl)phenyl]ethyl}pyridin-2-amine;

N˜3˜-[3-(2-methyl-1,3-benzothiazol-5-yl)benzyl]pyridine-2,3-diamine;

N˜3˜-[3-(6-methoxypyridin-3-yl)benzyl]pyridine-2,3-diamine;

N˜3˜-[(3′-amino-1,1′-biphenyl-3-yl)methyl]pyridine-2,3-diamine;

6-[2-(3′,4′-dimethoxy-1,1′-biphenyl-3-yl)ethyl]pyridin-2-amine;

N˜3˜-(3-bromobenzyl)-6-(morpholin-4-ylcarbonyl)pyridine-2,3-diamine;

N-(3′-{[(2-aminopyridin-3-yl)amino]methyl}-1,1′-biphenyl-3-yl)propanamide;

N-(3′-{[(2-aminopyridin-3-yl)amino]methyl}-1,1′-biphenyl-3-yl)butanamide;

methyl 3-[(3′-{[(2-aminopyridin-3-yl)amino]methyl}-1,1′-biphenyl-3-yl)amino]-3-oxopropanoate;

2-[(3′-{[(2-aminopyridin-3-yl)amino]methyl}-1,1′-biphenyl-3-yl)amino]-2-oxoethyl acetate;

N-(3′-{[(2-aminopyridin-3-yl)amino]methyl}-1,1′-biphenyl-3-yl)-2-(2-methoxyethoxy)acetamide;

N˜3˜-[3-(5-propoxypyridin-3-yl)benzyl]pyridine-2,3-diamine;

{6-amino-5-[(3-bromobenzyl)amino]pyridin-2-yl}methanol;

{6-amino-5-[(3-pyridin-3-ylbenzyl)amino]pyridin-2-yl}methanol;

2-amino-6-[2-(3′-methoxy-1,1′-biphenyl-3-yl)ethyl]nicotinonitrile.

In a further embodiment, the compounds of the present invention are represented by formula (I) or a pharmaceutically acceptable salt thereof as recited herein for use as a medicament.

In a further embodiment, the compounds of the present invention are represented by formula (I) or a pharmaceutically acceptable salt thereof as recited herein in the manufacture of a medicament for the treatment or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemaim-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia.

In a further embodiment, the compounds of the present invention are represented by a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntingtons Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia comprising administering to a human a therapeutically effective amount of a compound of formula (I) or a pharmaceutically acceptable salt, thereof as defined herein.

In a further embodiment, the compounds of the present invention are represented by a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia comprising administering to a human a therapeutically effective amount of a compound of formula (I) or a pharmaceutically acceptable salt, thereof as defined herein.

In a further embodiment, the compounds of the present invention are represented by a method of treating Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer Disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia by administering to a human a compound of formula (I) or a pharmaceutically acceptable salt, thereof as defined herein and a cognitive and/or memory enhancing agent.

In a further embodiment, the compounds of the present invention are represented by a method of treating Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemaim-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia by administering to a human a compound of formula (I) or a pharmaceutically acceptable salt, thereof as defined herein and a choline esterase inhibitor or anti-inflammatory agent.

In a further embodiment, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In a further embodiment, the compounds of the present invention are represented by a pharmaceutical composition comprising a compound of formula (I) or a pharmaceutically acceptable salt thereof as defined herein together with at least one pharmaceutically acceptable carrier, diluent or excipent.

The definitions set forth in this application are intended to clarify terms used throughout this application. The term “herein” means the entire application.

As used in this application, the term “optionally substituted,” as used herein, means that substitution is optional and therefore it is possible for the designated atom to be unsubstituted. In the event a substitution is desired then such substitution means that any number of hydrogens on the designated atom is replaced with a selection from the indicated group, provided that the normal valency of the designated atom is not exceeded, and that the substitution results in a stable compound. For example when a substituent is keto (i.e., ═O), then 2 hydrogens on the atom are replaced. For example if V is O and n is 1 then m cannot be greater than 1. Examples of such substituents are as follows: halogen, CN, NH₂, OH, SO, SO₂, COOH, OC₁₋₆alkyl, CH₂OH, SO₂H, C₁₋₆alkyl, OC₁₋₆alkyl, C(═O)C₁₋₆alkyl, C(═O)OC₁₋₆alkyl, C(═O)NH₂, C(═O)NHC₁₋₆alkyl, C(═O)N(C₁₋₆alkyl)2, SO₂C₁₋₆alkyl, SO₂NHC₁₋₆alkyl, SO₂N(C₁₋₆alkyl)2, NH(C₁₋₆alkyl), N(C₁₋₆alkyl)2, NHC(═O)C₁₋₆alkyl, NC(═O)(C₁₋₆alkyl)₂, C₅₋₆aryl, OC₅₋₆aryl, C(═O)C₅₋₆aryl, C(═O)OC₅₋₆aryl, C(═O)NHC₅₋₆aryl, C(═O)N(C₅₋₆aryl)₂, SO₂C₅₋₆aryl, SO₂NHC₅₋₆aryl, SO_{N(C}₅₋₆aryl)₂NH(C₅₋₆aryl), N(C₅₋₆aryl)2NC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(═O)C₅₋₆heterocyclyl, NC(═O)(C₅₋₆heterocyclyl)₂;

A variety of compounds in the present invention may exist in particular geometric or stereoisomeric forms. The present invention takes into account all such compounds, including cis- and trans isomers, R— and S— enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl” or “alkylene” used alone or as a suffix or prefix, is intended to include both branched and straight-chain saturated aliphatic hydrocarbon groups having from 1 to 12 carbon atoms or if a specified number of carbon atoms is provided then that specific number would be intended. For example “C₁₋₆ alkyl” denotes alkyl having 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. As used herein, “C₁₋₃alkyl”, whether a terminal substituent or an alkylene group linking two substituents, is understood to specifically include both branched and straight-chain methyl, ethyl, and propyl.

As used herein, “alkenyl” refers to hydrocarbyl groups having at least one carbon-carbon double bond in the ring, and having from 2 to 6 carbons atoms. For example “C₂₋₆alkenyl” denotes alkenyl having 2, 3, 4, 5 or 6 carbon atoms. Examples of alkenyl include, but are not limited to, ethenyl (vinyl), 1-propenyl, 2-propenyl (alkyl), isopropenyl, butenyl, buta-1,4-dienyl, pentenyl, and hexenyl. Particular examples of C₂₋₆alkenyl groups are C₂₋₄alkenyl groups.

As used herein, “alkynyl” refers to hydrocarbyl groups having at least one carbon-carbon triple bond in the ring, and having from 2 to 6 carbons atoms. For example “C₂₋₆alkynyl” denotes alkynyl having 2, 3, 4, 5 or 6 carbon atoms. Examples of alkynyl groups include, but are not limited to, ethynyl and 2-propynyl (propargyl) groups. Particular examples of C₂₋₆ alkynyl groups are C₂₋₄alkynyl groups.

As used herein, “aromatic” refers to hydrocarbyl groups having one or more polyunsaturated carbon rings having aromatic character, (e.g., 4n+2 delocalized electrons) and comprising up to about 14 carbon atoms.

As used herein, the term “aryl” refers to a ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, “cycloalkyl” refers to non-aromatic cyclic hydrocarbons including cyclized alkyl, alkenyl, and alkynyl groups, having the specified number of carbon atoms. Cycloalkyl groups can include mono- or polycyclic (e.g., having 2, 3 or 4 fused or bridged rings) groups. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbomyl, norpinyl, norcarnyl, adamantyl, and the like. Also included in the definition of cycloalkyl are moieties that have one or more aromatic rings fused (i.e., having a bond in common with) to the cycloalkyl ring, for example, benzo derivatives of cyclopentane (i.e., indanyl), cyclopentene, cyclohexane, and the like. The term “cycloalkyl” further includes saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in their ring structure. For example, “C₃₋₆ cycloalkyl” denotes such groups as cyclopropyl, cyclobutyl, cyclopentyl, or cyclohexyl.

As used herein, “cycloalkenyl” refers to ring-containing hydrocarbyl groups having at least one carbon-carbon double bond in the ring, and having from 3 to 12 carbons atoms.

As used herein, “cycloalkynyl” refers to ring-containing hydrocarbyl groups having at least one carbon-carbon triple bond in the ring, and having from 7 to 12 carbons atoms.

As used herein, “halo” or “halogen” refers to fluoro, chloro, bromo, and iodo. “Counterion” is used to represent a small, negatively charged species such as chloride, bromide, hydroxide, acetate, sulfate, tosylate, benezensulfonate, and the like.

As used herein, the term “heterocyclyl” or “heterocyclic” or “heterocycle” refers to a ring-containing monovalent and divalent structures having one or more heteroatoms, independently selected from N, O and S, as part of the ring structure and comprising from 3 to 20 atoms in the rings, more preferably 3- to 7-membered rings. Heterocyclic groups may be saturated or partially saturated or unsaturated, containing one or more double bonds, and heterocyclic groups may contain more than one ring as in the case of polycyclic systems. The heterocyclic rings described herein may be substituted on carbon or on a heteroatom atom if the resulting compound is stable. If specifically noted, nitrogen in the heterocyclyl may optionally be quatemized. It is understood that when the total number of S and O atoms in the heterocyclyl exceeds 1, then these heteroatoms are not adjacent to one another. The number of ring-forming atoms in heterocyclyl is given in ranges herein. For example, C₅₋₁₀ heterocyclyl refers to a ring structure comprising from 5 to 10 ring forming atoms wherein at least one of the ring forming atoms is N, O or S. Examples of heterocyclyls include, but are not limited to, 1H-indazole, 2-pyrrolidonyl, 2H, 6H-1, 5,2-dithiazinyl, 2H-pyrrolyl, 3H-indolyl, 4-piperidonyl, 4H-carbazole, 4H-quinolizinyl, 6H-1, 2,5-thiadiazinyl, acridinyl, azabicyclo, azetidine, azepane, aziridine, azocinyl, benzimidazolyl, benzodioxol, benzofuranyl, benzothiofuranyl, benzothiophenyl, benzoxazolyl, benzthiazolyl, benzotriazolyl, benzotetrazolyl, benzisoxazolyl, benzisothiazolyl, benzimidazalonyl, carbazolyl, 4H-carbazolyl, b-carbolinyl, chromanyl, chromenyl, cinnolinyl, diazepane, decahydroquinolinyl, 2H,6H-1,5,2-dithiazinyl, dioxolane, furyl, 2,3-dihydrofuran, 2,5-dihydrofuran, dihydrofuro[2,3-b]tetrahydrofuran, furanyl, furazanyl, homopiperidinyl, imidazolidine, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxirane, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, purinyl, pyranyl, pyrrolidinyl, pyrroline, pyrrolidine, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolidinyl dione, pyrrolinyl, pyrrolyl, pyridine, quinazolinyl, quinolinyl, 4H-quinolizinyl, quinoxalinyl, quinuclidinyl, carbolinyl, tetrahydrofuranyl, tetramethylpiperidinyl, tetrahydroquinoline, tetrahydroisoquinolinyl, thiophane, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiopheneyl, thiirane, triazinyl, 1,2,3-triazolyl, 1,2,4-triazolyl, 1,2,5-triazolyl, 1,3,4-triazolyl, xanthenyl.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, alkyloxy and propargyloxy. Similarly, “alkylthio” or “thioalkoxy” represent an alkyl group as defined above with the indicated number of carbon atoms attached through a sulphur bridge.

As used herein, the term “carbonyl” is art recognized and includes such moieties as can be represented by the general formula:

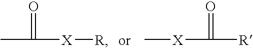

wherein X is a bond or represents an oxygen or sulfur, and R represents a hydrogen, an alkyl, an alkenyl, —(CH₂)ₘ—R″ or a pharmaceutically acceptable salt, R¹ represents a hydrogen, an alkyl, an alkenyl or —(CH₂)ₘ—R″, where m is an integer less than or equal to ten, and R″ is alkyl, cycloalkyl, alkenyl, aryl, or heteroaryl. Where X is an oxygen and R and R′ is not hydrogen, the formula represents an “ester”. Where X is an oxygen, and R is as defined above, the moiety is referred to herein as a carboxyl group, and particularly when R′ is a hydrogen, the formula represents a “carboxylic acid.” Where X is oxygen, and R′ is a hydrogen, the formula represents a “formate.” In general, where the oxygen atom of the above formula is replaced by sulfur, the formula represents a “thiolcarbonyl” group. Where X is a sulfur and R and R′ is not hydrogen, the formula represents a “thiolester.” Where X is sulfur and R is hydrogen, the formula represents a “thiolcarboxylic acid.” Where X is sulfur and R′ is hydrogen, the formula represents a “thiolformate.” On the other hand, where X is a bond, and R is not a hydrogen, the above formula represents a “ketone” group. Where X is a bond, and R is hydrogen, the above formula is represents an “aldehyde” group.

As used herein, the term “sulfonyl” refers to a moiety that can be represented by the general formula:

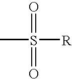

wherein R is represented by but not limited to hydrogen, alkyl, cycloalkyl, alkenyl, aryl, heteroaryl, aralkyl, or heteroaralkyl.

As used herein, some substituents are discribed in a combination of two or more groups. For example, the expression of “C(═O)C₃₋₉cycloalkylR^{d}” is meant to refer to a structure:

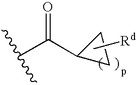

wherein p is 1, 2, 3, 4, 5, 6 or 7 (a C₃₋₉cycloalkyl); the C₃₋₉cycloalkyl is substituted by R^{d}; and the point of attachment of the “C(═O)C₃₋₉cycloalkylR^{d}” is through the carbon atom of the carbonyl group, which is on the left of the expression.

As used herein some substitutents can occur at multiple times. For example, the expression of “C₁₋₆alkylNHC₅₋₉heterocyclyl(R^{d})ₜ” is meant to ferer to R^{d} can can occur on the heterocyclyl moiety portion t times and R^{d} can be a different substituent in its definition at each occurrence.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: N.Y., 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, phosphoric, and the like; and the salts prepared from organic acids such as lactic, maleic, citric, benzoic, methanesulfonic, and the like. As used herein ‘pharmaceutically acceptable salts’ refers also to any solvates e.g. hydrates of the compounds of the present invention including compounds of formula I and salts thereof.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used.

As used herein, “in vivo hydrolysable precursors” means an in vivo hydroysable (or cleavable) ester of a compound of the formula (I) that contains a carboxy or a hydroxy group. For example amino acid esters, C₁₋₆ alkoxymethyl esters like methoxymethyl; C₁₋₆alkanoyloxymethyl esters like pivaloyloxymethyl; C₃₋₈cycloalkoxycarbonyloxy C₁₋₆alkyl esters like 1-cyclohexylcarbonyloxyethyl, acetoxymethoxy, or phosphoramidic cyclic esters.

As used herein, “tautomer” means other structural isomers that exist in equilibrium resulting from the migration of a hydrogen atom. For example, keto-enol tautomerism where the resulting compound has the porperties of both a ketone and an unsturated alchol.

As used herein “stable compound” and “stable structure” are meant to indicate a compound that is sufficiently robust to survive isolation to a useful degree of purity from a reaction mixture, and formulation into an efficacious therapeutic agent.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I , ¹³¹I, ³⁵S or will generally be most useful. For radio-imaging applications ¹¹C , ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

The anti-dementia treatment defined herein may be applied as a sole therapy or may involve, in addition to the compound of the invention, conventional chemotherapy. Such chemotherapy may include one or more of the following categories of agents: acetyl cholinesterase inhibitors, anti-inflammatory agents, cognitive and/or memory enhancing agents or atypical antipsychotic agents.

Such conjoint or concurrent treatment may be achieved by way of the simultaneous, sequential or separate dosing of the individual components of the treatment. Such combination products employ the compounds of this invention.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in is patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, 5 pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. For example, such conventional non-toxic salts include those derived from inorganic acids such as hydrochloric, phosphoric, and the like; and the salts prepared from organic acids such as lactic, maleic, citric, benzoic, methanesulfonic, trifluoroacetate and the like.

In one embodiment a compound of the formula (I) or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finally divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension.

If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), esters such as in vivo hydrolysable esters, free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Compounds having acidic groups, such as carboxylate, phosphates or sulfates, can form salts with alkaline or alkaline earth metals such as Na, K, Mg and Ca, and with organic amines such as triethylamine and Tris (2-hydroxyethyl)amine. Salts can be formed between compounds with basic groups, e.g. amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Acid addition salts may be formed with a wide variety of acids, both inorganic and organic. Examples of acid addition salts include salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

If the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR⁴⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages.

More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Esters can be formed between hydroxyl or carboxylic acid groups present in the compound and an appropriate carboxylic acid or alcohol reaction partner, using techniques well known in the art. Examples of esters are compounds containing the group —C(═O)OR, wherein R is an ester substituent, for example, a C₁₋₇alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇alkyl group, a C₃₋₂₀heterocyclyl group, or a C₅₋₂₀ aryl group, preferably a C₁₋₇alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Derivatives which are prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it. Some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is: C₁₋₇alkyl (e.g., -Me, -Et, -nPr, -iPr, -nBu, -sBu, -iBu, -tBu); C₁₋₇aminoalkyl (e.g., aminoethyl; 2-(N,N-diethylamino)ethyl; 2-(4-morpholino)ethyl); and acyloxy-C₁₋₇alkyl (e.g., acyloxymethyl; acyloxyethyl; pivaloyloxymethyl; acetoxymethyl; 1-acetoxyethyl; 1-(1-methoxy-1-methyl)ethyl-carbonyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetahydropyranyloxy)carbonyloxyethyl;(4-tetrahydropyranyl)carbonyloxymethyl; and 1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention. Ultimately, the quantity of compound administered will be commensurate with the nature of the disease or physiological condition being treated and will be at the discretion of the physician.

Compounds of formula (I) have been shown to inhibit beta secretase activity in vitro. Inhibitors of beta secretase have been shown to be useful in blocking formation or aggregation of Aβ peptide and therefore have beneficial effects in treatment of Alzheimer's Disease and other neurodegenerative diseases associated with elevated levels and/or Is deposition of Aβ peptide. Therefore it is believed that the compounds of formula (I) may be used for the treatment of Alzheimer disease and disease associated with dementia Hence compounds of formula (I) and their salts are expected to be active against age-related diseases such as Alzheimer, as well as other Aβ related pathologies such as Downs syndrome and b-amyloid angiopathy. It is expected that the compounds of formula (I) would most likely be used in combination with a broad range of cognition deficit enhancement agents but could also be used as a single agent.

Generally, the compounds of formula (I) have been identified in at least one of the assays described below as having an IC₅₀ value of 300 micromolar or less. The preferred compounds of formula (I) have been identified in at least one of the assays described below as having an IC₅₀ value of 25 micromolar or less.

IGEN Assay

Enzyme is diluted 1:30 in 40 mM MES pH 5.0. Stock substrate is diluted to 12 uM in 40 mM MES pH 5.0. PALMEB solution is added to the substrate solution (1:100 dilution). DMSO stock solutions of compounds or DMSO alone are diluted to the desired concentration in 40 mM MES pH 5.0. The assay is done in a 96 well PCR plate from Nunc. Compound in DMSO (3 uL) is added to the plate then enzyme is added (27 uL) and pre-incubated with compound for 5 minutes. Then the reaction is started with substrate (30 uL). The final dilution of enzyme is 1:60; the final concentration of substrate is 6 uM (Km is 150 uM). After a 20 minute reaction at room temperature, the reaction is stopped by removing 10 ul of the reaction mix and diluting it 1:25 in 0.20 M Tris pH 8.0. The compounds are added to the plate by hand then all the rest of the liquid handling is done on the CyBi-well instrument.

All antibodies and the streptavidin coated beads are diluted into PBS containing 0.5% BSA and 0.5% Tween20. The product is quantified by adding 50 uL of a 1:5000 dilution of the neoepitope antibody to 50 uL of the 1:25 dilution of the reaction mix. Then, 100 uL of PBS (0.5% BSA, 0.5% Tween20) containing 0.2 mg/ml IGEN beads and a 1:5000 dilution of ruthinylated goat anti-rabbit (Ru-Gar) antibody is added. The final dilution of neoepitope antibody is 1:20,000, the final dilution of Ru-GAR is 1:10,000 and the final concentration of beads is 0.1 mg/ml. The mixture is read on the IGEN instrument with the CindyAB40 program after a 2-hour incubation at room temperature. Addition of DMSO alone is used to define the 100% activity. 20 uM control inhibitor is used to define 0% of control activity and 100 nM inhibitor defines 50% control of control activity in single-poke assays. Control 20 inhibitor is also used in dose response assays with an IC50 of 100 nM.

Fluorescent Assay

Enzyme is diluted 1:30 in 40 mM MES pH 5.0. Stock substrate is diluted to 30 uM in 40 mM MES pH 5.0. PALMEB solution is added to the substrate solution (1:100 dilution). Enzyme and substrate stock solutions are kept on ice until the placed in the stock plates. The Platemate-plus instrument is used to do all liquid handling. Enzyme (9 uL) is added to the plate then 1 uL of compound in DMSO is added and pre-incubated for 5 minutes. When a dose response curve is being tested for a compound, the dilutions are done in neat DMSO and the DMSO stocks are added as described above. Substrate (10 uL) is added 30 and the reaction proceeds in the dark for 1 hour at room temperature. The assay is done in a Coming 384 well round bottom, low volume, non-binding surface (Coming #3676). The final dilution of enzyme is 1:60; the final concentration of substrate is 15 uM (Km of 25 uM). The fluorescence of the product is measured on a Victor II plate reader with an excitation wavelength of 360 nm and an emission wavelength of 485 nm using the protocol labeled Edans peptide. The DMSO control defines the 100% activity level and 0% activity is defined by using 50 uM of the control inhibitor, which completely blocks enzyme function. The control inhibitor is also used in dose response assays and has an IC50 of 95 nM.

Beta-Secretase Whole Cell Assay

Generation of HEK-Fc33-1.

The cDNA encoding fall length BACE was fused in frame with a three amino acid linker (Ala-Val-Thr) to the Fc portion of the human IgG1 starting at amino acid 104. The BACE-Fc construct was then cloned into an expression vector, including an IRES control region, neoK resistance gene and a green fluorescent protein (GFP) gene, for protein expression in mammalian cells. The expression vector was stably transfected into HEK-293 cells using a calcium phosphate method. Colonies were selected with 250 μg/mL of G-418. Limited dilution cloning was performed to generate homogeneous cell lines. Clones were characterized by levels of APP expression and Aβ secreted in the conditioned media using an ELISA assay developed in-house. Aβ secretion of BACE/Fc clone Fc33-1 was moderate.

Cell Culture

HEK293 cells stably expressing human BACE (HEK-Fc33) were grown at 37° C. in DMEM containing 10% heat-inhibited FBS, 0.5 mg/mL antibiotic-antimycotic solution, and 0.05 mg/mL of the selection antibiotic G-418.

Aβ40 Release Assay.

Cells were harvested when between 80 to 90% confluent. 100 μL of cells at a cell density of 1.5 million/mL were added to a white 96-well cell culture plate with clear flat bottom (Costar 3610), or a clear, flat bottom 96-well cell culture plate (Costar 3595), containing 100 μL of inhibitor in cell culture medium with DMSO at a final concentration of 1%.

After the plate was incubated at 37° C. for 24 h, 100 μL cell medium was transferred to a round bottom 96-well plate (Costar 3365) to quantify Aβ40 levels. The cell culture plates were saved for ATP assay as described in ATP assay below. To each well of the round bottom plate, 50 μL of detection solution containing 0.2 μg/mL of the RαAβ40 antibody and 0.25 μg/mL of a biotinylated 4G8 antibody (prepared in DPBS with 0.5% BSA and 0.5% Tween-20) was added and incubated at 4° C. for at least 7 h. Then a 50 μL solution (prepared in the same buffer as above) containing 0.062 μg/mL of a ruthenylated goat anti-rabbit antibody and 0.125 mg/mL of streptavidin coated Dynabeads was added per well. The plate was shaken at 22° C. on a plate shaker for 1 h, and then the plates were then measured for ECL counts in an IGEN M8 Analyzer. Aβ standard curves were obtained with 2-fold serial dilution of an Aβ stock solution of known concentration in the same cell culture medium used in cell-based assays.

ATP Assay.

As indicated above, after transferring 100 μL medium from cell culture plates for Aβ40 detection, the plates, which still contained cells, were saved for cytotoxicity assays by using the assay kit (ViaLigh™ Plus) from Cambrex BioScience that measures total cellular ATP. Briefly, to each well of the plates, 50 μL cell lysis reagent was added. The plates were incubated at room temperature for 10 min. Two min following addition of 100 μL reconstituted ViaLight™ Plus reagent for ATP measurement, the luminescence of each well was measured in an LJL plate reader or Wallac Topcount.

BACE Biacore Protocal

Sensor Chip Preparation:

BACE was assayed on a Biacore3000 instrument by attaching either a peptidic transition state isostere (TSI) or a scrambled version of the peptidic TSI to the surface of a Biacore CM5 sensor chip. The surface of a CM5 sensor chip has 4 distinct channels that can be used to couple the peptides. The scrambled peptide KFES-statine-ETIAEVENV was coupled to channel 1 and the TSI inhibitor KTEEISEVN-statine-VAEF was couple to channel 2 of the same chip. The two peptides were dissolved at 0.2 mg/ml in 20 mM Na Acetate pH 4.5, and then the solutions were centrifuged at 14K rpm to remove any particulates. Carboxyl groups on the dextran layer were activated by injecting a one to one mixture of 0.5M N-ethyl-N′ (3-dimethylaminopropyl)-carbodiimide (EDC) and 0.5M N-hydroxysuccinimide (NHS) at 5 uL/minute for 7 minutes. Then the stock solution of the control peptide was injected in channel 1 for 7 minutes at 5 uL/min., and then the remaining activated carboxyl groups were blocked by injecting 1M ethanolamine for 7 minutes at 5 uL/minute.

Assay Protocol:

The BACE Biacore assay was done by diluting BACE to 0.5 μM in Na Acetate buffer at pH 4.5 (running buffer minus DMSO). The diluted BACE was mixed with DMSO or compound diluted in DMSO at a final concentration of 5% DMSO. The BACE/inhibitor mixture was incubated for 1 hour at 4° C. then injected over channel 1 and 2 of the CM5 Biacore chip at a rate of 20 μL/minute. As BACE bound to the chip the signal was measured in response units (RU). BACE binding to the TSI inhibitor on channel 2 gave a certain signal. The presence of a BACE inhibitor reduced the signal by binding to BACE and inhibiting the interaction with the peptidic TSI on the chip. Any binding to channel 1 was non-specific and was subtracted from the channel 2 responses. The DMSO control was defined as 100% and the effect of the compound was reported as percent inhibition of the DMSO control.

BACE Activity Assay

Method 1

Activity of the BACE enzyme was measured using the peptide R-E(EDANS)-E-V—N-L-*D-A-E-F—K(DABCYL)-R—OH from Bachem as substrate 1. Compounds were incubated with BACE and 10 uM peptide substrate 1 in 50 mM sodium acetate, pH 5, 10% DMSO in 96-well black, flat bottomed Cliniplates in a final assay volume of 100 ul. The reaction rate was monitored at room temperature on a Fluoroskan Ascent platereader with excitation and emission wavelengths of 355 nm and 530 nm respectively. Initial reaction rates were measured and IC50s were calculated from replicate curves using GraphPad Prizm software.

Method 2

Compounds that fluoresced under the conditions described above were assayed in an alternative assay. Compounds were incubated with BACE and 0.25 uM peptide substrate 2 (PanVera, kit P2985) in 50 mM sodium acetate, pH 4.5 (provided with kit), 5% DMSO in 96-well black, flat bottomed ½ area Costar plates in a final assay volume of 50 ul. The reaction rate was monitored at room temperature on a SpectraMax Gemini XS platereader (Molecular Devices) with excitation and emission wavelengths of 545 nm and 595 nm respectively. Initial reaction rates were measured and used to calculate IC50s as described above.

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those is skilled in the art. Such methods include, but are not limited to, those described below. All references cited herein are hereby incorporated in their entirety by reference.

The novel compounds of this invention may be prepared using the reactions and techniques described herein. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents, which are not compatible with the reaction conditions, will be readily apparent to one skilled in the art and alternate methods must then be used.

The starting materials for the examples contained herein are either commercially available or are readily prepared by standard methods from known materials. For example the following reactions are illustrations but not limitations of the preparation of some of the starting materials and examples used herein.

General procedures for making the compounds of the invention are outlined below. Compounds of Formula I where Z is N and s is 1, Y is C₁₋₆alkyl e.g. C₁-alkyl and r is 1 and q is 0 can be synthesized as outlined in Scheme 1 below.

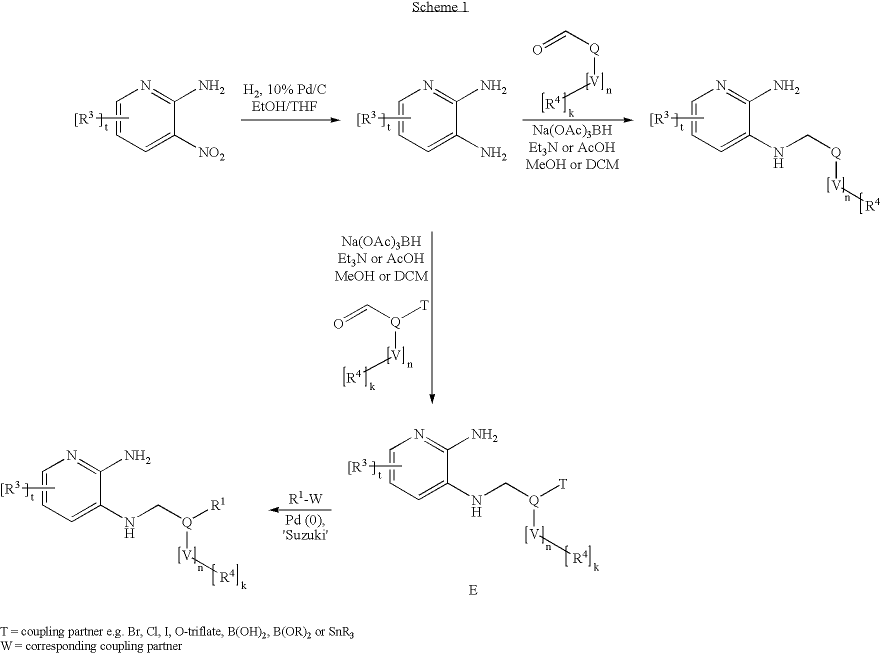

Scheme 1 illustrates the general approach to preparation of examples containing a 2,3-diaminopyridine moiety. A similar process could equally be utilized to synthesise compounds with the alternative substitution pattern e.g. 2,6-disubstituted pyridines. The 2,3-diaminopyridine scaffolds were either commercially available, or made from the commercially available nitro-compound by hydrogenation. Reductive amination, or less frequently alkylation, allowed functionalisation of the 3-amino group. In some cases, further substitution of this group was carried out using palladium mediated Suzuki reaction.

In one embodiment of the present invention, there is provided a compound according to Formula (E):

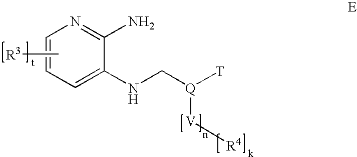

wherein T is a coupling partner, R³, R⁴, Q, V, t, n and k are as defined above, for Formula (I).

Said coupling partner T is for example Br, Cl, I, O-triflate, B(OH)₂, B(OR)₂ or SnR₃.

The compounds of Formula (E) are useful as chemical intermediates in the preparation of compounds of formula I, where Z is N and s is 1, Y is C₁₋₆alkyl e.g. C₁-alkyl and r is 1 and q is 0.

Consequently, in a further embodiment of the present invention there is provided a use of a compound of Formula (E) as a chemical intermediate in the preparation of a compound of formula I, wherein Z is N, s is 1, Y is C₁₋₆alkyl, r is 1 and q is 0.

In some of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999). In particular the tert-butoxycarbonyl (BOC) protecting group may be used.

Examples of functional group interconversions, such as reduction of aromatic nitro compounds, reductive amination and alkylation, and reagents and conditions for carrying out such conversions can be found in, for example, Advanced Organic Chemistiy, by Jerry March, 4^{th} edition, 119, Wiley Interscience, New York, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8).

In the preparative procedures outlined above, the coupling of the aryl or heteroaryl R¹ group is accomplished by reacting a halo-aryl or heteroaryl compound with a boronate ester or boronic acid in the presence of a palladium catalyst and base. Many boronates suitable for use in preparing compounds of the invention are commercially available, for example from Boron Molecular Limited of Noble Park, Australia, or from Combi-Blocks Inc, of San Diego, USA. Where the boronates are not commercially available, they can be prepared by methods known in the art, for example as described in the review article by N. Miyaura and A. Suzuki, Chem. Rev. 1995, 95, 2457. Thus, boronates can be prepared by reacting the corresponding bromo-compound with an alkyl lithium such as butyl lithium and then reacting with a borate ester. The resulting boronate ester derivative can, if desired, be hydrolysed to give the corresponding boronic acid. In the coupling reaction it is understood that T or W can be one coupling partner e.g. the boronic acid or tin compound (where R is alkyl) and the corresponding coupling partner i.e. the halogen or triflate.

Olefins can also be added in this way and can also be introduced by use of the Heck reaction.

Compounds with alternative linkers can be synthesised using methodologies outlined in the literature for example where Y is branched or straight chain C₁₋₆ alkyl group halo-alkylation methodologies can be used.

Compounds of Formula I where Z is C₁-alkyl and s is 1, and Y is C₁₋₆alkyl e.g. C₁-alkyl and r is 1, and q is 0 can be synthesized as outlined in Scheme 2 below.

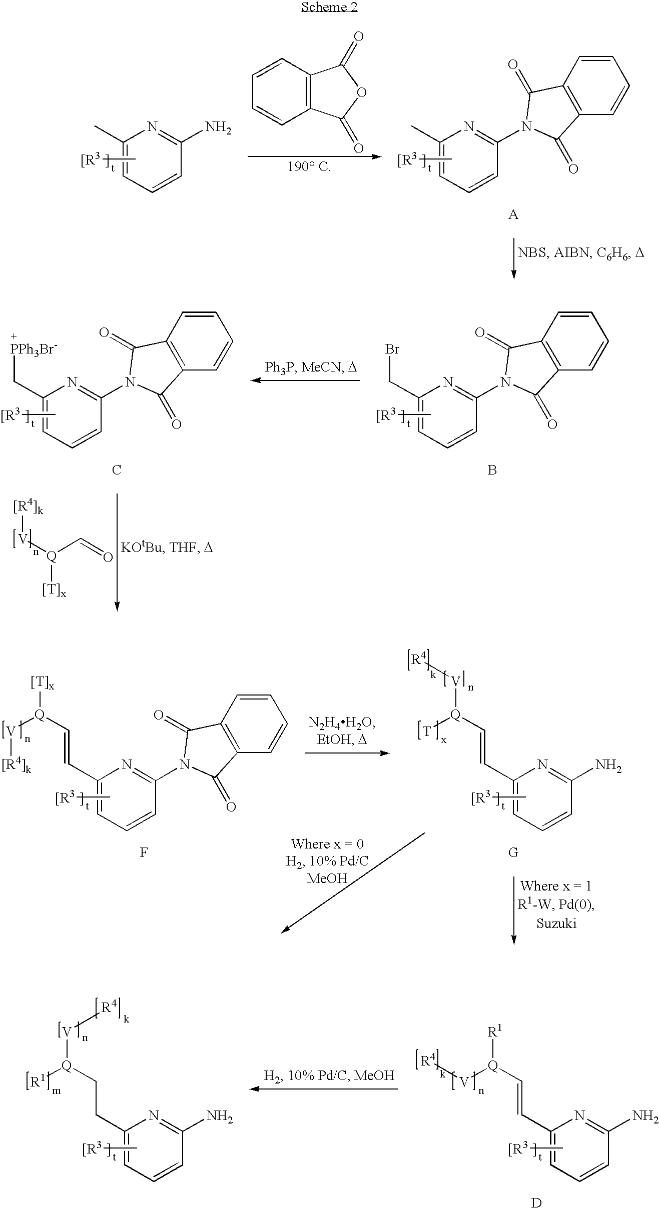

Scheme 2 illustrates the general approach to examples containing 6-substitution on the 2-aminopyridine moiety. A similar process could equally be utilized to synthesise compounds with the alternative substitution pattern e.g. 2,3-disubstituted pyridines. Compounds A and B were prepared using standard methodologies as described in the literature and the phosphonium salt C was then the common intermediate for further synthesis (Advanced Organic Chemistry, by Jerry March, 4^{th} edition, 119, Wiley Interscience, New York, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8)). Wittig reactions were used to install the 6-substituent, followed by reduction of the olefin. The aryl substituent could also be modified by using palladium mediated Suzuki reactions to form bi-aryl examples.

In one embodiment of the present invention, there is provided a compound according to Formula (F):

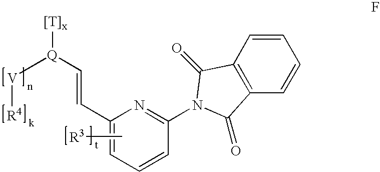

wherein T is a coupling partner, x is 0 or 1, R³, R⁴, Q, V, t, n and k are as defined above, for Formula (I).

Said coupling partner T is for example Br, Cl, I, O-triflate, B(OH)₂, B(OR)₂ or SnR₃.

The compounds of Formula (F) are useful as chemical intermediates in the preparation of compounds of formula I, where Z is C₁-alkyl and s is 1, and Y is C₁₋₆alkyl e.g. C₁-alkyl and r is 1, and q is 0.

Consequently, in a further embodiment of the present invention there is provided a use of a compound of Formula (F) as a chemical intermediate in the preparation of a compound of formula I, wherein Z is C₁-alkyl, s is 1,Y is C₁₋₆alkyl, r is 1 and q is 0.

In a further embodiment of the present invention, there is provided a compound according to Formula (G):

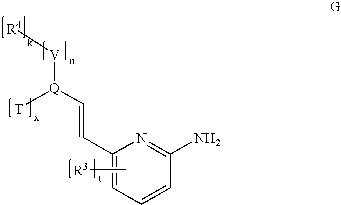

wherein T is a coupling partner, x is 0 or 1, R³, R⁴, Q, V, t, n and k are as defined above, for Formula (I).

Said coupling partner T is for example Br, Cl, I, O-triflate, B(OH)₂, B(OR)₂ or SnR₃.

The compounds of Formula (G) are useful as chemical intermediates in the preparation of compounds of formula I, where Z is C₁-alkyl and s is 1, and Y is C₁₋₆alkyl e.g. C₁-alkyl and r is 1, and q is 0.

Consequently, in a further embodiment of the present invention there is provided a use of a compound of Formula (G) as a chemical intermediate in the preparation of a compound of formula I, wherein Z is C₁-alkyl, s is 1, Y is C₁₋₆alkyl, r is 1 and q is 0.

In a further embodiment of the present invention, there is provided a compound according to Formula (D):

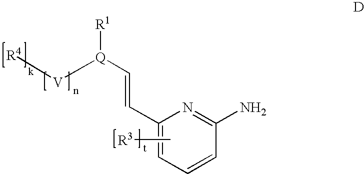

wherein R¹, R³, R⁴, Q, V, t, n and k are as defined above, for Formula (I).

The compounds of Formula (D) are useful as chemical intermediates in the preparation of compounds of formula I, where Z is C₁-alkyl and s is 1, and Y is C₁₋₆alkyl e.g. C₁-alkyl and r is 1, and q is 0.

Consequently, in a further embodiment of the present invention there is provided a use of a compound of Formula (D) as a chemical intermediate in the preparation of a compound of formula I, wherein Z is C₁-alkyl, s is 1,Y is C₁₋₆alkyl, r is 1 and q is 0.

Examples of functional group interconversions, such as bromination, Wittig reactions of olefins, preparation and use of Grignard reactions and catalytic reduction of olefins, and reagents and conditions for carrying out such conversions can be found in, for example, Advanced Organic Chemistry, by Jerry March, 4^{th} edition, 119, Wiley Interscience, New York, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8).

In some of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999). In particular the phthamilide protecting group may be used.

In the preparative procedures outlined above, the coupling of the aryl or heteroaryl R¹ group is accomplished by reacting a halo-aryl or heteroaryl compound with a boronate ester or boronic acid in the presence of a palladium catalyst and base. Many boronates suitable for use in preparing compounds of the invention are commercially available, for example from Boron Molecular Limited of Noble Park, Australia, or from Combi-Blocks Inc, of San Diego, USA. Where the boronates are not commercially available, they can be prepared by methods known in the art, for example as described in the review article by N. Miyaura and A. Suzuki, Chem. Rev. 1995, 95, 2457. Thus, boronates can be prepared by reacting the corresponding bromo-compound with an alkyl lithium such as butyl lithium and then reacting with a borate ester. The resulting boronate ester derivative can, if desired, be hydrolysed to give the corresponding boronic acid. One embodiment of the present invention are novel intermediates.

The invention will now be illustrated by the following non-limiting examples, in which, unless stated otherwise:
I. temperatures are given in degrees Celsius (° C.); unless otherwise stated, operations were carried out at room or ambient temperature, that is, at a temperature in the range of 18-25° C.;
II. organic solutions were dried over anhydrous magnesium sulfate; evaporation of solvent was carried out using a rotary evaporator under reduced pressure (600-4000 Pascals; 4.5-30 mm Hg) with a bath temperature of up to 60° C.;
III. chromatography means flash chromatography on silica gel; thin layer chromatography (TLC) was carried out on silica gel plates;
IV. in general, the course of reactions was followed by TLC or HPLC and reaction times are given for illustration only;
V. melting points are uncorrected and (dec) indicates decomposition;
VI. final products had satisfactory proton nuclear magnetic resonance (NMR) spectra;
VII. when given, NMR data is in the form of delta values for major diagnostic protons, given in parts per million (ppm) relative to tetramethylsilane (TMS) as an internal standard, determined at 400 MHz using deuterated chloroform (CDCl₃), dimethylsulphoxide (d₆-DMSO) or deuterated methanol (d₆-MeOD) as solvent; conventional abbreviations for signal shape are used; for AB spectra the directly observed shifts are reported; coupling constants (J) are given in Hz; Ar designates an aromatic proton when such an assignment is made;
VIII. reduced pressures are given as absolute pressures in pascals (Pa); elevated pressures are given as gauge pressures in bars;
IX. non-aqueous reactions were run under a nitrogen atmosphere
X. solvent ratios are given in volume:volume (v/v) terms; and
XI. Mass spectra (MS) were run using an automated system with atmospheric pressure chemical (APCI) or electrospray (+ES) ionization. Generally, only spectra where parent masses are observed are reported. The lowest mass major ion is reported for molecules where isotope splitting results in multiple mass spectral peaks (for example when chlorine is present).
XII. Commercial reagents were used without further purification.
XIII. Room temperature refers to 20-25° C.
XIV. Analytical LC-MS System: In the examples, the compounds prepared were characterised by liquid chromatography and mass spectroscopy using the systems and operating conditions set out below. Where chlorine is present, if a single mass is present the mass quoted for the compound is for ³⁵Cl. Several systems were used, as described below, and these were equipped with were set up to run under closely similar operating conditions. The operating conditions used are also described below.

Hardware: HPLC System: Waters 2795, Mass Spec Detector: Micromass Platform LC, PDA Detector: Waters 2996 PDA.

Acidic Analytical conditions: Eluent A: H₂O (0.1% Formic Acid), Eluent,B: CH₃CN (0.1% Formic Acid), Gradient: 5-95% eluent B over 3.5 minutes, Flow: 0.8 ml/min, Column: Phenomenex Synergi 4μ MAX-RP 80A, 2.0×50 mm

Basic Analytical conditions: Eluent A: H₂O (10 mM NH₄HCO₃ buffer adjusted to pH=9.5 or pH=9.2 with NH₄OH), Eluent B: CH₃CN, Gradient: 05-95% eluent B over 3.5 minutes,

Flow: 0.8 ml/min, Column:Thermo Hypersil-Keystone BetaBasic-18 5 μm 2.1×50 mm or Phenomenex Luna C18(2) 5 μm 2.0×50 mm

MS conditions: Capillary voltage: 3.6 kV, Cone voltage: 30 V, Source Temperature: 120° C., Scan Range: 165-700 or 125-800 amu, Ionisation Mode: ElectroSpray Positive or ElectroSpray Negative or ElectroSpray Positive & Negative
XV. Mass Directed Purification LC-MS System: Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

One such system for purifying compounds via preparative LC-MS is described below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

Hardware: Waters Fractionlynx system, 2767 Dual Autosampler/Fraction Collector, 2525 preparative pump, CFO (column fluidic organiser) for column selection, RMA (Waters reagent manager) as make up pump, Waters ZQ Mass Spectrometer, Waters 2996 Photo Diode Array detector.

Software: Masslynx 4.0

Columns:

1. Low pH chromatography: Phenomenex Synergy MAX-RP, 10μ 150×15 mm (alternatively used same column type with 100×21.2 mm dimensions).

2. High pH chromatography: Phenomenex Luna C18 (2), 10μ, 100×21.2 mm (alternatively used Thermo Hypersil Keystone BetaBasic C18, 5μ, 100×21.2 mm)

Eluents:

1. Low pH chromatography: Solvent A: H₂O+0.1% Formic Acid, pH 1.5, Solvent B: CH₃CN+0.1% Formic Acid

2. High pH chromatography: Solvent A: H₂O+10 mM NH₄HCO₃ +NH₄OH, pH 9.5, Solvent B: CH₃CN

3. Make up solvent:

MeOH+0.1% Formic Acid (for both chromatography type)

Methods: According to the analytical trace the most appropriate preparative chromatography type was chosen. A typical routine was to run an analytical LC-MS using the type of chromatography (low or high pH) most suited for compound structure. Once the analytical trace showed good chromatography a suitable preparative method of the same type was chosen. Typical running condition for both low and high pH chromatography methods were:

Flow rate: 24 ml/min

Gradient: Generally all gradients had an initial 0.4 min step with 95% A+5% B. Then according to analytical trace a 3.6 min gradient was chosen in order to achieve good separation (e.g. from 5% to 50% B for early retaining compounds; from 35% to 80% B for middle retaining compounds and so on)

Wash: 1 minute wash step was performed at the end of the gradient

Re-equilibration: 2.1 minute re-equilibration step was ran to prepare the system for the next run

Make Up flow rate: 1 ml/min

Solvent: All compounds were usually dissolved in 100% MeOH or 100% DMSO

MS running conditions: Capillary voltage: 3.2 kV, Cone voltage: 25 V, Source Temperature: 120° C., Multiplier: 500 V, Scan Range: 125-800 amu, lonisation Mode: ElectroSpray Positive

From the information provided someone skilled in the art could purify the compounds described herein by preparative LC-MS.
XVI. The CEM Discover microwave system was used for all microwave heated chemical reactions. This system is available from CEM <http://www.cemsynthesis.com>; CEM Corporation, P.O. Box 200, Matthews, NC 28106 or CEM Microwave Technology Ltd., 2 Middle Slade, Buckingham Industrial Park MK18 1WA, United Kingdom.
XVII. Terms and abbreviations: Solvent mixture compositions are given as volume percentages or volume ratios. In cases where the NMR spectra are complex, only diagnostic signals are reported. atm: atmospheric pressure; Boc: t-butoxycarbonyl; Cbz: benzyloxycarbonyl; DCM: methylene chloride; DIPEA: diisopropylethylamine; DMF: N;N-dimethyl formamide; DMSO: dimethyl sulfoxide; Et₂O: diethyl ether; EtOAc: ethyl acetate; h: hour(s); HPLC: high pressure liquid chromatography; minute(s): min.; NMR: nuclear magnetic resonance; psi: pounds per square inch; TFA: trifluoroacetic acid; THF: tetrahydrofuran; ACN: acetonitrile.

Experimental Methods

### EXAMPLE 1

### N˜3˜-(1-naplithylmethyl)pyridine-2,3-diamine

General Procedure: Reductive Amination (Method 1)

A mixture of 2,3-diaminopyridine (327 mg, 3.0 mmol), aryl aldehyde (1-naphthaldehyde) (3.0 mmol) and acetic acid (8-10 drops) in dichloromethane (15 ml) were stirred at room temperature for 30 minutes. Sodium triacetoxyborohydride (1.91 g, 9.0 mmol) was added and the mixture stirred at room temperature overnight. The mixture was washed with an equal volume of 10% aqueous potassium carbonate solution, the organic layer separated, the solvent removed in vacuo and the residue purified by column chromatography on silica. Elution with mixtures of ethyl acetate and methanol afforded the product. ¹H NMR (400 MHz, Me₂CO-d₆): δ 4.59 (br s, 1H); 4.80 (d, 2H, J=5); 5.00 (br s, 2H); 6.53 (dd, 1H, J=7.5, 5); 6.78 (dd, 1H, J=7.5, 1); 7.42-7.47 (m, 2H); 7.52-7.58 (m, 3H); 7.85 (d, 1H, J=8); 7.94 (dd, 1H, J=7.5, 2); 8.15-8.17 (m, 1H). m/z (ES) 249 [M⁺].

### 3′-(2-Hydroxy-ethyl)-biphenyl-3-carbaldehyde

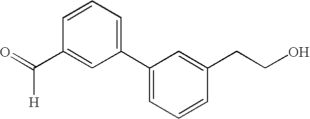

To a mixture of 2-(3-bromo-phenyl)-ethanol (1.34 g, 6.17 mmol) and benzaldehyde-3-boronic acid in toluene (1.3 g, 6.67 mmol) in a mixture of ethanol (5 ml), toluene (5 ml) and 2N sodium carbonate solution in water (5 ml) was added palladium hydroxide 20% wt/wt on carbon (80 mg). The reaction was heated to reflux for 18 hours under nitrogen. The mixture was cooled to room temperature and the solvent removed in vacuo. The resulting material was partitioned between EtOAc and H₂O, dried over MgSO₄ and evaporated to dryness. Material purified by silica chromatography to give the title compound as an oil, 240 mg. ¹H NMR (400 MHz, CDCl₃): δ 3.0 (t, 2H); 3.95 (t, 2H); 7.19-7.55 (m, 5H); 7.63 (t, 1H); 7.88 (d, 1H); 8.12 (s, 1H); 10.12 (s, 1H).

The following additional examples were prepared by reductive amination according to the procedure above: (Method 1)

### EXAMPLE 2

### From 2,3-diaminopyridine and 2-naphthaldehyde

### EXAMPLE 3

### From 2,3-diaminopyridine and 3-phenyl-benzaldehyde

### EXAMPLE 4

### From 2,3-diaminopyridine and 3-pyridin-3-yl-benzaldehyde

### EXAMPLE 5

### From 2,3-diaminopyridine and 3-benzyloxy-benzaldehyde)

### EXAMPLE 6

### From 2,3-diaminopyridine and 3′-(2-hydroxyethyl)-biphenyl-3-carbaldehyde

### EXAMPLE 7

### From 2,3-diaminopyridine and 3-butoxy-benzaldehyde

### EXAMPLE 8

### From 2,3-diaminopyridine and 5-bromo-2-ethoxy-benzaldehyde

### EXAMPLE 9

### From 2,3-diaminopyridine and 9-ethyl-9H-carbazole-3-carbaldehyde

### EXAMPLE 10

### From 2,3-diaminopyridine 2-benzyloxy-5-bromobenzaldehyde

### EXAMPLE 11

### From 2,3-diaminopyridine 5-bromo-2-(2-pyridylmethyloxy)-benzaldehyde

### EXAMPLE 12

### From 6-chloro-pyridine-2,3-diamine and 3′-methoxy-biphenyl-3-carbaldehyde

### EXAMPLE 13

### From 2,3-diaminopyridine and 3′,4′-dimethoxy-biphenyl-3-carbaldehyde

TABLE 1

| | | | | | Molecular | LCMS Rt |
|---|---|---|---|---|---|---|
| Ex. | name | Structure | Method | NMR | Ion (M+) | (min) |
| 1 | N~3~-(1-naphthylmethyl)pyridine-2,3-diamine | 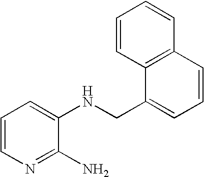 | 1 | 1H NMR (400 MHz,Me2CO-d6): δ 4.59 (br s,1H); 4.80 (d, 2H, J = 5);5.00 (br s, 2H); 6.53 (dd,1H, J = 7.5, 5); 6.78 (dd,1H, J = 7.5, 1); 7.42-7.47(m, 2H); 7.52-7.58 (m, 3H);7.85 (d, 1H, J = 8); 7.94(dd, 1H, J = 7.5, 2); 8.15-8.17 (m, 1H). | 249 | 2.13 (acidic) |
| 2 | N~3~-(2-naphthylmethyl)pyridine-2,3-diamine | 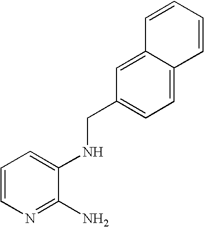 | 1 | 1H NMR (400 MHz,CDCl3): δ 4.60 (s, 2H);6.53 (t, 1H, J = 6.1); 6.68(d, 1H, J = 7.6); 7.04 (d,1H, J = 5.8); 7.46-7.50 (m,3H); 7.79-7.83 (m, 4H). | 249 | 2.98 (basic) |
| 3 | N~3~-(1,1′-biphenyl-3-ylmethyl)pyridine-2,3-diamine | 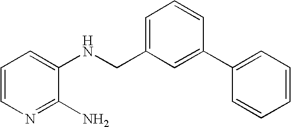 | 1 | ¹H NMR (400M Hz,CDC1₃): δ 4.50 (s, 2H); 6.52(t, 1H, J = 7.6); 6.61 (d, 1H,J = 6.1); 6.69 (d, 1H, J = 5.5); 7.32 (m, 2H); 7.41 (t,2H, J = 7.3); 7.47 (d, 1H, J =7.3); 7.57 (m, 3H); 7.94(br, 2H); 8.08 (s, 1H); 8.75(s, 1H). | 275 | 3.12 (basic) |
| 4 | N~3~-(3-pyridin-3-ylbenzyl)pyridine-2,3-diamine | 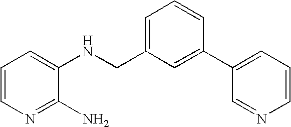 | 1 | ¹H NMR (400 MHz,CDC1₃): δ 3.65 (br s, 1H);4.23 (br s, 2H); 4.38 (d, 2H,J = 4.8); 6.79 (dd as t, 1H, J =6.0); 6.84 (d, 1H, J = 7.5); 7.37 (dd, 1H, J = 4.2, J =7.9); 7.43 (d, 1H, J = 7.3); 7.47 (d, 1H, J = 7.6);7.51 (t, 1H, J = 6.5); 7.59(s, 1H); 7.63 (d, 1H, J = 5.0); 7.86 (d, 1H, J = 7.8);8.60 (d, 1H, J = 4.8); 8.84(s, 1H). | 276 | 1.57 (acidic) |
| 5 | N~3~-[3-(benzyloxy)benzyl]pyridine-2,3-diamine | 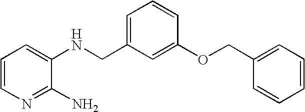 | 1 | ¹H NMR (400 MHz,MeOH-d₄): δ 4.34 (s, 2H);5.08 (s, 2H); 6.54 (dd, 1H, J =5.0, J = 7.9); 6.67 (dd,1H, J = 1.2, J = 7.8); 6.89(dd, 1H, J = 2.0, J = 8.2); 6.98 (d, 1H, J = 7.6); 7.04(s, 1H); 7.24 (t, 1H, J = 8.1); 7.29-7.37 (m, 4H); 7.42 (d, 2H, J = 8.8). | 305 | 2.28 (acidic) |
| 6 | 2-(3′-{[(2-aminopyridin-3-yl)amino]methyl}-1,1′-biphenyl-3-yl)ethanol | 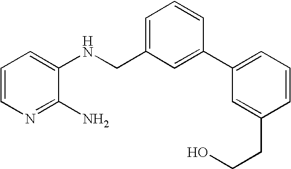 | 1 | ¹H NMR (400 MHz,MeOH-d₄): δ 2.90 (t, 2H,J = 7.0); 3.82 (t, 2H, J =7.1); 4.50 (s, 2H); 6.73 (dd,1H, J = 6.1, J = 7.8); 6.92(d, 1H, J = 8.1); 7.20-7.50(m, 7H); 7.54 (d, 1H, J =7.6); 7.67 (s, 1H). | 319 | 220 (acidic) |
| 7 | N~3~-(3-butoxybenzyl)pyridine-2,3-diamine | 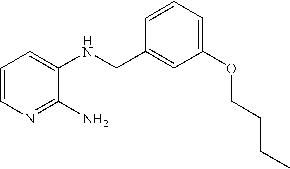 | 1 | ¹H NMR (400 MHz,MeOH-d4): δ 0.99 (t, 3H, J =7.3); 1.51 (sextet, 2H, J = 7.3); 1.74 (quin, 2H, J = 6.4); 3.97 (t, 2H, J = 6.4);4.34 (s, 2H); 6.54 (dd, 1H, J =5.0, J = 7.9); 6.69 (dd, J = 1.3, J = 7.7); 6.80 (dd, 1H, J =2.2, J = 8.6); 6.95 (m, 2H); 7.23 (t, 1H, J = 1.3, J = 5.1). | 271 | 2.30 (acidic) |
| 8 | N~3~-(5-bromo-2-ethoxybenzyl)pyridine-2,3-diamine | 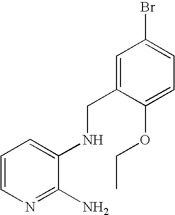 | 1 | ¹H NMR (400 MHz,MeOH-d₄): δ 1.45 (t, 3H, J =6.8); 4.12 (q, 2H, J = 7.1,J = 13.9); 4.33 (s, 2H); 6.56(dd, 1H, J = 5.3, J = 7.8);6.64 (d, 1H, J = 7.8); 6.91(d, 1H, J = 8.6); 7.32-7.40(m, 3H). | 321, 323 | 2.24 |
| 9 | N~3~-[(9-ethyl-9H-carbazol-3-yl)methyl]pyridine-2,3-diamine | 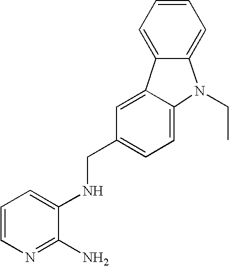 | 1 | ¹H NMR (400 MHz,MeOH-d₄): δ 1.40 (t, 3H, J =7.1); 4.44 (q, 2H, J = 7.3,J = 14.4); 4.53 (s, 2H); 6.63(dd, 1H, J = 5.3, J = 7.6);6.90 (d, 1H, J = 7.6); 7.18(t, 1H, J = 7.6); 7.29 (d, 1H,J = 6.6); 7.42-7.54 (m, 4H);8.17 (d, 1H, J = 7.6); 8.14(s, 1H). | 316 | 2.37 (acidic) |
| 10 | N~3~-[2-(benzyloxy)-5-bromobenzyl]pyridine-2,3-diamine | 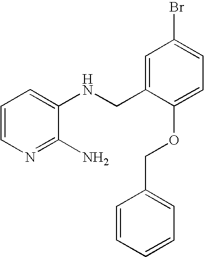 | 1 | ¹H NMR (400 MHz,DMSO-d₆): δ 4.29 (d, 2H, J =5.5); 5.21 (s, 2H); 5.35 (t,1H, J = 5.5); 5.52 (br s,2H); 6.37 (d, 2H, J = 3.5);7.08 (d, 1H, J = 9); 7.29 (t,1H, J = 3.5); 7.33-7.42 (m, 5H); 7.50 (d, 2H, J = 8.5). | 383, 385 | 2.35 (acidic) |
| 11 | N~3~-[5-bromo-2-(pyridin-2-ylmethyoxy)benzyl]pyridine-2,3-diamine | 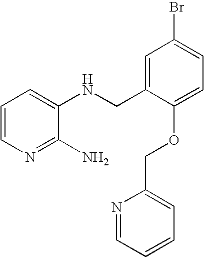 | 1 | ¹H NMR (400 MHz,DMSO-d₆): δ 4.34 (d, 2H,J = 5.5); 5.28 (s, 2H); 5.37(t, 1H, J = 5.5); 5.53 (br s,2H); 6.37-6.46 (m, 2H);7.06 (d, 1H, J = 9); 7.30 (d,1H, J = 5); 7.34-7.39 (m,3H); 7.57 (d, 1H, J = 8);7.83 (td, 1H, J = 7.5, 1.5);8.60 (d, 1H, J = 4.5). | 384, 386 | 2.07 (acidic) |
| 12 | 6-chloro-N~3~-[(3′-methoxy-1,1′-biphenyl-3-yl)methyl]pyridine-2,3-diamine | 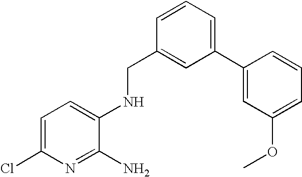 | 1 | ¹H NMR (400 MHz,MeOH-d₄): δ 3.85 (s, 3H);4.42 (s, 2H); 4.58 (s, 2H);6.50 (d, 1H, J = 8.1); 6.68(d, 1H, J = 8.1); 6.92 (dd,1H, J = 2.5, J = 8.3); 7.12(br s, 1H); 7.16 (d, 1H, J =8.3); 7.30-7.54 (m, 3H);7.50 (d, 1H, J = 7.3); 7.61(s, 1H). | 338 | 3.38 (acidic) |
| 13 | N~3~-[(3′,4′-dimethoxy-1,1′-biphenyl-3-yl)methyl]pyridine-2,3-diamine> | 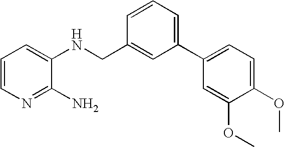 | 1 | ¹H NMR (400 MHz,MeOH-d₃): δ 3.88 (s, 3H);3.90 (s, 3H); 4.47 (s, 2H);6.66 (dd, 1H, J = 5.5, J =7.9); 6.86 (d, 1H, J = 6.5);7.02-7.04 (m, 1H); 7.17-7.19 (m, 2H); 7.28 (d, 1H, J =7.0); 7.35 (d, 1H, J =7.6); 7.41 (t, 3H, J = 7.5);7.51 (d, 1H, J = 7.6); 7.62(s, 1H). | 335 | 2.92 (basic) |

### EXAMPLE 14

### N˜3˜-[3-(1H-indol-6-yl)benzyl]-6-methylpyridine-2,3-diamine

Hydrogenation (Method 2)

6-Methyl-pyridine-2,3-diamine

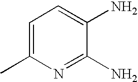

To a stirred solution of 6-methyl-3-nitro-pyridin-2-ylamine (0.27 g, 1.76 mmol) in EtOH (10 mL) and THF (10 mL) was added 10% palladium on carbon (10 mg) and the reaction was purged with hydrogen for 15 minutes and then stirred for a farther 4 hours under a hydrogen atmosphere (1 atm). The hydrogen atmosphere was then removed in vacuo and the resulting mixture filtered through a pad of celite, eluting with EtOAc (50 mL). The solvent was removed under reduced pressure to yield a yellow oil. (0.22 g, 100%). ¹H NMR (400 MHz, MeOH-d₄): δ 2.24 (s, 3H); 6.38 (d, 1H, J=7.6 Hz); 6.85 (d, 2H, J=7.6 Hz).

General Procedure: Reductive Amination (Method 3)

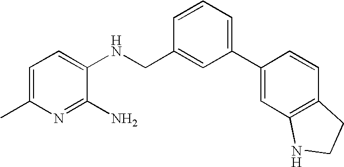

A solution of 6-methyl-pyridine-2,3-diamine (103 mg, 0.84 mmol), 3-(1H-indol-6-yl)-benzaldehyde (prepared by Method 6 described below) (186 mg, 0.84 mmol) and acetic acid (2 drops) in MeOH (10 mL) was stirred for 12 hours. The solvent was removed under reduced pressure and the residue was redissolved in DCM (10 mL). Sodium triacetoxy borohydride (356 mg, 1.68 mmol) was added and the mixture was stirred for 12 hours. To this mixture was added water (30 mL) and the organic layer separated, dried with MgSO₄, filtered and evaporated to dryness. The residue was purified by preparative HPLC to yield the title compound as a yellow solid (45 mg, 15%). ¹H NMR (400 MHz, MeOH-d₄): δ 2.25 (s, 3H); 4.39 (s, 2H); 6.42 (d, 1H, J=7.7); 6.47 (d, 2H, J=2.5); 6.74 (d, 1H, J=7.8); 7.26 (d, 1H, J=J=3.3); 7.30 (t, 2H, J=6.6); 7.40 (t, 1H, J=7.6); 7.55 (d, 1H, J=7.9); 7.60 (d, 2H, J=8.4); 7.68 (s, 1H). m/z (ES) 328 [M⁺].

The following examples were prepared by reductive amination according to the procedure above: (Method 3)

### EXAMPLE 15

### From 6-methyl-3-nitro-pyridin-2-ylamine and 3-phenylbenzaldehyde

### EXAMPLE 16

### from 6-methyl-pyridine-2,3-diamine and 3-(3-pyridyl)benzaldehyde

### EXAMPLE 17

### From 6-methyl-3-nitro-pyridin-2-ylamine and 6-formylindole

### EXAMPLE 18

### from 6-methyl-pyridine-2,3-diamine and 5-(4-fluorophenyl)-pyridine-3-carboxaldehyde

### EXAMPLE 19

### from 6-methyl-pyridine-2,3-diamine and 5-bromo-2-methoxybenzaldehyde

### EXAMPLE 20

### From 6-methyl-3-nitro-pyridin-2-ylamine and 3-phenyl-benzo[c]isoxazole-5-carbaldehyde

TABLE 2

| | | | | | Molecular | LCMS Rt |
|---|---|---|---|---|---|---|
| Ex. | name | Structure | Method | NMR | Ion (M+) | (min) |
| 14 | N~3~-[3-(1H-indol-6-yl) benzyl]-6-methylpyridine-2,3-diamine | 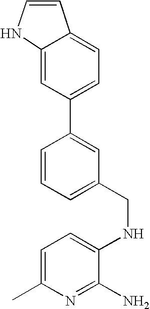 | 3 | ¹H NMR (400 MHz,MeOH-d₄): δ 2.25 (s,3H); 4.39 (s, 2H); 6.42(d, 1H, J = 7.7); 6.47(d, 2H, J = 2.5); 6.74(d, 1H, J = 7.8); 7.26(d, 1H, J = J = 3.3);7.30 (t, 2H, J = 6.6);7.40 (t, 1H, J = 7.6);7.55 (d, 1H, J = 7.9);7.60 (d, 2H, J = 8.4);7.68 (s, 1H). | 328 | 3.05(basic) |
| 15 | N~3~-(1,1′-biphenyl-3-ylmethyl)-6-methylpyridine-2,3-diamine | 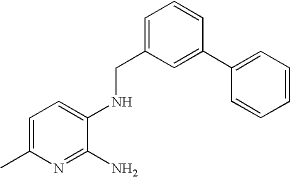 | 3 | ¹H NMR (400 MHz,MeOH-d₄): δ 2.25 (s,3H); 4.33 (s, 2H); 6.26(d, 1H, J = 7.8); 6.56(d, 1H, J = 7.8); 7.24-7.42 (m, 6H); 7.48-7.51 (m, 3H). | 289 | 3.25(basic) |
| 16 | 6-methyl-N~3~-(3-pyridin-3-ylbenzyl)pyridine-2,3-diamine | 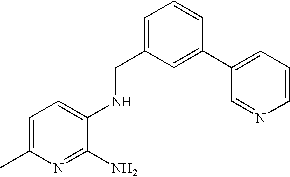 | 3 | ¹H NMR (400 MHz,MeOH-d₄): δ 2.20 (s,3H); 4.43 (s, 2H); 6.40(d, 1H, J = 7.3); 6.70(d, 1H, J = 7.8); 7.52(m, 4H); 7.69 (s, 1H);8.08 (dt, 1H, J = 1.8, J =8.1); 8.51 (dd, 1H, J =1.5, J = 4.8); 8.80(dd, 1H, J = 0.76, J =2.5). | 291 | 2.67(basic) |
| 17 | N~3~-(1H-indol-6-ylmethyl)-6-methylpyridine-2,3-diamine | 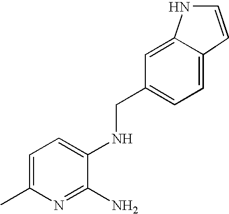 | 3 | ¹H NMR (400 MHz,MeOH-d₄): δ 2.25 (s,3H); 4.41 (s, 2H); 6.40-6.42 (m, 2H); 6.75 (d,1H, J = 7.6); 7.07 (d,1H, J = 7.1); 7.20 (d,1H, J = 2.9); 7.40 (s,1H); 7.52 (d, 1H, J =8.0). | 252 | 2.65(basic) |
| 18 | N~3~-{[5-(4-fluorophenyl)pyridin-3-yl]methyl}-6-methyl-pyridine-2,3-diamine | 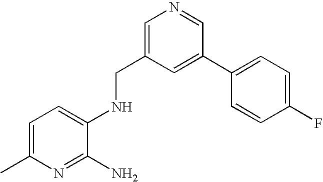 | 3 | ¹H NMR (400 MHz,MeOH-d₄): δ 2.30 (s,3H); 4.50 (s, 2H); 6.48(d, 1H, J = 7.8); 6.82(d, 1H, J = 7.8); 7.25 (t,2H, J = 8.8); 7.70 (m,2H); 8.09 (s, 1H); 8.55(m, 1H); 8.69 (d, 1H, J =2.3). | 309 | 2.78(basic) |
| 19 | N~3~-(5-bromo-2-methoxybenzyl)-6-methylpyridine-2,3-diamine | 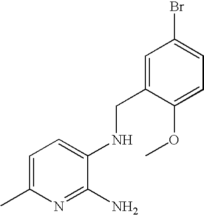 | 3 | ¹H NMR (400 MHz,MeOH-d₄); δ 2.25 (s,3H); 4.27 (s, 2H); 6.38(d, 1H, J = 7.8); 6.58(d, 1H, J = 7.8); 6.90(d, 1H, J = 7.8); 7.33(m, 2H). | 321, 323 | 3.06(basic) |
| 20 | 6-Methyl-N~3~-(3-phenyl-benzo[c]isoxazol-5-ylmethyl)-pyridine-2,3-diamine | 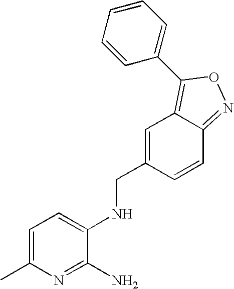 | 3 | ¹H NMR (400 MHz,MeOH-d₄): δ 2.12 (s,3H); 4.28 (s, 2H); 6.28(d, 1H, J = 7.8); 6.63(d, 1H, J = 7.6); 7.35(dd, 1H, J = 1.3, J =9.1); 7.40-7.52 (m,4H); 7.78 (s, 1H); 7.89-7.92 (M, 2H). | 330 | 3.00(basic) |

### EXAMPLE 21

### N˜3˜-[(3′-methoxy-1,1′-biphenyl-3-yl)methyl]-6-methylpyridine-2,3-diamine

General Procedure: Reductive Amination (Method 4)

### N˜3˜-(3-Bromo-benzyl)-6-methyl-pyridine-2,3-diamine

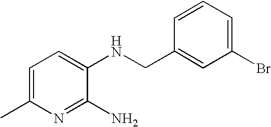

To a stirred solution of 6-methyl-pyridine-2,3-diamine (0.15 g, 1.22 mmol) in DCM (10 mL) was added 3-bromobenzaldehyde (0.14 mL, 1.22 mmol), triethylamine (0.51 ml, 3.66 mmol) and 4 Å molecular sieves followed by sodium triacetoxyborohydride (1.03 g, 4.88 mmol). The reaction was allowed to stir at RT for 16 hours. The reaction was monitored by t.l.c and further equivalents of sodium triacetoxyborohydride were added as required. The mixture was filtered and diluted with DCM, washed with H₂O, dried over MgSO₄ and the solvent removed in vacuo. The residue was purified by column chromatography eluting with 5% MeOH in DCM to give the title compound as a pale yellow oil. (0.116 g, 33%). ¹H NMR (400 MHz, MeOH-d₄): δ 1.99 (s, 3H); 2.31 (s, 3H); 4.36 (s, 2H); 6.4 (d, 1H, J=8.5 Hz); 6.7 (d, 1H, J=7.7 Hz); 7.2 (t, 1H, J=7.8 Hz); 7.3 (d, 1H, J=7.5); 7.4 (d, 1H, J=7.8 Hz); 7.54 (s, 1H). m/z (ES) 292 [M+H].

General Procedure: Suzuki Reaction (Method 5)

### N˜3˜-[(3′-methoxy-1,1′-biphenyl-3-yl)methyl]-6-methylpyridine-2,3-diamine

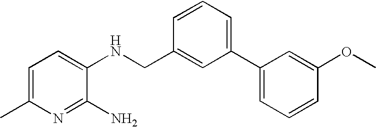

To a degassed solution of N˜3˜-(3-Bromo-benzyl)-6-methyl-pyridine-2,3-diamine (0.1 g, 0.34 mmol), in toluene (1.5 mL) was added bis(tri-tert-butylphosphine)palladium(0) (5 mg) followed by 3-methoxyphenylboronic acid (0.125 g, 0.82 mmol) as a solution in ethanol (1.5 mL). Potassium carbonate (0.282 g, 2.04 mmol) was then added as a solution in H₂O (2 mL) followed by methanol (2 mL). The reaction was heated to 135° C. for 35 minutes in the microwave. The mixture was cooled to room temperature and the solvent removed in vacuo. The resulting material was partitioned between DCM and H₂O, dried over MgSO₄ and evaporated to dryness. Material purified by preparative HPLC to give the title compound as a pale yellow solid. (0.043 g, 39%). ¹H NMR (400 MHz, MeOH-d₄): δ 2.28 (s, 3H); 4.48 (s, 2H); 6.44 (d, 1H, J=7.3); 6.72 (d, 1H, J=7.9); 7.39-7.42 (m, 1H); 7.52-7.55 (m, 3H); 7.66-7.73 (m, 2H); 7.78-7.80 (m, 1H); 8.15-8.17 (m, 1H); 8.35 (s, 1H); 9.22 (s, 1H). m/z (ES) 319 [M+H].

The following additional examples were similarly prepared according to the reductive amination procedure above: (Method 4)

### EXAMPLE 22

### From 2,3-diaminopyridine and 3,8-dimethoxy-2-naphthaldehyde

### EXAMPLE 23

### From 2,3-diaminopyridine and phenylacetaldehyde

### EXAMPLE 24

### From 2,3-diaminopyridine and 2,3-dimethyl-4-methoxybenzaldehyde

### EXAMPLE 25

### From 2,3-diaminopyridine and 3′-hydroxymethyl-biphenyl-3-carbaldehyde

### EXAMPLE 26

### From 2,3-diaminopyridine and 3-(cyclopentyloxy)benzaldehyde

### EXAMPLE 27

### From 2-alkyloxy-5-bromobenzaldehyde

### EXAMPLE 28

### From 2-propargyloxy-5-bromobenzaldehyde

### EXAMPLE 29

### From 2,3-diamino-5-bromopyridine and 3-methoxy-biphenyl-3-carbaldehyde

### EXAMPLE 30

### From 2,3-diaminopyridine and 2-benzyloxy-5-tert-butyl-benzaldehyde

### EXAMPLE 31

### From 2,3-diamino-5-chloropyridine and 3-methoxy-biphenyl-3-carbaldehyde

### EXAMPLE 32

### From 5-bromo-2-(3-pyridylmethyloxy)benzaldehyde

### EXAMPLE 33

### From 2,3-diamino-5-chloropyridine and 3-(pyridyl)benzaldehyde

### EXAMPLE 34

### From 2,3-diaminopyridine and 5-bromo-2-isobutoxy-benzaldehyde

The following examples were prepared by sequential reductive amination and Suzuki coupling according to the procedures above: (Methods 4 and 5)

### EXAMPLE 35

### From 2-chloropyridine-5-boronic acid and 3-bromobenzaldehyde

### EXAMPLE 36

### From 3-methoxypyridine-5-boronic acid and 3-bromobenzaldehyde

### EXAMPLE 37

### From furan-2-boronic acid and 3-bromobenzaldehyde

### EXAMPLE 38

### From 3-formylphenylboronic acid and pyrimidine-5-boronic acid

### EXAMPLE 39

### From 2-methoxy-5-pyridineboronic acid and 3-bromobenzaldehyde

### EXAMPLE 40

### From 3-aminobenzeneboronic acid and 3-bromobenzaldehyde

The following examples were prepared by sequential Suzuki coupling and reductive amination according to the procedures above: (Methods 5 and 4)

### EXAMPLE 41

### From 3-formylphenylboronic acid and 3-chloro-5-propoxy-pyridine

The following examples were prepared by sequential hydrogenation, reductive amination and Suzuki coupling according to the procedures above: (Methods 2, 1 and 5)

### EXAMPLE 42

### From 5-bromo-2-methoxybenzaldehyde and 4-cyanomethylphenyl-boronic acid

### EXAMPLE 43

### From 5-bromo-2-methoxybenzaldehyde and 3-acetamidobenzene-boronic acid

### EXAMPLE 44

### From 5-bromo-2-methoxybenzaldehyde and isoquinoline-4-boronic acid

### EXAMPLE 45

### From 5-bromo-2-methoxybenzaldehyde and 3-methoxyphenylboronic acid

The following examples were prepared by sequential hydrogenation, reductive amination and Suzuki coupling according to the procedures above: (Methods 2, 4 and 5)

### EXAMPLE 46

### From 3-bromobenzaldehyde and isoquinoline-4-boronic acid

### EXAMPLE 47

### From 3-bromobenzaldehyde and 3-aminobenzeneboronic acid

### EXAMPLE 48

### From 3-bromobenzaldehyde and furan-2-boronic acid

### EXAMPLE 49

### From 5-bromo-2-methoxybenzaldehyde and furan-2-boronic acid

TABLE 3

| | | | | | Molecular | LCMS Rt |
|---|---|---|---|---|---|---|
| Ex. | name | Structure | Method | NMR | Ion (M+) | (min) |
| 21 | N~3~-[(3′-methoxy-1,1′biphenyl-3-yl)1-2,3-diamine | 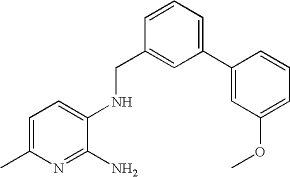 | 4 | ¹H NMR (400 MHz,MeOH-d₄): δ 2.35 (s, 3H);3.86 (s, 3H); 4.48 (s, 2H);6.54 (d, 1H, J = 8.1); 6.90(d, 1H, J = 8.1); 6.93 (dd,1H, J = 2.5, J = 8.3); 7.13(s, 1H); 7.18 (d, 1H, J =9.4); 7.35 (d, 1H, J = 7.7);7.40 (d, 1H, J = 6.8); 7.44(t, 1H, J = 7.6); 7.54 (d, 1H,J = 7.6); 7.63 (s, 1H). | 319 | 3.16 (basic) |
| 22 | N~3~-[(3,8-dimethoxy-2-naphthyl)methyl]pyridine-2,3-diamine | 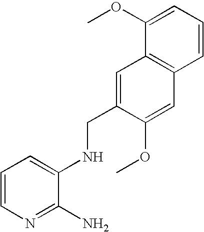 | 4 | ¹H NMR (400 MHz,DMSO-d₆): δ 3.88 (s, 3H);3.96 (s, 3H); 4.38 (d, 2H, J =5.2); 5.08 (t, 1H, J = 4.9);5.55 (d, 2H, J = 7.1); 6.33-6.40 (m, 2H); 6.78 (d, 1H, J =7.2); 7.26 (dd, 1H, J =1.5, J = 4.8); 7.31-7.39(m, 3H); 8.01 (s, 1H). | 310 | 2.96 (basic) |
| 23 | N~3~-(2-phenylethyl)pyridine-2,3-diamine | 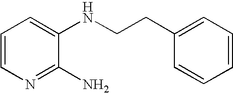 | 4 | ¹H NMR (400 MHz,MeOH-d₄): δ 2.95 (t, 2H, J =7.1); 3.36 (t, 2H, J = 7.8);6.62 (dd, 1H, J = 7.6, J =7.8); 6.83 (dd, 1H, J = 1.3,J = 7.6); 7.19-7.25 (m,1H);7.29-7.39 (m, 5H). | 213 | 2.78 (basic) |
| 24 | N~3~-(4-methoxy-2,3-dimethylbenzyl)pyridine-2,3-diamine | 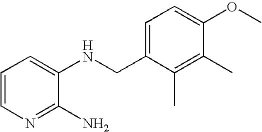 | 4 | ¹H NMR (400 MHz,MeOH-d₄): δ 2.08 (s, 3H);2.17 (s, 3H); 3.7 (s, 3H);4.14 (s, 2H); 6.57(dd, 1H, J =5.6, J = 7.8); 6.65 (d, 1H,J = 8.6); 6.75 (dd, 1H, J =1.0, J = 7.8); 7.02 (d, 1H, J =8.3); 7.16 (dd, 1H, J =1.3, J = 5.6). | 257 | 2.92 (basic) |
| 25 | (3′-{[(2-aminopyridin-3-yl)amino]methyl}-1,1′-biphenyl-3-yl)methanol | 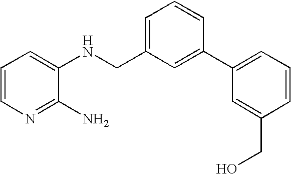 | 4 | ¹ H NMR (400 MHz,MeOH-d₄): δ 4.46(s, 2H);4.68 (s, 2H); 6.64-6.68 (dd,1H, J = 6.1, J = 7.9);6.85-6.87 (dd, 1H, J = 0.9,J = 7.8); 7.20-7.22(dd, 1H, J = 1.3,J = 5.8); 7.33-7.45 (m, 4H);7.45-7.55 (m, 2H); 7.62 (s,1H); 7.66(s, 1H). | 305 | 2.67 (basic) |
| 26 | N~3~-[3-(cyclopentyloxy)benzyl]pyridine-2,3-diamine | 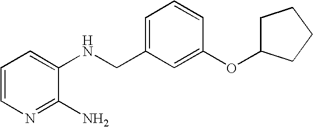 | 4 | ¹H NMR (400 MHz,MeOH-d₄): δ 1.53 (m, 2H);1.66 (m, 4H); 1.78 (m, 2H);4.25 (s, 2H); 4.68 (m, 1H);6.49-6.53 (dd, 1H, J = 5.56, J = 7.51); 6.64-6.68 (m,2H); 6.79-6.82 (m, 2H);7.11 (t, 1H, J-7.8); 7.12 (dd,1H, J = 0.9, J = 5.5). | 284 | 3.11 (basic) |
| 27 | N~3~-[2-(allyloxy)-5-bromobenzyl]pyridine-2,3-diamine | 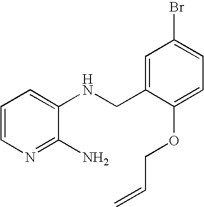 | 4 | ¹H NMR (400 MHz,DMSO-d₆): δ 4.26 (d, 2H, J =5.5); 4.65 (d, 2H, J = 5);5.30 (d, 1H, J = 9); 5.32 (t,1H, J = 5.5); 5.43 (d, 1H, J =14); 5.52 (br s, 2H); 6.07(m, 1H); 6.39 (m, 2H); 7.00(d, 1H, J = 9); 7.29 (t, 1H, J =3.5); 7.33-7.42 (m, 2H). | 333, 335 | 2.30 (acidic) |
| 28 | N~3~-[5-bromo-2-(prop-2-ynyloxy)benzyl]pyridine-2,3-diamine | 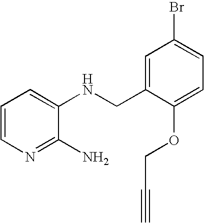 | 4 | ¹H NMR (400 MHz,DMSO-d₆): d 3.62 (t, 1H, J =1.5); 4.26 (d, 2H, J = 5.5);4.90 (s, 2H); 5.32 (t, 1H, J =5.5); 5.50 (br s, 2H); 6.39(m, 2H); 7.08 (d, 1H, J = 9);7.29 (t, 1H, J = 3.5); 7.36(d, 1H, J = 3.5); 7.42 (dd,1H, J = 9, 1.5). | 331, 333 | 2.18 (acidic) |
| 29 | 5-bromo-N~3~-[(3′-methoxy-1,1′-biphenyl-3-yl)methyl]pyridine-2,3-diamine | 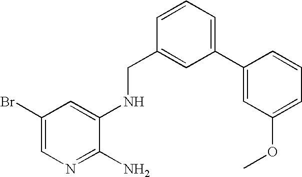 | 4 | ¹ H NMR (400 MHz,MeOH-d₄): δ 3.72 (s, 3H);4.28 (s, 2H); 6.66 (s, 1H);6.79 (dd, 1H, J = 1.8, J =8.0); 7.01 (s, 1H); 7.06 (d,1H, J = 7.6); 7.07-7.20(m, 3H); 7.30 (t, 1H, J =7.5); 7.39 (d, 1H, J = 7.6);7.50 (s, 1H). | 305 | 3.32 (basic) |
| 30 | N~3~-[2-(benzyloxy)-5-tert-butylbenzyl]pyridine-2,3-diamine | 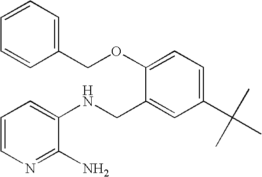 | 4 | ¹H NMR (400 MHz,MeOH-d₄): δ 1.15 (s, 9H);4.28 (s, 2H); 5.06 (s, 2H);6.43-6.46 (m, 1H); 6.62(d, 1H); 6.88 (d, 1H, J =8.3); 7.15 (dd, 1H, J = 2.2, J =8.5); 7.18-7.28 (m, 5H);7.36 (d, 2H, J = 6.7). | 361 | 3.60 (basic) |
| 31 | 5-chloro-N~3~-[(3′-methoxy-1,1′-biphenyl-3-yl)methyl]pyridine-2,3-diamine | 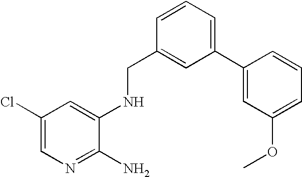 | 4 | ¹H NMR (400 MHz,MeOH-d₄): δ 3.72 (s, 3H);4.29 (s, 2H); 6.55 (d, 1H, J =2.1); 6.77 (dddd, 1H, J =0.7, J = 2.5); 7.00-7.01(m, 1H); 7.06 (dddd, 1H, J =0.7, J = 1.7); 7.14 (d, 1H,J = 2.0); 7.23 (dd, 2H, J =7.6, J = 15.4); 7.30 (t, 1H, J =7.5); 7.40 (d, 1H, J = 7.6);7.50 (s, 1H). | 339 | 2.54 (acidic) |
| 32 | N~3~-[5-bromo-2-(pyridin-3-ylmethoxy)benzyl]pyridine-2,3-diamine | 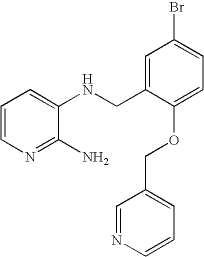 | 4 | ¹H NMR (400 MHz,DMSO-d₆): δ 4.28 (d, 2H, J =5.5); 5.25 (s, 2H; 5.36 (t,1H, J = 5.5); 5.53 (br s,2H); 6.37 (m, 2H); 7.12 (d,1H, J = 9); 7.29 (t, 1H, J =3.5); 7.36 (d, 1H, J = 3.5);7.40-7.47 (m, 2H); 7.93 (dt,1H, J = 7.5, 1.5); 8.56 (m,1H); 8.72 (d, 1H, J = 1.5). | 384, 386 | 2.84 (basic) |
| 33 | 5-chloro-N~3~-(3-pyridin-3-ylbenzyl)pyridine-2,3-diamine | 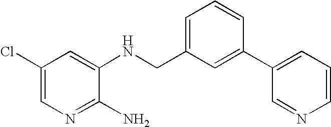 | 4 | ¹H NMR (400 MHz,MeOH-d₄): δ 4.34 (s, 2H);6.54 (d, 1H, J = 2.2); 7.14 (d,1H, J = 2.3); 7.35-7.43 (m,3H); 7.47 (d, 1H, J = 7.3);7.59 (s, 1H); 7.97-8.00 (m,1H); 8.40-8.42 (dd, 1H,J = 1.5, J = 4.7); 8.69 (dd,1H, J = 0.8, J = 2.6). | 311 | 2.56 (basic) |
| 34 | N~3~-(5-bromo-2-isobutoxybenzyl)pyridine-2,3-diamine | 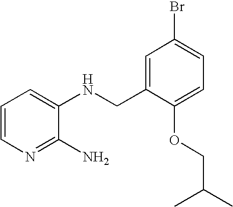 | 4 | ¹H NMR (400 MHz,MeOH-d₄): δ 0.93-0.95 (d,6H, J = 6.9); 1.95-2.05 (m,1H); 3.69-3.70 (d, 2H, J =6.3); 4.24 (s, 2H); 6.47-6.50(dd, 1H, J = 7.9, J = 13.3);6.56-6.58 (dd, 1H, J = 1.6,J = 7.9); 6.77-6.79 (d, 1H,J = 8.8); 7.16-7.18 (dd, 1H,J = 1.6, J = 5.6); 7.21-7.24(dd, 1H, J = 2.8, J = 8.9);7.25-7.26 (m, 1H). | 350 | 3.42 (basic) |
| 35 | N~3~-[3-(6-chloropyridine-3-yl)benzyl]pyridine-2,3-diamine | 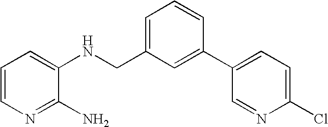 | 4, 5 | ¹H NMR (400 MHz,MeOH-d₄): δ 4.46 (s, 2H); 6.55 (dd, 1H, J = 5.1, J =7.6); 6.75 (dd, 1H, J = 1.5,J = 7.6); 7.34 (dd, 1H, J =1.3, J = 5.1); 7.48 (d, 2H, J =4.5); 7.55-7.60 (m, 2H);7.70 (s, 1H); 8.08 (dd, 1H,J = 2.5, J = 8.3); 8.62 (dd,1H, J = 0.8, J = 2.5). | 310 | 2.84 (basic) |
| 36 | N~3~-[3-(5-methoxypyridin-3-yl)benzyl]pyridine-2,3-diamine | 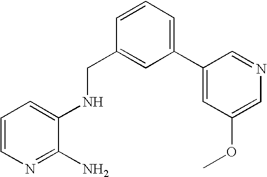 | 4, 5 | ¹H NMR (400 MHz,MeOH-d₄): ^{d} 3.92 (s, 3H);4.44 (s, 2H); 6.53 (dd, 1H,J = 5.4, J = 7.5); 6.72 (dd,1H, J = 1.2, J = 7.9); 7.33(dd, 1H, J = 1.4, J = 5.1); 7.45 (d, 1H, J = 5.1); 7.51-7.56 (m, 2H); 7.66 (s, 1H);8.20 (d, 1H, J = 2.7); 8.36(d, 1H, J = 1.8). | 306 | 2.62 (basic) |
| 37 | N~3~-[3-(2-furyl)benzyl]pyridine-2,3-diamine | 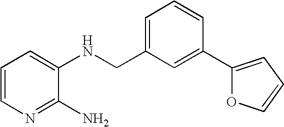 | 4, 5 | ¹H NMR (400 MHz,MeOH-d₄): δ 4.33 (s, 2H);6.42 (dd, 1H, J = 1.7, J =3.3); 6.51 (dd, 1H, J = 5.3,J = 7.8); 7.20-7.23 (m, 2H);7.28 (t, 1H, J = 7.5); 7.45-7.46 (m, 1H: 7.51 (d, 1H, J =7.7); 7.6 (s, 1H). | 265 | 2.93 (basic) |
| 38 | N~3~-(3-pyrimidin-5-ylbenzyl)pyridine-2,3-diamine | 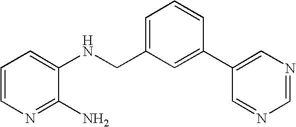 | 4, 5 | ¹H NMR (400 MHz,MeOH-d₄): δ 4.34 (s, 2H);6.54 (d, 1H, J = 2.2);7.14 (d, 1H, J = 2.3); 7.35-7.43 (m, 3H); 7.47 (d, 1H, J =7.3); 7.59 (s, 1H); 7.97-8.00 (m, 1H); 8.40-8.42 (dd,1H, J = 1.5, J = 4.7); 8.69(dd, 1H, J = 0.8, J = 2.6). | 278 | 2.37 (basic) |
| 39 | N~3~-[3-(6-methoxypyridin-3-ylbenzyl]pyrinine-2,3-diamine | 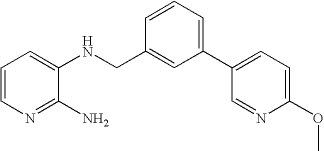 | 4, 5 | ¹H NMR (400 MHz,MeOH-d₄): δ 3.96(s, 3H);4.45 (s, 2H);6.54-6.58 (dd,1H, J = 5.4, J = 7.9); 6.74-6.76 (dd, 1H, J =1.5, J = 7.8);6.87-6.90 (dd, 1H, J = 0.7,J = 8.6); 7.33-7.34 (dd, 1H,J = 1.3, J = 5.1); 7.39-7.50(m, 3H); 7.62 (M, 1H);7.92-7.95 (dd, 1H, J = 2.5,J = 8.6); 8.36 (d, 1H,J = 2.5). | 307 | 2.83 (basic) |
| 40 | N~3~-[(3′-amino-1,1′-biphenyl-3-yl)methyl]pyridine-2,3-diamine | 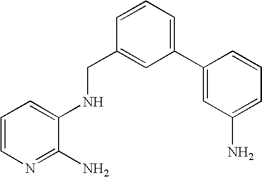 | 4, 5 | ¹H NMR (400 MHz,MeOH-d₄): δ 4.26 (s, 2H);6.42 (dd, 1H, J = 5.0, J =7.9); 6.57-6.61 (m, 2H);6.80 (d, 1H, J = 8.6); 6.85(s, 1H); 7.03 (t, 1H, J =7.6); 7.20-7.26 (m, 3H);7.33 (d, 1H, J = 7.3); 7.47(s, 1H). | 290 | 2.68 (basic) |
| 41 | N~3~-[3-(5-propoxypyridin-3-yl)benzyl]pyridine-2,3-diamine | 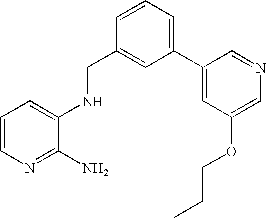 | 5, 4 | ¹H NMR (400 M Hz,MeOH-d₄): δ 1.01 (t, 3H, J =7.5, J = 14.9); 1.79 (q,2H, J = 6.3, J = 14.2); 4.02(t, 2H, J = 6.4, J = 12.9)4.41 (s, 2H); 6.55 (dd, 1H,J = 5.2, J = 7.5); 6.75 (d,1H, J = 9.0); 7.22 (dd, 1H,J = 1.3, J = 5.6); 7.40 (d,2H, J = 6.1); 7.48-7.51 (m,2H); 7.61 (s, 1H); 8.13(d, 1H, J = 2.8); 8.28 (d,1H, J = 1.7). | Not det | Not det |
| 42 | (3′-{[(2-amino-6-methylpyridin-3-yl)amino]methyl}-4′-methoxy-1,1′-biphenyl-3-yl)acetonitrile | 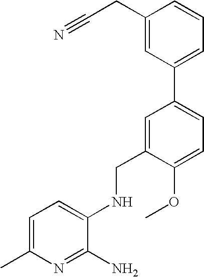 | 2, 1, 5 | ¹H NMR (400 MHz,MeOH-d₄): δ 2.25 (s, 3H);3.91 (s, 2H); 3.94 (s, 3H);4.36 (s, 2H); 6.42 (d, 1H, J =7.8); 6.71 (d, 1H, J = 7.8);7.10 (d, 1H, J = 8.6); 7.38(d, 2H, J = 8.6); 7.51-7.56(m, 4H). | 358 | 2.99 (basic) |
| 43 | N-(3′-{[(2-amino-6-methylpyridin-3-yl)amino]methyl-4′-methoxy-1,1′-biphenyl-3-yl)acetamide | 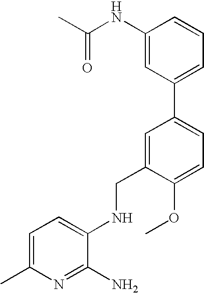 | 2, 1, 5 | 1H NMR (400 MHz,MeOH-d₄): d 2.14 (s, 3H);2.24 (s, 3H); 3.93 (s, 3H);4.35 (s, 2H); 6.42 (d, 1H, J =7.8); 6.71 (d, 1H, J = 7.8);7.07 (d, 1H, J = 8.5); 7.25(d, 1H, J = 7.6); 7.32 (t, 1H,J = 7.9); 7.43-7.46 (m, 1H);7.51 (dd, 1H, J = 2.2, J =8.4); 7.56 (d, 1H, J = 2.2);7.74 (s, 1H). | 376 | 2.72 (basic) |
| 44 | N~3~-(5-isoquinolin-4-yl-2-methoxy-benzyl)-6-methylpyridine-2,3-diamine | 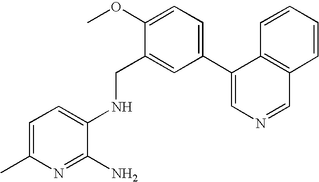 | 2, 1, 5 | ¹H NMR (400 MHz,MeOH-d₄): δ 2.28 (s, 3H);4.01 (s, 3H); 4.45 (s, 2H);6.46 (d, 1H, J = 8.4); 6.68(d, 1H, J = 7.57); 7.20 (d,1H, J = 7.7); 7.45 (m, 2H);7.60 (t, 1H, J = 7.2); 7.67 (t,1H, J = 5.8); 7.72 (d, 1H, J =8.6); 8.13 (d, 1H, J = 8.3);8.29 (s, 1H); 9.17 (s, 1H). | 370 | 2.94 (basic) |
| 45 | N~3~-[(3′,4-dimethoxy-1,1′-biphenyl-3-yl)methyl]-6-methylpyridine-2,3-diamine | 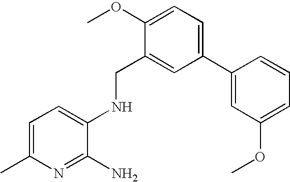 | 2, 1, 5 | ¹H NMR (400 MHz,MeOH-d₆): δ 2.42 (s, 3H);3.84 (s, 3H); 3.94 (s, 3H);4.77 (s, 2H); 6.52 (d, 1H, J =7.5); 6.88 (dd, 1H, J =2.2′ J = 8.3); 7.03 (d, 1H, J =7.9); 7.09 (s, 1H); 7.14(m, 2H); 7.32 (t, 1H, J =7.8); 7.65 (m, 2H); 8.43 (s,1H). | 349 | 2.30 (acidic) |
| 46 | N~3~-(3-isoquinolin-4-ylbenzyl)-6-methylpyridine-2,3-diamine | 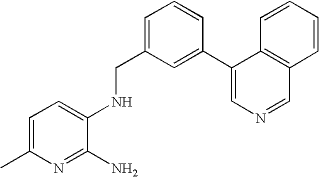 | 2, 4, 5 | ¹H NMR (400 MHz,MeOH-d₄): δ 2.28 (s, 3H);4.48 (s, 2H); 6.44 (d, 1H, J =7.3); 6.72 (d, 1H, J = 7.9);7.39-7.42 (m, 1H); 7.52-7.55 (m, 2H); 7.78-7.80 (m,1H); 8.15-8.17 (m, 1H);8.35 (s, 1H); 9.22 (s, 1H). | 340 | 291 (basic) |
| 47 | N~3~-[(3′-amino-1,1′-biphenyl-3-yl)methyl]-6-methylpyridine-2,3-diamine | 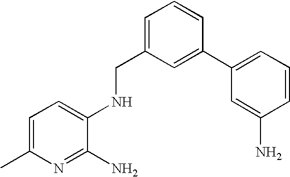 | 2, 4, 5 | ¹H NMR (400 MHz,MeOH-d₄): δ 2.28 (s, 3H);4.41 (s, 2H); 6.44-6.46 (d,1H, J = 7.8); 6.71 (dd, 1H,J = 1.3, J = 7.9); 6.75-6.77 (d,1H, J = 7.8); 6.92-6.94 (d,1H, J = 7.8); 6.98 (m, 1H);7.15-7.19 (t, 1H, J = 7.8);7.33-7.41(m, 2H); 7.47-7.49 (d, 1H, J = 7.4); 7.60 (s,1H). | 305 | 2.74 (basic) |
| 48 | N~3~-[3-(2-furyl)benzyl]-6-methylpyridine-2,3-diamine | 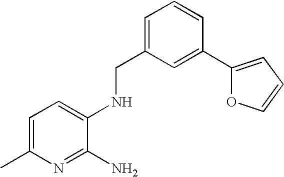 | 2,4,5 | ¹H NMR (400M Hz,MeOH-d₄): δ 2.15 (s, 3H);4.27 (s, 2H); 6.31 (d, 1H,J = 7.1); 6.42 (dd, 1H, J =1.7, J = 3.3); 6.59 (d, 1H, J = 7.8); 6.66 (d, 1H, J = 3.5);7.20 (d, 1H, J = 7.5); 7.27(t, 1H, J = 7.6); 7.45 (s,1H); 7.49 (d, 1H, J = 7.8);7.64 (s, 1H). | 279 | 2.26 (acidic) |
| 49 | N~3~-[5-(2-furyl)-2-methoxybenzyl]-6-methylpyridine-2,3-diamine | 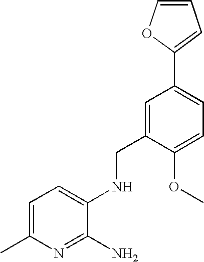 | 2,4,5 | ¹H NMR (400 MHz,MeOH-d₄): δ 2.16 (s, 3H);3.83 (s, 3H); 4.23 (s, 2H);6.32 (d, 1H, J = 7.8); 6.35(dd, 1H, J = 1.8, J = 3.3);6.46 (d, 1H, J = 4.0); 6.94(d, 1H, J = 8.7); 7.38 (s,1H); 7.49 (dd, 1H, J = 1.9,J =8.6); 7.52 (s, 1H). | 309 | 2.23 (acidic) |

### EXAMPLE 50

### N˜3˜-[3-(2-methyl-1,3-benzothiazol-5-yl)benzyl]pyridine-2,3-diamine

General procedure: Suzuki reaction (Method 6)

### 3-(2-Methyl-benzothiazol-5-yl)-benzaldehyde

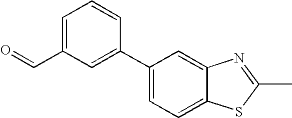

To a stirred suspension of 3-formylphenylboronic acid (0.24 g, 1.61 mmol), 5-bromo-2-methylbenzothiazole (0.35 g, 1.53 mmol) and tetrakis(triphenylphosphine)palladium(0) (5 mg) in DMF (3 mL) was added 2M K₃PO₄ (aq) (1 mL). The reaction was heated to 80° C. in a ReactiVial™ for 2 hours. The mixture was cooled to room temperature and the solvent removed in vacuo. The resulting material was partitioned between DCM and H₂O, dried over MgSO₄ and evaporated to dryness. Material purified by column chromatography eluting with 10% EtOAc in petrol to 20% EtOAc in petrol to give the title compound as a colourless crystalline solid. (0.238 g, 58%). ¹H NMR (400 MHz, MeOH-d₄): δ 2.78 (s, 3H); 7.53-7.56 (m, 2H); 7.8 (d, 1H, J=7.9); 7.83-7.86 (m, 2H); 7.9 (d, 1H, J=1.53 Hz); 8.05 (m, 1H); 9.98 (s, 1H). m/z (ES) 254 [M+H].

N˜3˜-[3-(2-methyl-1,3-benzothiazol-5-yl)benzyl]pyridine-2,3-diamine was prepared in a similar way to Example 50 (reductive amination Method 4). ₁H NMR (400 MHz, MeOH-d₄): δ 2.73 (s, 3H); 4.34 (s, 2H); 6.48 (dd, 1H, J=5.3, J=7.6); 6.68 (dd, 1H, J=1.2, J=7.9); 7.20 (dd, 1H, J=1.2, J=5.2); 7.28-7.53 (dd, 1H, J=1.8, J=8.3); 7.60 (s, 1H); 7.83 (d, 1H, J=8.9); 7.96 (d, 1H, J=1.3). m/z (ES) 346 [M+].

The following examples were prepared by sequential Suzuki coupling and reductive amination according to the procedures above: (Methods 6 and 1)

### EXAMPLE 51

### From 3-formylphenylboronic acid and 6-bromoindole

### EXAMPLE 52

### From 3-methoxyphenylboronic acid and 5-bromosalicyaldehyde

The following examples were prepared by sequential reductive Suzuki coupling and reductive amination according to the procedures above: (Methods 6 and 3)

### EXAMPLE 53

### From 3-formylphenylboronic acid and 3-(4-bromo-phenyl)-1H-pyrazole

### EXAMPLE 54

### From 3-formylphenylboronic acid and 6-bromoindole

### EXAMPLE 55

### From 3-formylphenylboronic acid and ethyl-3-bromobenzoate

### EXAMPLE 56

### From 3-formylphenylboronic acid and 3-bromophenylacetonitrile

### EXAMPLE 57

### From 3-formylphenylboronic acid and 5-(5-bromo-2-thienyl)-1H-pyrazole

The following examples were prepared by sequential hydrogenation, reductive amination and Suzuki coupling according to the procedures above: (Methods 2, 1 and 6)

### EXAMPLE 58

### From 5-bromo-2-methoxybenzaldehyde and indole-6-boronic acid

### EXAMPLE 59

### From 5-bromo-2-methoxybenzaldehyde and 3-aminobenzeneboronic acid

TABLE 4

| | | | | | Molecular | LCMS Rt |
|---|---|---|---|---|---|---|
| Ex. | name | Structure | Method | NMR | Ion (M+) | (min) |
| 50 | N~3~-[3-(2-methyl-1,3-benzothiazol-5-yl)benzyl]pyridine-2,3-diamine | 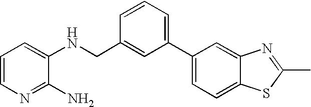 | 6, 4 | ¹H NMR (400 MHz,MeOH-d₄): δ 2.73 (s, 3H);4.34 (s, 2H); 6.48 (dd, 1H,J = 5.3, J = 7.6); 6.68 (dd,1H, J = 1.2, J = 7.9);7.20 (dd, 1H, J = 1.2,J = 5.2); 7.28-7.53 (dd, 1H,J = 1.8, J = 8.3); 7.60 (s,1H); 7.83 (d, 1H, J = 8.9);7.96 (d, 1H, J = 1.3). | 346 | 3.01 (basic) |
| 51 | 5-bromo-N~3~-[3-(1H-indol-6-yl)benzyl]pyridine-2,3-diamine | 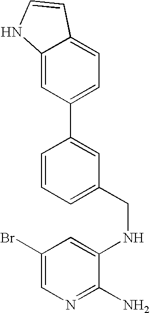 | 6, 1 | ¹H NMR (400 MHz,MeOH-d₄): δ 4.43 (s, 2H);6.47 (d, 1H, J = 3.1); 6.82(d, 1H, J = 2.0); 7.27 (d,1H, J = 3.1); 7.33 (m, 3H);7.43 (t, 1H, J = 7.3); 7.58-7.63 (m, 3H); 7.70 (s, 1H). | 392, 394 | 3.18 (basic) |
| 52 | 3-{[(2-aminopyridin-3-yl)amino]methyl}-3′-methoxy-1,1′-biphenyl-4-ol | 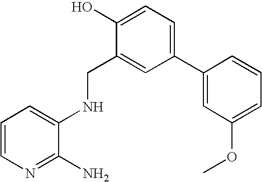 | 6, 1 | ¹H NMR (400 MHz,MeOH-d₄): δ 3.80 (s, 3H);4.40 (s, 2H); 6.59 (dd, 1H,J = 5.3, J = 7.5); 6.83 (t,1H, J = 8.2); 6.89 (d, 1H,J = 8.3); 7.01 (s, 1H);7.07 (d, 1H, J = 7.7); 7.26(t, 1H, J = 7.9); 7.36 (m,2H); 7.48 (s, 1H). | 321 | 2.74 (basic) |
| 53 | N~3~-{[4′-(3-methyl-1H-pyrazol-5-yl)-1,1′-biphenyl-3-yl]methyl}pyridine-2,3-diamine | 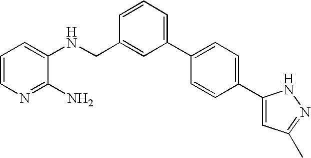 | 6, 3 | ¹H NMR (400 MHz,MeOH-d₄): δ 2.36 (s, 3H);4.52 (s, 2H); 6.47 (s, 1H);6.74 (dd, 1H, J = 5.8,J = 7.9); 6.95 (d, 1H,J = 7.6); 7.27 (d, 1H,J = 5.9); 7.41 (d, 1H,J = 7.6); 7.47 (t, 1H,J = 7.9); 7.60 (d, 1H,J = 7.8); 7.69 (t, 3H,J = 8.5); 7.82 (d, 2H,J = 8.6); 8.42 (s, 1H). | 355 | 2.82 (basic) |
| 54 | N~3~-[3-(1H-indol-6-yl)benzyl]pyridine-2,3-diamine | 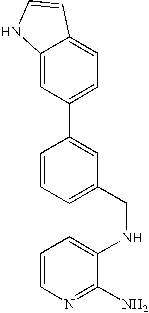 | 6, 3 | ¹H NMR (400 MHz,MeOH-d₄): δ 4.45 (s, 2H);6.46 (d, 1H, J = 3.3); 6.58(dd, 1H, J = 5.3, J = 7.8);6.79 (d, 1H, J = 9.0); 7.27(d, 1H, J = 3.0); 7.30-7.34(m, 3H); 7.40 (t, 1H,J = 7.5); 7.56 (d, 1H,J = 7.6); 7.59-7.61 (m, 2H);7.70 (s, 1H). | 314 | 2.96 (basic) |
| 55 | ethyl 3′-{[(2-aminopyridin-3-yl)amino]methyl}-1,1′-biphenyl-3-carboxylate | 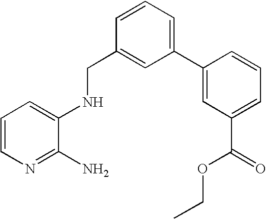 | 6, 3 | ¹H NMR (400 MHz,MeOH-d₄): δ 1.43 (t, 3H,J = 7.1); 4.42 (dd, 2H,J = 7.1, J = 14.5); 4.54 (s,2H); 6.75 (dd, 1H, J = 6.1,J = 7.8); 6.96 (d, 1H,J = 7.8); 7.26 (d, 1H,J = 6.0); 7.44-7.52 (m, 2H);7.56-7.61 (m, 2H); 7.71 (s,1H); 7.88 (d, 1H, J = 8.4);8.02 (d, 1H, J = 7.8); 8.26(s, 1H); 8.39 (s, 1H). | 357 | 2.51 (acidic) |
| 56 | (3′-{[(2-aminopyridin-3-yl)amino]methyl}-1,1′-biphenyl-3-yl)acetonitrile | 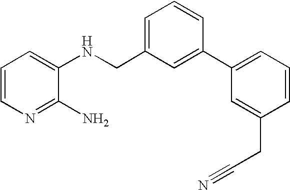 | 6, 3 | ¹H NMR (400 MHz,MeOH-d₄): δ 3.98 (s, 2H);4.53 (s, 2H); 6.76 (dd, 1H,J = 6.1, J = 7.8); 6.97 (d,1H, J = 7.8); 7.24 (d, 1H,J = 6.1); 7.37 (d, 1H,J = 7.8); 7.43 (d, 1H,J = 7.6); 7.46-7.50 (m, 2H);7.50-7.57 (m, 3H); 7.68 (s,1H); 8.40 (s, 1H). | 314 | 2.36 (acidic) |
| 57 | N~3~-{3-[5-(1H-pyrazol-5-yl)thien-2-yl]benzyl}pyridine-2,3-diamine | 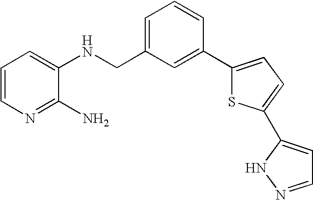 | 6, 3 | ¹H NMR (400 MHz,MeOH-d₄): δ 4.50 (s, 2H);6.61 (d, 1H, J = 2.5); 6.75(1dd, 1H, J = 1.3, J = 6.1);7.35-7.45 (m, 4H); 7.60 (d,1H, J = 7.6); 7.68-7.72 (m,2H); 8.40 (s, 1H). | 347 | 2.34 (acidic) |
| 58 | N~3~-[5-(1H-indol-6-yl)-2-methoxybenzyl]-6-methylpyridine-2,3-diamine | 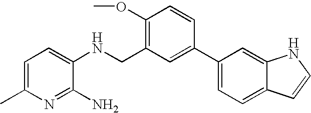 | 2, 1, 6 | ¹H NMR (400 MHz,MeOH-d₄): δ 2.25 (s, 3H);3.92 (s, 3H); 4.35 (s, 2H);6.42 (d, 2H, J = 8.6); 6.74(d, 1H, J = 7.6); 7.05 (d,1H, J = 8.3); 7.20-7.22 (m,2H); 7.51-7.56 (m, 3H);7.60 (d, 1H, J = 2.3). | 358 | 3.12 (basic) |
| 59 | N~3~-[(3′-amino-4-methoxy-1,1′-biphenyl-3-yl)methyl]-6-methylpyridine-2,3-diamine | 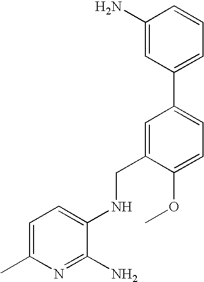 | 2, 1, 6 | ¹H NMR (400 MHz,MeOH-d₄): δ 2.25 (s, 3H);3.93 (s, 3H); 4.34 (s, 2H);6.43 (d, 1H, J = 7.9); 6.64-6.67 (m, 1H); 6.72 (d, 1H,J = 7.6); 6.86 (d, 1H,J = 7.0); 6.91 (t, 1H,J = 1.8); 7.04 (d, 1H,J = 8.3); 7.11 (t, 1H,J = 7.7); 7.47 (dd, 1H,J = 8.6); 7.51 (d, 1H,J = 1.94); 7.51 (d, 1H,J = 1.9). | 334 | 2.8 (basic) |

### EXAMPLE 60

### N˜3˜-(3-bromobenzyl)-6-(morpholin-4-ylcarbonyl)pyridine-2,3-diamine

General Procedure: Ester Hydrolysis (Method 7)

### 6-amino-5-(3-bromo-benzylamino)-pyridine-2-carboxylic acid

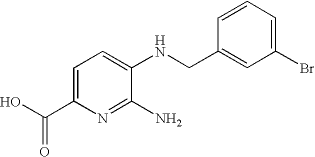

A solution of 6-amino-5-(3-bromo-benzylamino)-pyridine-2-carboxylic acid methyl ester (0.49 g, 1.46 mmol), sodium hydroxide (117 mg, 2.92 mmol), THF (10 mL) and water (4 mL) was heated at reflux for 12 hours. The mixture was cooled, acidified with 1N hydrochloric acid (10 mL). The resulting precipitate, was filtered, washed with water (5 mL) and diethyl ether (5 mL) and dried under reduced pressure to afford a cream solid (385 mg, 83%). ¹H NMR (400 MHz, DMSO-d₆): δ 4.36 (d, 2H, J=5.5 Hz); 5.85 (d, 2H, J=5.3 Hz); 6.16 (s, 2H); 6.48 (d, 1H, J=8.0 Hz); 7.12 (d, 1H, J=8.0 Hz); 7.28 (d, 1H, J=7.8 Hz); 7.36 (d, 1H, J=7.8 Hz); 7.40 (d, 1H, J=7.8 Hz); 7.56 (s, 1H).

General Procedure: Amide Formation (Method 8)

### [6-amino-5-(3-bromo-benzylamino)-pyridin-2-yl]-morpholin-4-yl-methanone

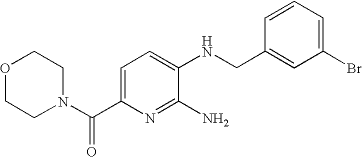

A solution of 6-amino-5-(3-bromo-benzylamino)-pyridine-2-carboxylic acid (0.43 g, 1.34 mmol), morpholine (128 μL, 1.47 mmol), N-ethyl-N′-(3-dimethylaminopropyl)carbodiimide hydrochloride (385 mg, 2.0 mmol), 1-hydroxy-7-azabenzotriazole (273 mg, 2.0 mmol) in DMF (3 mL) was heated at 65° C. for 12 hours. The mixture was cooled, water (20 mL) and DCM (20 mL) were added, the organic layer separated, dried with MgSO₄, filtered and evaporated to dryness. The residue was purified by column chromatography eluting with 5% MeOH in DCM to yield the title compound as a cream foam (228 mg, 40%). ¹H NMR (400 MHz, MeOH-d₄): δ 3.68-3.70 (br, s, 8H); 4.40 (s, 2H); 6.64 (d, 1H, J=8.0 Hz); 6.88 (d, 1H, J=7.8 Hz); 7.28 (d, 1H, J=7.8 Hz); 7.38 (d, 1H, J=7.8 Hz); 7.40 (d, 1H, J=7.8 Hz); 7.58 (s, 1H).

### EXAMPLE 61

### N-(3′-{[(2-aminopyridin-3-yl)amino]methyl}-1,1′-biphenyl-3-yl)propanamide

General Procedure: Acylation (Method 9)

### N-{3′-[(2-amino-pyridin-3-ylamino)-methyl]-biphenyl-3-yl}-propionainide

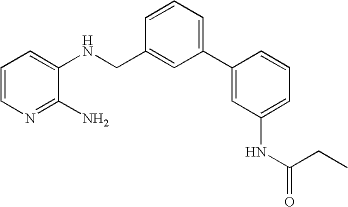

A solution of N˜3˜-(3′-amino-biphenyl-3-ylmethyl)-pyridine-2,3-diamine (0.06 g, 0.21 mmol), triethylamine (33 μL, 0.23 mmol) and propionyl chloride (20 μL, 0.23 mmol) in dry THF was stirred for 12 hours. Water (10 mL) and DCM (10 mL) were added, the organic layer separated, dried MgSO₄, filtered and evaporated to dryness. The residue was purified by preparative HPLC to yield the title compound as a cream solid (18 mg, 26%).

¹H NMR (400 MHz, MeOH-d₄): δ 1.25 (t, 3H, J=7.4); 2.40 (q, 2H, J=7.4); 4.45 (s, 2H); 6.61 (dd, 1H, J=5.6, J=7.5); 6.80 (d, 1H, J=7.9); 7.38 (m, 5H); 7.52 (m, 2H); 7.66 (s, 1H); 7.89 (s, 1H). m/z (ES) 346 [M⁺].

The following examples were prepared by sequential reductive amination, Suzuki coupling and acylation according to the procedures above: (Methods 4, 5 and 9)

### EXAMPLE 62

### From butyryl chloride

### EXAMPLE 63

### From methyl malonyl chloride

### EXAMPLE 64

### From acetoxyacetyl chloride

### EXAMPLE 65

### From 2-(2-methoxyethoxy) acetyl chloride

TABLE 5

| | | | | | Molecular | LCMS Rt |
|---|---|---|---|---|---|---|
| Ex. | name | Structure | Method | NMR | Ion (M+) | (min) |
| 60 | N~3~-(3-bromobenzyl)-6-(morpholin-4-ylcarbonyl)pyridine-2,3-diamine | 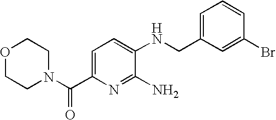 | 7, 8 | ¹H NMR (400 MHz,MeOH-d₄): δ 3.70 (s, 8H);4.42 (s, 2H); 6.64 (d, 1H,J = 8.1); t, J = 7.7); 7.42 (d,1H, J = 7.8); 7.56 (s, 1H). | 390, 392 | 2.61 (basic) |
| 61 | N-(3′-{[(2-aminopyridin-3-yl)amino]methyl}-1,1′-biphenyl-3-yl)propanamide | 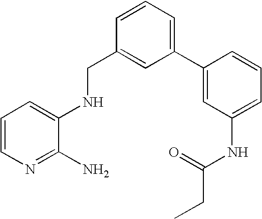 | 4, 5, 9 | ¹H NMR (400 MHz,MeOH-d₄): δ 1.25 (t, 3H),J = 7.4); 2.40 (q, 2H, J = 7.4);4.45 (s, 2H); 6.61 (dd, 1H,J = 5.6, J = 7.5); 6.80 (d, 1H,J = 7.9); 7.38 (m, 5H); 7.52(m, 2H); 7.66 (s, 1H); 7.89 (s,1H). | 346 | 2.76 (basic) |
| 62 | N-(3′-{[(2-aminopyridin-3-yl)1biphenyl-3-yl)butanamide | 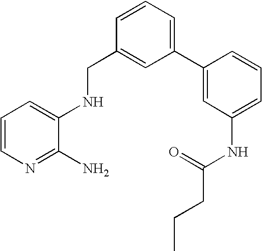 | 4, 5, 9 | ¹H NMR (400 MHz,MeOH-d₄): δ 1.03 (t, 3H,J = 7.4); 1.76 (sext, J = 7.3);2.39 (t, 2H, J = 7.3); 4.55 (s,2H); 6.61 (dd, 1H, J = 5.6,J = 7.5); 6.80 (d, 1H, J = 7.9);7.38 (m, 5H); 7.52 (t, 2H,J = 6.8); 7.66 (s, 1H); 7.89 (s,1H). | 360 | 2.90 (basic) |
| 63 | methyl 3-[(3′-{[(2-aminopyridin-3-yl)amino]methyl}-1,1′-biphenyl-3-yl)amino]-3-oxopropanoate | 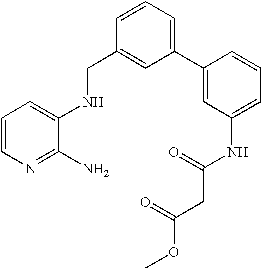 | 4, 5, 9 | ¹H NMR (400 MHz,MeOH-d₄): δ 3.33 (s, 2H);3.78 (s, 3H); 4.51 (s, 2H);6.76 (t, 1H, J = 6.1); 6.96 (d,1H, J = 7.0); 7.24 (d, 1H,J = 6.0); 7.44 (m, 5H); 7.56(d, 1H, J = 7.8); 7.67 (s, 1H);7.9 (s, 1H); 8.40 (s, 1H). | 390 | 2.70 (basic) |
| 64 | 2-[(3′-{[(2-aminopyridin-3-yl)amino]methyl}-1,1′-biphenyl-3-yl)amino]-2-oxoethyl acetate | 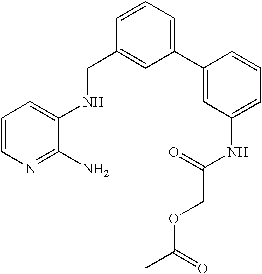 | 4, 5, 9 | ¹H NMR (400 MHz,MeOH-d₄): δ 2.20 (s, 3H);4.51 (s, 2H); 4.73 (s, 2H);6.77 (dd, 1H, J = 6.0,J = 8.1); 6.98 (d, 1H, J = 7.8);7.23 (dd, 1H, J = 1.3,J = 6.0); 7.40-7.51 (m, 5H);7.57 (d, 1H, J = 3.8); 7.67 (s,1H); 7.94 (s, 1H). | 390 | 2.73 (basic) |
| 65 | N-(3′-{[(2-aminopyridin-3-yl)amino]methyl}-1,1′-biphenyl-3-yl)-2-(2-methoxyethoxy)acetamide | 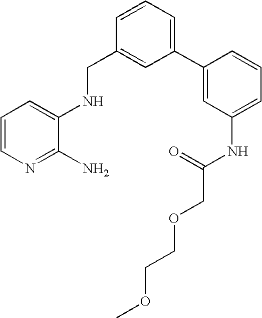 | 4, 5, 9 | ¹H NMR (400 MHz,MeOH-d₄): δ 3.46 (s, 3H);3.66-3.68 (m, 2H); 3.78-3.81(m, 2H); 4.16 (s, 2H); 4.51 (s,2H); 6.74 (dd, 1H, J = 6.0,J = 7.9); 6.94 (d, 1H, J = 7.3);7.25 (d, 1H, J = 5.8); 7.41-7.48 (m, 4H); 7.57 (d, 2H,J = 7.0); 7.68 (s, 1H); 7.95 (s,1H); 8.45 (s, 1H). | 406 | 2.72 (basic) |

General procedure: Alkylation (Method 10)

### 3-Chloro-5-propoxy-pyridine

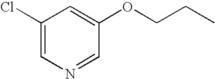

To a solution of 5-chloro-3-pyridinol (1 g, 7.7 mmol) in DMF (16 mL) was added potassium carbonate (3.18 g, 23 mmol) and the reaction heated to 60° C. 1-Chloropropane (2 mL, 23 mmol) was then added and the reaction allowed to stir at 60° C. for 16 hours. The mixture was cooled to room temperature and the solvent removed in vacuo. The resulting material was partitioned between EtOAc and H₂O, the organics washed with brine, dried over MgSO₄ and evaporated to dryness. Material purified by column chromatography eluting with 10% EtOAc in petrol to give the title compound as a pale yellow liquid. (0.231 g, 18%) ¹H NMR (400 MHz, MeOH-d₄): δ 1.06 (t, 3H, J=5.5); 1.80-1.89 (m, 2H); 4.03 (t, 2H, J=6.4); 7.49 (t, 1H, J=2.3); 8.13(d, 1H, J=1.98); 8.19 (d, 1H, J=2.8).

The following examples were prepared by sequential Suzuki coupling, reductive amination and alkylation according to the procedures above: (Methods 5, 1 and 10)

### EXAMPLE 66

### From 4-hydroxy-3′-methoxy-biphenyl-3-carbaldehyde and 2-picolyl chloride

The following examples were prepared by sequential Suzuki coupling, alkylation and reductive amination according to the procedures above: (Methods 5, 10 and 1)

### EXAMPLE 67

### From 4-hydroxy-3′-methoxy-biphenyl-3-carbaldehyde and benzyl bromide

The following examples were prepared by sequential alkylation, reductive amination and Suzuki coupling according to the procedures above: (Methods 10, 1 and 5)

### EXAMPLE 68

### From 5-bromosalicyaldehyde and 2-dimethylaminoethyl chloride

TABLE 6

| | | | | | Molecular | LCMS Rt |
|---|---|---|---|---|---|---|
| Ex. | name | Structure | Method | NMR | Ion (M+) | (min) |
| 66 | N~3~-{[3′-methoxy-4-(pyridin-2-ylmethoxy)-1,1′-biphenyl-3-yl]methyl}pyridine-2,3-diamine | 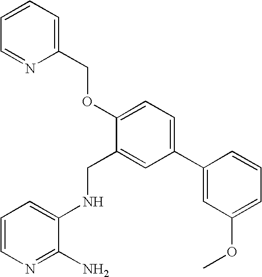 | 5, 1, 10 | ¹H NMR (400 MHz, MeOH-d₄):_{d} 3.83 (s, 3H); 4.55 (s, 2H); 5.33(s, 2H); 6.73 (t, 1H, J = 6.2); 6.87(d, 1H, J = 8.5); 6.98 (d, 1H,J = 7.8); 7.05 (s, 1H); 7.10 (d, 1H,J = 7.1); 7.14 (d, 1H, J = 8.4);7.25-7.32 (m, 2H); 7.40 (t, 1H,J = 5.2); 7.53 (d, 1H, J = 8.5);7.58 (s, 1H); 7.62 (d, 1H, J = 8.0);7.87 (t, 1H, J = 6.5); 8.46 (s, 1H);8.59 (d, 1H, J = 5.0). | 421 | 3.05 (basic) |
| 67 | N~3~-{[4-(benzyloxy)-3′-methoxy-1,1′-biphenyl-3-yl]methyl}pyridine-2,3-diamine | 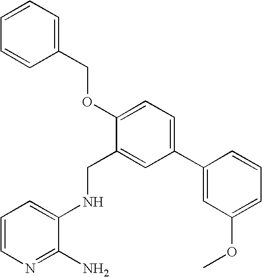 | 5, 10, 1 | ¹H NMR (400 MHz, MeOH-d₄):δ 3.81 (s, 3H); 4.47 (s, 2H); 5.25(s, 2H); 6.56 (dd, 1H, J = 5.0,J = 7.5); 6.74 (dd, 1H, J = 1.5,J = 7.8); 6.84 (dd, 1H, J = 1.25,J = 8.1); 7.01 (t, 1H, J = 1.8);7.07-7.10 (m, 1H); 7.15 (d, 1H,J = 8.5); 7.28 (t, 1H, J = 7.9);7.33-7.35 (m, 2H); 7.39 (t, 2H,J = 7.1); 7.47-7.52 (m, 3H); 7.55(d, 1H, J = 2.5). | 411 | 3.47 (basic) |
| 68 | N~3~-[2-[2-(dimethylamino)ethoxy]-5-(1H-indol-6-yl)benzyl]pyridine-2,3-diamine | 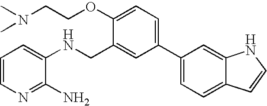 | 10, 1, 5 | ¹H NMR (400 MHz, MeOH-d₄):δ 2.37 (s, 6H); 2.84 (t, 2H,J = 5.3); 4.18 (t, 2H, J = 5.3); 4.40(s, 2H); 6.44 (dd, 1H, J = 0.7,J = 3.0); 6.58 (dd, 1H, J = 5.0,J = 7.6); 6.80 (dd, 1H, J = 1.2,J = 7.6); 7.02 (d, 1H, J = 8.6);7.20-7.23 (m, 2H); 7.33 (dd, 1H,J = 1.2, J = 5.0); 7.50-7.61 (m,4H). | 401 | 3.53 (basic) |

General procedure : Reduction (Method 11)

### (6-Amino-5-nitro-pyridin-2-yl)methanol

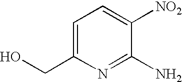

To a solution of 6-amino-5-nitro-pyridine-2-carboxylic acid methyl ester (0.5 g, 2.5 mmol) in Toluene (10 mL) and THF (5 mL) was added lithium aluminium hydride (0.19 g, 5.0 mmol) and the reaction stirred at RT for 16 hours. To the resulting mixture was added EtOAc dropwise (20 mL) followed by H₂O (20 mL). The organic solvents were then removed under reduced pressure. The residue was portioned between DCM and H₂O, the organic layer separated, dried over MgSO₄ and evaporated to dryness. Material purified by column chromatography eluting with 1:1 EtOAc/petrol to give the title compound as a pale brown solid (74 mg, 17%). ¹H NMR (400 MHz, MeOH-d₄): δ 4.58 (s, 2H); 6.91 (d, 1H, J=8.6); 8.47 (d, 1H, J=8.6).

The following example was prepared by sequential reduction, hydrogenation and reductive amination according to the procedures above: (Methods 11, 2 and 3)

### EXAMPLE 69

### From 6-amino-5-nitro-pyridine-2-carboxylic acid methyl ester and 3-bromobenzaldehyde (Methods 11, 2 and 3)

The following example was prepared by sequential reduction, hydrogenation, reductive amination and Suzuki coupling according to the procedures above: (Methods 11, 2, 3 and 5)

### EXAMPLE 70

### From 6-amino-5-nitro-pyridine-2-carboxylic acid methyl ester and pyridyl-3-boronic acid

### (6-Chloro-3-nitro-pyridin-2-yl)-(4-methoxy-benzyl)-amine

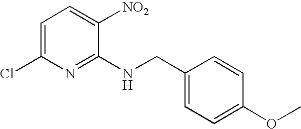

To a stirred solution of 2,6, dichloro-3-nitropyridine (3.0 g, 18.6 minol) and sodium carbonate (4.9 g, 46.6 mmol) in EtOH (30 mL) was added 4-methoxybenzylamine (3.6 mL, 27.9 mmol) and the reaction stirred for 12 hours at room temperature. The resulting mixture was diluted with water (40 mL) and extracted with DCM (2×30 mL). The organic layer was dried over MgSO₄, filtered and evaporated to dryness. The residue was purified by column chromatography eluting with a mixture of EtOAc and petrol-ether to yield the product as yellow solid (2.34 g, 42%). ¹H NMR (400 MHz, MeOH-d₄): δ 3.85 (s, 3H); 4.75 (d, 2H, J=5.6); 6.65 (dd, 1H, J=1.0, J=8.6); 6.90 (d, 2H, J=8.6); 7.33 (d, 2H, J=8.4); 8.37 (dd, 1H, J=1.0, J=8.6); 8.56 (s, 1H).

### (4-Methoxy-benzyl)-(3-nitro-6-phenoxy-pyridin-2-yl)-amine

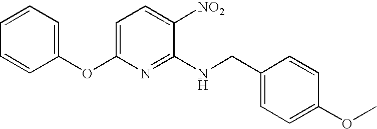

To a stirred solution of (6-chloro-3-nitro-pyridin-2-yl)-(4-methoxy-benzyl)-amine (0.5 g, 1.6 mmol) and phenol (0.48 g, 5.18 mmol) in dry THF (5 mL) was added sodium hydride (60% dispersion in oil, 207 mg, 5.18 mmol) and the reaction stirred for 12 hours at room temperature. The resulting mixture was diluted with water (20 mL) and extracted with DCM (2×20 mL). The organic layer was dried over MgSO₄, filtered and evaporated to dryness to yield the product as yellow oil (0.6 g, 100%). ¹H NMR (400 MHz, MeOH-d₄): δ 3.80 (s, 3H); 4.40 (d, 2H, J=5.8); 6.25 (dd, 1H, J=0.8, J=8.8); 6.80 (d, 2H, J=8.1); 6.85 (d, 1H, J=8.6); 6.95 (d, 2H, J=8.4); 7.15 (d, 2H, J=7.8); 7.45 (t, 2H, J=7.6); 8.41 (d, 1H, J=10.1); 8.85 (s, 1H).

General procedure for deprotection of a PMB group (Method 12):

### 3-Nitro-6-phenoxy-pyridin-2-ylamine

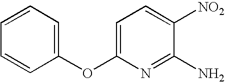

A solution of (4-Methoxy-benzyl)-(3-nitro-6-phenoxy-pyridin-2-yl)-amine (0.6 g, 1.7 mmol) and trifluoroacetic acid (2 mL) in toluene (5 mL) was heated at 100° C. for 2 hours. The resulting mixture was cooled to room temperature, diluted with water (20 mL) and extracted with DCM (2×20 mL). The organic layer was dried over MgSO₄, filtered and evaporated to dryness to yield the product as yellow oil (0.24 g, 61%). ¹H NMR (400 MHz, MeOH-d₄): δ 4.60 (s, 2H); 6.25 (dd, 1H, J=1.3, J=9.1); 7.15 (dd, 2H, J=1.0, J=7.6); 7.30 (m, 1H); 7.45 (t, 2H, J=8.3); 8.45 (dd, 1H, J=1.5, J=9.1).

### 6-Phenoxy-pyridine-2,3-diamine

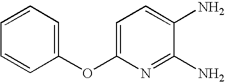

To a stirred solution of 3-Nitro-6-phenoxy-pyridin-2-ylamine (0.24 g, 1.0 mmol) in MeOH (10 mL) and EtOAc (5 mL) was added 10% palladium on carbon (10 mg) and the reaction was purged with hydrogen for 15 minutes and then stirred for a further 12 hours under a hydrogen atmosphere (1 atm). The hydrogen atmosphere was then removed in vacuo and the resulting mixture filtered through a pad of celite, eluting with EtOAc (30 mL). The solvent was removed under reduced pressure to yield the product as a tan crystalline solid (0.12 g, 58%)

¹H NMR (400 MHz, MeOH-d₄): δ 6.05 (d, 1H, J=7.8); 6.83 (d, 1H, J=7.8); 6.92-7.0 (m, 3H); 7.25 (t, 2H, J=8.6).

General alkylation procedure (Method 13):

### N˜3˜-Benzyl-6-phenoxy-pyridine-2,3-diamine

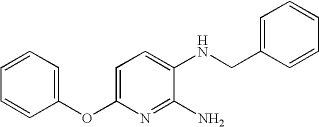

To a solution of 6-phenoxy-pyridine-2,3-diamine (50 mg, 0.25 mmol) in acetonitrile (4.5 mL) and DIPEA (0.5 mL) was added benzyl bromide (46 μL, 0.37 mmol) and the reaction heated at 100° C. for 48 hours. The resulting material was partitioned between EtOAc and H₂O, the organics washed with brine, dried over MgSO₄ and evaporated to dryness. Material purified by preparative HPLC to yield the title compound as brown oil (18 mg, 25%).

¹H NMR (400 MHz, DMSO-d₆): δ 4.25 (d, 2H, J=5.6); 5.20 (s, 1H); 5.75 (s, 1H); 6.00 (d, 1H, J=7.83); 6.60 (d, 1H, J=8.3); 6.88 (dd, 1H, J=1.0, J=8.8); 7.00 (t, 1H, J=6.6); 7.30 (m, 8H).

### 5-Bromo-2-(2-pyrrolidin-1-yl-ethoxy)-benzaldehyde

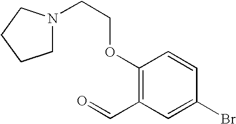

To a solution of 5-bromosalicylaldehyde (0.5 g, 2.48 mmol) in THF (7 mL) was added potassium carbonate (1.03 g, 7.4 mmol), after stirring at room temperature for 15 minutes, 1-(2-chloroethyl)pyrrolidine hydrochloride (0.63 g, 3.7 mmol) was added and the reaction heated to 80° C. for 12 hours. The mixture was cooled to room temperature and the solvent removed in vacuo. The resulting material was partitioned between EtOAc and H₂O, the organics washed with brine, dried over MgSO₄ and evaporated to dryness to yield the title compound as a brown oil. (0.67 g, 100%).

¹H NMR (400 MHz, CDCl₃): δ 1.86-1.92 (br m, 4H); 2.90 (br s, 4H); 3.12-3.16 (br m, 2H); 4.34 (s, 2H, J=4.8); 6.86 (d, 1H, J=8.8); 7.57 (dd, 1H, J=2.8, J=9.1); 7.85 (d, 1H, J=2.5); 10.29 (s, 1H).

### N˜3˜-[5-Bromo-2-(2-pyrrolidin-1-yl-ethoxy)-benzyl]-pyridine-2,3-diamine

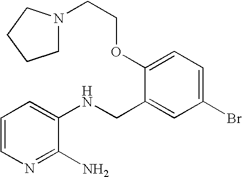

A solution of 5-bromo-2-(2-pyrrolidin-1-yl-ethoxy)-benzaldehyde (0.67 g, 2.48 mmol), 2,3-diaminopyridine (0.27 g, 2.48 mmol) and acetic acid (2 drops) in MeOH (15 mL) was stirred for 48 hours. The solvent was removed under reduced pressure and the residue was redissolved in DCM (15 mL). Sodium triacetoxy borohydride (1.05 g, 4.96 mmol) was added and the mixture was stirred for 12 hours. To this mixture was added water (40 mL) and extracted with DCM (2×30 mL), the combined organic layers washed with water (2×30 mL), dried with MgSO₄, filtered and evaporated to dryness to yield the title compound which wash used directly in the following step. ¹H NMR (400 MHz, MeOH-d₄): δ 1.90-1.98 (m, 4H); 3.18-3.30 (m, 4H); 3.50 (t, 2H, J=5.0); 4.34-4.40 (m, 4H); 6.62-6.70 (m, 2H); 6.79 (dd, 1H, J=1.5, J=7.8); 6.97-7.06 (m, 1H); 7.27-7.30 (m, 1H); 7.41-7.47 (m, 1H).

The following examples were prepared by the Suzuki method described above: (Method 5)

### EXAMPLE 71

### from N˜3-[5-Bromo-2-(2-pyrrolidin-1-yl-ethoxy)-benzyl]-pyridine-2,3-diamine and pyridine-3-boronic acid

### EXAMPLE 72

### from N˜3˜-[5-Bromo-2-(2-pyrrolidin-1-yl-ethoxy)-benzyl]-pyridine-2,3-diamine and indole-6-boronic acid

The following example was prepared by the alkylation method described above: (Method 13)

### EXAMPLE 73

### from 6-phenoxy-pyridine-2,3-diamine and benzyl bromide (6-Chloro-5-cyano-pyridin-2-ylmethyl)-triphenylphosphonium bromide

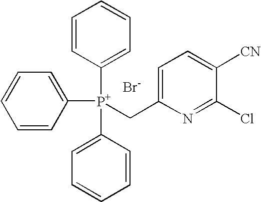

A mixture of the 2-chloro-6-methyl-3-pyridine (6.0 g, 39.4 mmol), N-bromosuccinimide (7.7 g, 43.3 mmol) and AIBN (0.56 g) in anhydrous benzene (100 ml) were heated at reflux for 48 hours. The mixture was cooled to room temperature and then chilled in an ice bath for 30 minutes with vigourous stirring. The insoluble material was removed by filtration and the solvent removed in vacuo. The resulting solid was redissolved in acetonitrile (100 ml), triphenylphosphine (10.3 g, 39.4 mmol) was added and the mixture was refluxed for 24 hours. Upon cooling the solid material was collected by suction filtration, washed with acetonitrile and sucked dry to afford the title compound as a white solid (6.7 g, 35%).

¹H NMR (400 MHz, DMSO-d₆): δ 5.60-5.66 (m, 2H); 7.54 (d, 1H, J=7.8); 7.70-7.92(m, 15H); 8.42 (d, 1H, J=8.1).

### 6-[2-(3-Bromo-phenyl)-vinyl]-2-chloro-nicotinonitrile

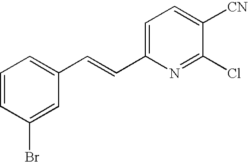

Potassium tert-butoxide (1.67 g, 14.9 mmol) was added to a vigourously stirred suspension of (6-Chloro-5-cyano-pyridin-2-ylmethyl)-triphenylphosphorium bromide (6.7 g, 13.6 mmol) in anhydrous THF (70 ml) and the mixture stirred at room temperature for 30 minutes. To resulting solution solution was added 3-bromobenzaldehyde (2.6 g, 14.2 mmol) and the mixture was refluxed for 12 hours. Upon cooling to room temperature the solvent was removed in vacuo and the resulting solid redissolved in DCM (50 mL) and cooled on ice, the resulting precipitate was filtered and washed with MeOH (30 mL) to yield the product as a cream solid (3.98 g, 90%). ¹H NMR (400 MHz, DMSO-d₆): δ 7.40 (t, 1H, J=7.8); 7.52 (d, 1H, J=16.1); 7.58 (d, 1H, J=8.1); 7.72-7.84 (m, 3H); 8.00 (s, 1H); 8.46 (d, 1H, J=7.8).

### 6-[2-(3-Bromo-phenyl)-vinyl]-2-(4-methoxy-benzyl-amino)-nicotinonitrile

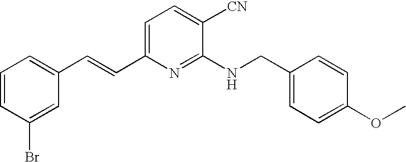

A solution of 6-[2-(3-Bromo-phenyl)-vinyl]-2-chloro-nicotinonitrile (1.0 g, 3.13 mmol) and 4-methoxybenzylamine (1.5 mL) in toluene (2.5 mL) were heated at 100° C. in a sealed tube for 12 hours. The resulting mixture was diluted with water (40 mL) and extracted with DCM (2×30 mL). The organic layer was dried over MgSO₄, filtered and evaporated to dryness. The residue was purified by column chromatography eluting with a mixture of EtOAc and petrol-ether to yield the product as yellow solid (1.0 g, 76%). ¹H NMR (400 MHz, DMSO-d₆): δ 3.70 (s, 3H); 4.59 (d, 2H, J=5.6); 6.77 (d, 1H, J=7.6); 6.88 (d, 2H, J=8.6); 7.27 (d, 1H, J=15.7); 7.36-7.41 (m, 3H); 7.53 (d, 1H, J=8.1); 7.60 (s, 1H); 7.63-7.68 (m, 2H); 7.88-7.92 (m, 2H).

### 6-[2-(3-Bromo-phenyl)-vinyl]-2-(4-methoxy-benzylamino)-nicotinonitrile

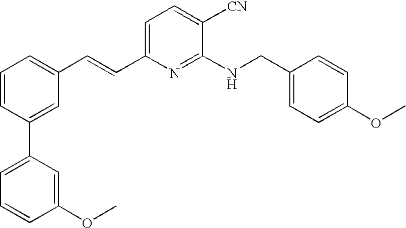

To a degassed solution of 6-[2-(3-Bromo-phenyl)-vinyl]-2-(4-methoxy-benzyl-amino)-nicotinonitrile (1.0 g, 2.38 mmol) in toluene (5 mL) was added bis(tri-tert-butylphosphine)palladium(0) (5 mg) followed by 3-methoxyphenylboronic acid (0.72 g, 4.76 mmol) as a solution in ethanol (5 mL). Potassium carbonate (0.66 g, 4.76 mmol) was then added as a solution in H₂O (8 mL) followed by methanol (5 mL). The reaction was heated to 135° C. for 30 minutes in the microwave. The mixture was cooled to room temperature and the solvent removed in vacuo. The resulting material was partitioned between DCM and H₂O, dried over MgSO₄ and evaporated to dryness, the material was used directly in the next step. ¹H NMR (400 Hz, DMSO-d₆): δ 3.70 (s, 3H); 3.85 (s, 3H); 4.62 (br s, 2H); 6.78 (d, 1H, J=7.8); 6.88 (d, 2H, J=7.3); 6.98 (d, 1H, J=8.1); 7.14-7.20 (m, 1H); 7.24-7.34 (m, 3H); 7.38-7.42 (m, 2H); 7.49-7.54 (m, 1H); 7.62-7.68 (m, 2H); 7.72-7.78 (m, 1H); 7.88-7.94 (m, 2H).

### 2-(4-Methoxy-benzylamino)-6-[2-(3′-methoxy-biphenyl-3-yl)-ethyl]-nicotinonitrile

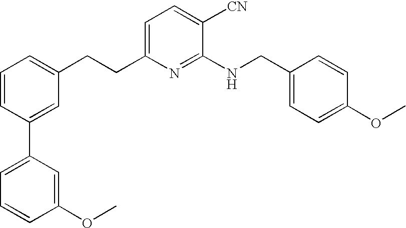

To a stirred solution 6-[2-(3-Bromo-phenyl)-vinyl]-2-(4-methoxy-benzylamino)-nicotinonitrile (1.2 g, 2.38 mmol) in EtOH (25 mL) and THF (10 mL) was added 20% wt palladium hydroxide on carbon (30 mg) and the reaction was purged with hydrogen for 15 minutes and then stirred for a further 12 hours under a hydrogen atmosphere (1 atm). The hydrogen atmosphere was then removed in vacuo and the resulting mixture filtered through a pad of celite, eluting with EtOAc (60 mL). The solvent was removed under reduced pressure and the residue purified by column chromatography to yield the product as colourless oil (0.64 g, 60%) for the 2 steps. ¹H NMR (400 MHz, MeOH-d₄): δ 2.94-3.10 (m, 4H); 3.70 (s, 3H); 3.84 (s, 3H); 4.60 (s, 2H); 6.40 (d, 1H, J=8.3); 6.80 (d, 2H, J=8.3); 6.92 (d, 1H, J=8.3); 6.98 (s, 1H); 7.08 (d, 2H, J=7.8); 7.15 (s, 1H); 7.24-7.40 (m, 5H); 7.60 (d, 1H, J=8.0).

The following example was prepared by the deprotection method described above: (Method 12)

### EXAMPLE 74

### from 2-(4-Methoxy-benzylamino)-6-[2-(3′-methoxy-biphenyl-3-yl)-ethyl]-nicotinonitrile and trifluoroacetic acid

### EXAMPLE 75

### N˜3˜-(1-Biphenylen-2-yl-ethyl)-pyridine-2,3-diamine

General Procedure: Alkylation (Method 14)

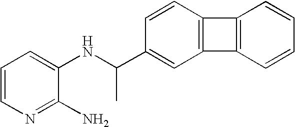

α-methyl-2-biphenylenemethanol was prepared by sodium borohydride reduction of the commercially available ketone in ethanol and was used without further purification. α-methyl-2-biphenylenemethanol (39 mg) was suspended in DCM (1 ml) and phosphorous tribromide added cautiously drop-wise at room temperature under nitrogen. After stirring for 45 minutes the reaction was quenched with water and the mixture extracted with DCM. The organic layer was dried over MgSO₄, filtered, 2,3-diaminopyridine (22 mg) added, and the mixture evaporated to dryness. A 9:1 mixture of MeCN and DIPEA was added to the residues (2 ml), and the mixture heated in a microwave vessel at 180° C. for 2 minutes in a CEM microwave instrument. The cooled reaction was evaporated to dryness and the residues purified by preparative HPLC to yield the product (18 mg). ¹H NMR (400 MHz, CDCl₃): δ 1.60 (d, 3H, J=6.8); 4.16 (q, 1H, J=6.6, J=13.1); 4.68 (s, 1H); 6.45-6.55 (m, 5H); 6.6-6.7 (m, 4H); 6.9 (br d, 1H, J=6.8); 7.95-8.15 (br m, 2H).

### EXAMPLE 76

### N˜3˜-(1,1′-biphenyl-4-ylmethyl)pyridine-2,3-diamine

General Procedure: Alkylation (Method 15)

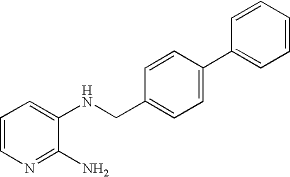

A solution of pyridine-2,3-diamine (50 mg, 0.46 mmol), 4-phenylbenzylchloride (92 mg, 0.45 mmol) and diisopropylethylamine (0.3 ml) in MeCN (3 mL) was heated at 180° C. in a microwave reactor for 2 min. The solvent was removed under reduced pressure and the residue was purified by preparative HPLC to yield the title compound as a beige solid (15 mg, 12%). ¹H NMR (400M Hz, CDCl₃): δ 4.40 (s, 2H); 6.42-6.53 (m, 2H); 6.90 (m, 1H); 7.22-7.32 (m, 5H); 7.43 (t, 4H, J=8 Hz); 7.93 (m, 2H); 13.51 (br, 1H).

The following examples were prepared by sequential reductive amination and Suzuki reaction (Method 4 and Method 18 as outlined below)

### EXAMPLE 77

### From 2-benzyloxy-5-bromobenzaldehyde and indole-6-boronic acid

### EXAMPLE 78

### From 5-bromo-2-(2-pyridylmethyloxy)benzaldehyde and indole-6-boronic acid

### EXAMPLE 79

### From 2-benzyloxy-5-bromobenzaldehyde and pyridine-3-boronic acid

### EXAMPLE 80

### From 5-bromo-2-(3-pyridylmethyloxy)benzaldehyde and indole-6-boronic acid

### EXAMPLE 81

### From 5-bromo-2-(4-pyridylmethyloxy)benzaldehyde and indole-6-boronic acid

TABLE 7

| | | | | | Molecular | LCMS Rt |
|---|---|---|---|---|---|---|
| Ex. | name | Structure | Method | NMR | Ion (M+) | (min) |
| 69 | {6-amino-5-[(3-bromobenzyl)amino]pyridin-2-yl}methanol | 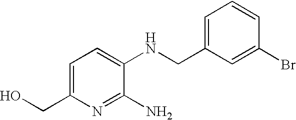 | 11, 2, 3 | ¹H NMR (400 MHz,MeOH-d₄): δ 4.32 (s, 2H);4.40 (s, 2H); 6.57-6.62 (m,2H); 7.16-7.21 (m, 1H);7.30-7.36 (m, 2H); 7.51 (s,1H). | 308 | 2.06 (acidic) |
| 70 | {6-amino-5-[(3-pyridin-3-ylbenzyl)amino]pyridin-2-yl}methanol | 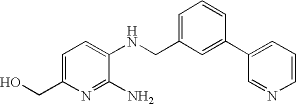 | 11, 2, 3,5 | ¹H NMR (400 MHz,MeOH-d₄): δ 4.53 (s, 2H);4.54 (s, 2H); 6.70 (d, 1H,J = 7.9); 6.92 (d, 1H,J = 7.9); 7.50-7.64 (m, 4H);7.73 (s, 1H); 8.1-8.14 (m,1H), 8.54 (dd, 1H,J = 4.92, J = 1.51); 8.8 (m,1H). | 306 | 3.22 (acidic) |
| 71 | N~3~-[5-pyridin-3-yl-2-(2-pyrrolidin-1-ylethoxy)benzyl]pyridine-2,3-diamine | 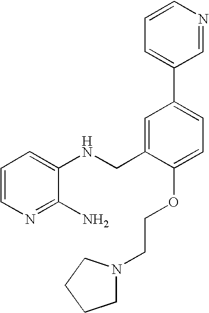 | 5 | ¹H NMR (400 MHz,MeOH-d₄): δ 1.85 (s, 4H);2.95 (s, 4H); 3.33 (m, 2H);4.32 (t, 2H, J = 5.3); 4.45(s, 2H); 6.60 (dd, 1H,J = 5.1, J = 7.6); 6.81 (dd,1H, J = 7.6); 7.18 (d, 1H,J = 8.3); 7.35 (dd, 1H,J = 1.5, J = 5.3); 7.45 (m,1H); 7.60 (m, 2H); 8.00 (m,1H); 8.45 (dd, 1H, J = 1.5,J = 4.8); 8.72 (dd, 1H,J = 0.8, J = 2.3). | 390 | 2.87 (basic) |
| 72 | N~3~-[5-(1H-indol-6-yl)-2-(2-pyrrolidin-1-ylethoxy)benzyl]pyridine-2,3-diamine | 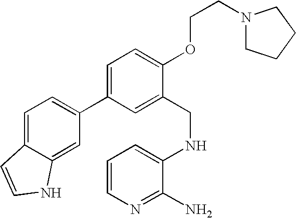 | 5 | ¹H NMR (400 MHz,MeOH-d₄): δ 2.00 (m, 4H);3.40 (m, 4H); 3.60 (t, 2H,J = 4.8); 4.40 (t, 2H,J = 4.8); 4.45 (s, 2H); 6.45(dd, 1H, J = 0.8, J = 3.0);6.76 (m, 1H); 9.96 (dd, 1H,J = 1.0, J = 7.8); 7.10 (d,1H, J = 8.6); 7.20 (m, 3H);7.60 (m, 4H). | 428 | 3.20 (basic) |
| 73 | N~3~-benzyl-6-phenoxypyridine-2,3-diamine | 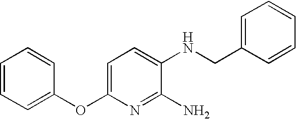 | 13 | ¹H NMR (400 MHz,DMSO-d₆): δ 4.25 (d, 2H,J = 5.6); 5.20 (s, 1H); 5.75(s, 1H); 6.00 (d, 1H,J = 7.83); 6.60 (d, 1H,J = 8.3); 6.88 (dd, 1H,J = 1.0, J = 8.8); 7.00 (t,1H, J = 6.6); 7.30 (m, 8H). | 291 | 3.31 (basic) |
| 74 | 2-amino-6-[2-(3′-methoxy-1,1′-biphenyl-3-yl)ethyl]nicotinonitrile | 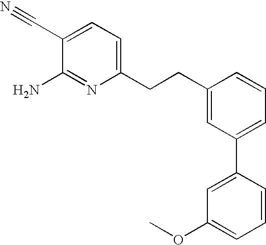 | 12 | ¹H NMR (400 MHz,MeOH-d₄): δ 2.93 (m, 2H);3.00 (m, 2H); 3.82 (s, 3H);6.58 (d, 1H, J = 8.8); 6.85(s, 2H); 6.94 (d, 1H,J = 8.3); 7.14 (s, 1H); 7.18(t, 2H, J = 9.3); 7.35 (m,2H); 7.47 (m, 2H); 7.75 (d,1H, J = 7.8). | 330 | 3.46 (basic) |
| 75 | N~3~-(1-biphenylen-2-ylethyl)pyridine-2,3-diamine | 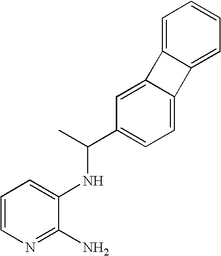 | 14 | ¹H NMR (400 MHz,CDCl₃): δ 1.60 (d, 3H,J = 6.8); 4.16 (q, 1H,J = 6.6, J = 13.1); 4.68 (s,1H); 6.45-6.55 (m, 5H);6.6-6.7 (m, 4H); 6.9 (br d,1H, J = 6.8); 7.95-8.15 (brm, 2H). | 286 | 3.13 (basic) |
| 76 | N~3~-(1,1′-biphenyl-4-ylmethyl)pyridine-2,3-diamine | 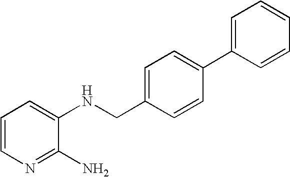 | 15 | ¹H NMR (400 MHz,CDCl₃): δ 4.40 (s, 2H);6.42-6.53 (m, 2H); 6.90 (m,1H); 7.22-7.32 (m, 5H);7.43 (t, 4H, J = 8 Hz); 7.93(m, 2H); 13.51 (br, 1H). | 275 | 2.26 (acidic) |
| 77 | N~3~-[2-(benzyloxy)-5-(1H-indol-6-yl)benzyl]pyridine-2,3-diamine | 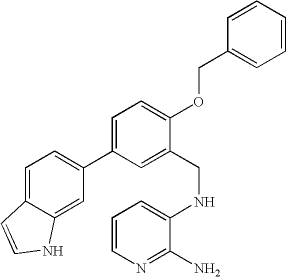 | 4, 18 | ¹H NMR (400 MHz,DMSO-d₆): δ 4.38 (d, 2H,J = 5.5); 5.26 (s, 2H); 5.35(t, 1H, J = 5.5); 5.54 (br s,2H); 6.37-6.41 (m, 2H);6.52 (d, 1H, J = 7.5); 7.18(d, 2H, J = 8.5); 7.27 (dd,1H, J = 5, 1.5); 7.32-7.36(m, 2H); 7.41 (t, 2H,J = 70); 7.50-7.59 (m, 6H);11.10 (br s, 1H). | 420 | 2.63 (acidic) |
| 78 | N~3~-[5-(1H-indol-6-yl)-2-(pyridin-2-ylmethoxy)benzyl]pyridine-2,3-diamine | 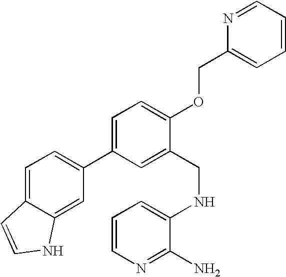 | 4, 18 | ¹H NMR (400 MHz,DMSO-d₆): δ 4.43 (d, 2H,J = 5.5); 5.32 (s, 2H); 5.37(t, 1H, J = 5.5); 5.55 (br s,2H); 6.41 (br s, 2H); 6.58(d, 1H, J = 7.5); 7.15-7.22(m, 2H); 7.28 (d, 1H,J = 4); 7.33-7.38 (m, 2H);7.50 (s, 2H); 7.56 (d, 1H,J = 8); 7.61 (s, 2H); 7.84(td, 1H, J = 7.5, 1.5); 8.61(d, 1H, J = 4); 11.11 (s,1H). | 421 | 2.23 (acidic) |
| 79 | N~3~-[2-(benzyloxy)-5-pyridin-3-ylbenzyl]pyridine-2,3-diamine | 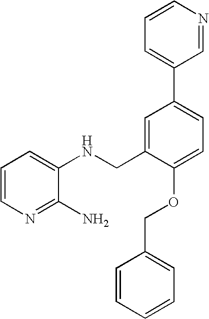 | 4, 18 | ¹H NMR (400 MHz,DMSO-d₆): δ 4.37 (d, 2H,J = 5.5); 5.28 (s, 3H); 5.53(br s, 2H); 6.39 (dd, 1H,J = 7.5, 5); 6.53 (d, 1H,J = 7); 7.23 (d, 1H,J = 8.5); 7.28 (d, 1H,J = 4); 7.35 (d, 1H,J = 7.5); 7.39-7.45 (m, 3H);7.53 (d, 2H, J = 7); 7.61(dm, 1H, J = 5); 7.64 (d,1H, J = 1.5); 7.91-7.94(dm, 1H, J = 7.5); 8.50 (dd,1H, J = 5, 1.5); 8.80 (d,1H, J = 1.5). | 382 | 2.02 (acidic) |
| 80 | N~3~-[5-(1H-indol-6-yl)-2-(pyridin-3-ylmethoxy)benzyl]pyridine-2,3-diamine | 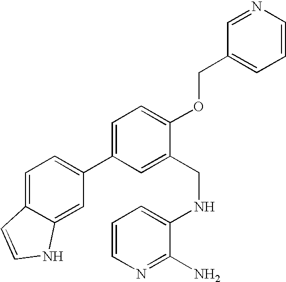 | 4, 18 | ¹H NMR (400 MHz,DMSO-d₆): δ 4.36 (d, 2H,J = 5.5); 5.31 (s, 2H); 5.36(t, 1H, J = 5.5); 5.56 (br s,2H); 6.36-6.42 (m, 2H);6.50 (d, 1H, J = 7.5); 7.18-7.22 (m, 2H); 7.27 (dd, 1H,J = 5, 1.5); 7.34 (t, 1H,J = 2.5); 7.42-7.46 (m, 1H);7.50-7.59 (m, 4H); 7.94-7.97 (dm, 1H, J = 8); 8.56(dd, 1H, J = 4.5, 1.5); 8.75(d, 1H, J = 1.5); 11.13 (brs, 1H). | 421 | 2.09 (acidic) |
| 81 | N~3~-[5-(1H-indol-6-yl)-2-(pyridin-4-ylmethoxy)benzyl]pyridine-2,3-diamine | 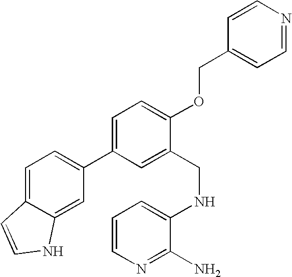 | 4, 18 | ¹H NMR (400 MHz,DMSO-d₆): δ 4.43 (d, 2H,J = 5.5); 5.33 (s, 2H); 5.38(t, 1H, J = 5.5); 5.58 (br s,2H); 6.39-6.42 (m, 2H);6.56 (d, 1H, J = 8.5); 7.12(d, 1H, J = 8.5); 7.19 (dd,1H, J = 8, 1.5); 7.28 (dd,1H, J = 4.5, 1.5); 7.33 (t,1H, J = 2.5); 7.51-7.53 (m,4H); 7.56 (d, 1H, J = 8.5);7.61 (d, 1H, J = 2.5); 8.59(dd, 2H, J = 4.5, 1.5);11.13 (br s, 1H). | 421 | 1.92 (acidic) |

Preparation of phosphonium salt C (Scheme 2)

A mixture of 2-amino-6-methylpyridine (21.6 g, 0.2 mol) and phthalic anhydride (29.6 g, 0.2 mol) were stirred and held at 190° C. for 1 hour and then cooled to room temperature to afford the phthalimide derivative A (45.5 g, 96%) as a pale yellow solid. ¹H NMR (400 MHz, DMSO-d₆): δ 2.52 (s, 3H); 7.37 (d, J=7, 1H); 7.40 (d, J=7, 1H); 7.90-7.95 (m, 3H); 7.97-8.03 (m, 2H).

A mixture of the phthalimide (23.8 g, 0.1 mol), N-bromosuccinimide (19.0 g, 0.11 mol) and AIBN (1.2 g) in anhydrous benzene (400 ml) were stirred and held at reflux for 5 hours. The mixture was cooled to room temperature and then chilled in an ice bath for 30 minutes with vigourous stirring. The insoluble material was removed by filtration and the solvent removed in vacuo. The resulting solid crude bromide B was dissolved in acetonitrile (400 ml), triphenylphosphine (26.2 g, 0.1 mol) was added and the mixture was stirred and held at reflux for 16 hours. Upon cooling the solid material was collected by suction filtration, washed with acetonitrile and sucked dry to afford the product C (36.75 g, 63%) as a colourless solid. ¹H NMR (400 MHz, DMSO-d₆): δ 5.54 (d, J=18, 2H); 7.43 (m, 2H); 7.62-7.68 (m, 6H); 7.78-7.85 (m, 9H); 7.95-8.02 (m, 5H).

General Procedure: Wittig Reaction (Method 16)

Potassium tert-butoxide (130 mg, 1.16 mmol) was added to a vigourously stirred suspension of phosphonium salt C (580 mg, 1.0 mmol) in anhydrous tetrahydrofuran (10 ml) and the mixture stirred at room temperature for 10-15 minutes. To the bright orange solution was added aryl aldehyde (1.0 mmol) and the mixture stirred and held at reflux for 3 hours. Upon cooling to room temperature the solvent was removed in vacuo and the residue was partitioned between dichloromethane and water. The organic extracts were separated, the solvent removed in vacuo and the residue purified by column chromatography on silica. Elution with mixtures of petroleum ether and ethyl acetate afforded the product (50-80%).

General Procedure: Phthalimide Deprotection (Method 17)

Hydrazine hydrate (0.2 ml) was added to a stirred mixture of phthalimide derivative (1.0 mmol) and ethanol (20 ml) and the mixture stirred and held a reflux for 3-5 hours. Upon cooling to room temperature the mixture was filtered, the solvent removed in vacuo and the residue was partitioned between dichloromethane and water. The organic extracts were separated, the solvent removed in vacuo and the residue purified by column chromatography on silica. Elution with mixtures of petroleum ether and ethyl acetate afforded the product (70-90%).

General Procedure: Suzuki Reaction (Method 18)

A mixture of aryl bromide (0.4 mmol), aryl boronate or aryl (pinocol)boronate (0.6 mmol), potassium carbonate (276 mg, 2.0 mmol) and Pd(P^{t}Bu₃)₂ (5-10 mg, catalyst) in a mixture of MeOH, EtOH, H₂O and PhMe (1:1:1:1; 4 ml) were stirred and held at 135° C. for 30 minutes under the influence of microwave irradiation (30 W). Upon cooling the mixture was filtered and the organic solvent removed in vacuo. The residue was partitioned between dichloromethane and water, the organic extracts were separated, the solvent is removed in vacuo and the residue purified by column chromatography on silica. Elution with mixtures of petroleum ether and ethyl acetate afforded the product (70-90%).

General Procedure: Hydrogenation (Method 19)

A vigourously stirred mixture of alkene (0.5 mmol) and 10% Pd/C (30 mg) in methanol (5 ml) was reduced under an atmosphere of hydrogen for 4-16 hours. The catalyst was removed by filtration, solvent removed in vacuo and the residue purified by column chromatography on silica. Elution with mixtures of petroleum ether and ethyl acetate afforded the product (70-90%).

The following examples were prepared by sequential Wittig reaction, hydrogenation and phthalimide deprotection according to the procedures outlined above: (Methods 16, 19 and 17)

### EXAMPLE 82

### From 6-formylindole

### EXAMPLE 83

### From 2-naphthaldehyde

### EXAMPLE 84

### From 3,8-dimethoxy-2-naphthaldehyde

### EXAMPLE 85

### From 3-(3-pyridyl)benzaldehyde

The following examples were prepared by sequential Wittig reaction, phthalimide deprotection, Suzuki coupling and hydrogenation according to the procedures outlined above. (Methods 16, 17, 18 and 19)

### EXAMPLE 86

### From 3-bromobenzaldehyde and 3-methoxy-5-(pinacolboranato)-pyridine

### EXAMPLE 87

### From 3-bromobenzaldehyde and 3-methoxyphenylboronic acid

### EXAMPLE 88

### From 3-bromobenzaldehyde and 2-furylboronic acid

### EXAMPLE 89

### From 3-bromobenzaldehyde and 3,4-diniethoxyphenylboronic acid

The following examples were prepared by sequential Wittig reaction, Suzuki coupling and hydrogenation according to the procedures outlined above: (Methods 16, 18 and 19)

### EXAMPLE 90

### From 3-bromobenzaldehyde and indole-6-boronic acid

### EXAMPLE 91

### From 5-bromo-2-(2-pyridylmethyloxy)benzaldehyde and indole-6-boronic acid

### EXAMPLE 92

### From 5-bromo-2-(2-pyridylmethyloxy)benzaldehyde and indole-6-boronic acid

General Procedure: Indole Chlorination (Method 20)

A solution of the indole (0.8 mmol) in methanol (10 ml) was stirred at 0° C., N-chlorosuccinimide (0.96 mmol) was added and the mixture stirred and allowed to warm to room temperature over 2 hours. The solvent was removed in vacuo and the residue purified by column chromatography on silica. Elution with diethyl ether afforded the product (40-60%).

The following examples were prepared by indole chlorination according to the procedure outlined above: (Method 20)

### EXAMPLE 93

### From 6-(2-(2-amino-6-pyridyl)ethyl)indole

TABLE 8

| | | | | | Molecular | LCMS Rt |
|---|---|---|---|---|---|---|
| Ex. | name | Structure | Method | NMR | Ion (M+) | (min) |
| 82 | 6-[2-(1H-indol-6-yl)ethyl]pyridin-2-amine | 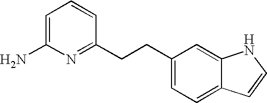 | 16, 19,17 | ¹H NMR (400 MHz,DMSO-d₆): δ 2.79 (t, 2H,J = 7); 2.98 (t, 2H, J = 7);5.82 (br s, 2H); 6.26 (d, 1H,J = 8); 6.35-6.37 (m, 2H);6.74 (dd, 1H, J = 8, 1.5); 7.20(s, 1H); 7.23-7.27 (m, 2H);7.42 (d, 1H, J = 8); 10.90 (brs, 1H). | 237 | 2.77 (basic) |
| 83 | 6-[2-(2-naphthyl)ethyl]pyridin-2-amine | 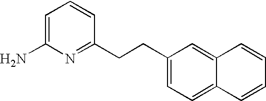 | 16, 19,17 | ¹H NMR (400 MHz,DMSO-d₆): δ 2.87 (t, 2H,J = 7.5); 3.09 (t, 2H, J = 7.5);5.84 (br s, 2H); 6.27 (d, 1H,J = 7.5); 6.38 (d, 1H, J = 7.5);7.25 (t, 1H, J = 7.5); 7.40-7.50 (m, 3H); 7.71 (s, 1H);7.82-7.87 (m, 3H). | 248 | 2.12 (acidic) |
| 84 | 6-[2-(3,8-dimethoxy-2-naphthyl)ethyl]pyridin-2-amine | 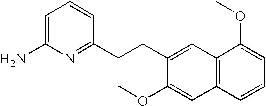 | 16, 19,17 | ¹H NMR (400 MHz,MeOH-d₄): δ 2.88 (t, 2H,J = 7.1); 3.10 (t, 2H, J = 6.8);3.95 (s, 3H); 3.97 (s, 3H);6.43 (t, 2H, J = 7.6); 6.74 (d,1H, J = 7.1); 7.16 (s, 1H);7.32 (m, 3H); 7.93 (s, 1H). | 308 | 3.15 (basic) |
| 85 | 6-[2-(3-pyridin-3-ylphenyl)ethyl]pyridin-2-amine | 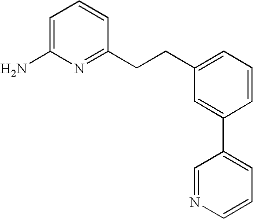 | 16, 19,17 | ¹H NMR (400 MHz,DMSO-d₆): δ 2.84 (t, 2H,J = 7.5); 3.01 (t, 2H, J = 7.5);5.80 (br s, 2H); 6.27 (d, 1H,J = 7.5); 6.38 (d, 1H, J = 7.5);7.24-7.29 (m, 2H); 7.40 (t,1H, J = 7.5); 7.46-7.50 (m,1H); 7.52-7.55 (m, 2H); 8.03-8.06 (m, 1H); 8.57 (d, 1H,J = 5); 8.86 (s, 1H). | 275 | 2.68 (basic) |
| 86 | 6-{2-[3-(5-methoxypyridin-3-yl)phenyl]ethyl}pyridin-2-amine | 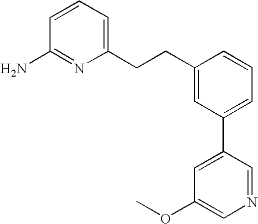 | 16, 17,18, 19 | ¹H NMR (400 MHz,DMSO-d₆): δ 2.84 (t, 2H,J = 7); 3.01 (t, 2H, J = 7);3.92 (s, 3H); 5.80 (br s, 2H);6.27 (d, 1H, J = 8.5); 6.38 (d,1H, J = 7); 7.25-7.29 (m, 2H);7.40 (t, 1H, J = 7.5); 7.53-7.58 (m, 3H); 8.28 (d, 1H,J = 3); 8.45 (d, 1H, J = 2). | 305 | 2.78 (basic) |
| 87 | 6-[2-(3′-methoxy-1,1′-biphenyl-3-yl)ethyl]pyridin-2-2-amine | 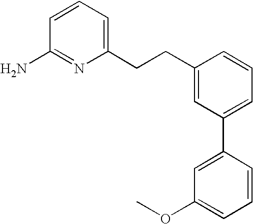 | 16, 17,18, 19 | ¹H NMR (400 MHz,DMSO-d₆): 2.83 (t, 2H,J = 7); 3.00 (t, 2H, J = 7);3.83 (s, 3H); 5.80 (br s, 2H);6.27 (d, 1H, J = 8); 6.37 (d,1H, J = 7); 6.93 (dm, 1H,J = 8); 7.14 (t, 1H, J = 1.5);7.18-7.30 (m, 3H); 7.33-7.39(m, 2H); 7.47 (m, 2H). | 304 | 2.23 (acidic) |
| 88 | 6-{2-[3-(2-furyl)phenyl]ethyl}pyridin-2-amine | 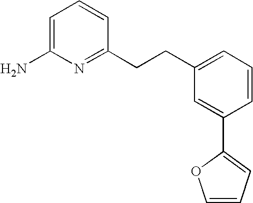 | 16, 17,18, 19 | ¹H NMR (400 MHz,DMSO-d₆): δ 2.80 (m, 2H);2.96 (m, 2H); 5.80 (br s, 2H),6.26 (d, 1H, J = 7.5); 6.37 (d,1H, J = 7.5); 6.59 (dd, 1H,J = 3.5, 1.5); 6.92 (dd, 1H,J = 3.5, 1); 7.14 (d, 1H,J = 7.5); 7.26 (t, 1H, J = 7.5);7.32 (t, 1H, J = 7.5); 7.51 (d,1H, J = 7.5); 7.56 (s, 1H);7.74 (dd, 1H, J = 1.5, 1). | 264 | 2.12 (acidic) |
| 89 | 6-[2-(3′,4′-dimethoxy-1,1′-biphenyl-3-yl)ethyl]pyridin-2-amine | 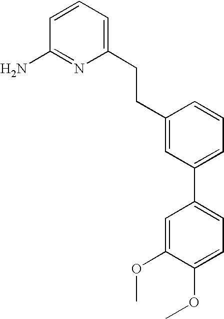 | 16, 17,18, 19 | ¹H NMR (400 MHz,DMSO-d₆): δ 2.82 (m, 2H);2.99 (m, 2H); 3.79 (s, 3H);3.85 (s, 3H); 5.80 (br s, 2H);6.27 (d, 1H, J = 8); 6.37 (d,1H, J = 8); 7.02 (d, 1H,J = 8); 7.14-7.17 (m, 3H);7.26 (t, 1H, J = 7); 7.32 (t,1H, J = 8); 7.42-7.44 (m, 2H). | 334 | 2.16 (acidic) |
| 90 | 6-{2-[3-(1H-indol-6-yl)phenyl]ethyl}pyridin-2-amine | 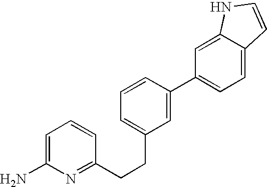 | 16, 18,19 | ¹H NMR (400 MHz,MeOH-d₄): δ 2.86-2.90 (m,2H); 2.93-2.98 (m, 2H); 6.35(d, 1H, J = 3); 6.50 (m, 2H);7.03 (d, 1H, J = 7.5); 7.14-7.16 (m, 2H); 7.22 (t, 1H,J = 7.5); 7.37 (d, 2H, J = 7.5);7.43 (t, 1H, J = 7.5); 7.48 (d,2H, J = 8.5); 8.30 (br s, 1H). | 313 | 2.38 (acidic) |
| 91 | 2-[2-(6-aminopyridin-2-yl)ethyl]-4-(1H-indol-6-yl)phenol | 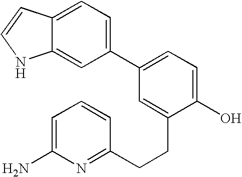 | 16, 18,19 | ¹H NMR (400 MHz,DMSO-d₆): δ 2.86 (t, 2H,J = 6.5); 2.98 (t, 2H, J = 6.5);5.84 (br s, 2H); 6.33 (d, 1H,J = 7.5); 6.46 (m, 2H); 6.92(d, 1H, J = 8.5); 7.25 (dd, 1H,J = 8, 1.5); 7.33-7.39 (m,4H); 7.55 (s, 1H); 7.63 (d,1H, J = 8); 9.45 (s, 1H);11.08 (br s, 1H). | 329 | 2.20 (acidic) |
| 92 | 6-{2-[5-(1H-indol-6-yl)-2-(pyridin-2-ylmethoxy)phenyl]ethyl}pyridin-2-amine | 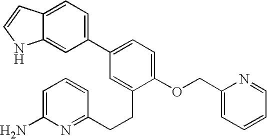 | 16, 18,19 | ¹H NMR (400 MHz,DMSO-d₆): δ 2.92 (t, 2H,J = 7); 3.12 (t, 2H, J = 7);5.32 (s, 2H); 5.83 (br s, 2H);6.33 (d, 1H, J = 7.5); 6.46 (d,1H, J = 7.5); 6.48 (d, 1H,J = 2); 7.13 (d, 1H, J = 8);7.25-7.35 (m, 2H); 7.39-7.43(m, 2H); 7.48-7.53 (m, 2H);7.53-7.63 (m, 2H); 7.67 (d,1H, J = 5); 7.93 (td, 1H,J = 7.5, 1.5); 8.66 (m, 1H);11.13 (br s, 1H). | 420 | 2.26 (acidic) |
| 93 | 6-[2-(3-chloro-1H-indol-6-yl)ethyl]pyridin-2-amine | 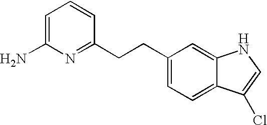 | 20 | ¹H NMR (400 MHz,DMSO-d₆): δ 2.79 (t, 2H,J = 7); 2.98 (t, 2H, J = 7);5.82 (br s, 2H); 6.26 (d, 1H,J = 8); 6.34 (d, 1H, J = 8);7.00 (dd, 1H, J = 8, 1.5); 7.20(s, 1H); 7.23 (t, 1H, J = 8);7.37 (d, 1H, J = 8); 7.42 (d,1H, J = 1.5); 11.20 (br s, 1H). | 271 | 2.16 (acidic) |

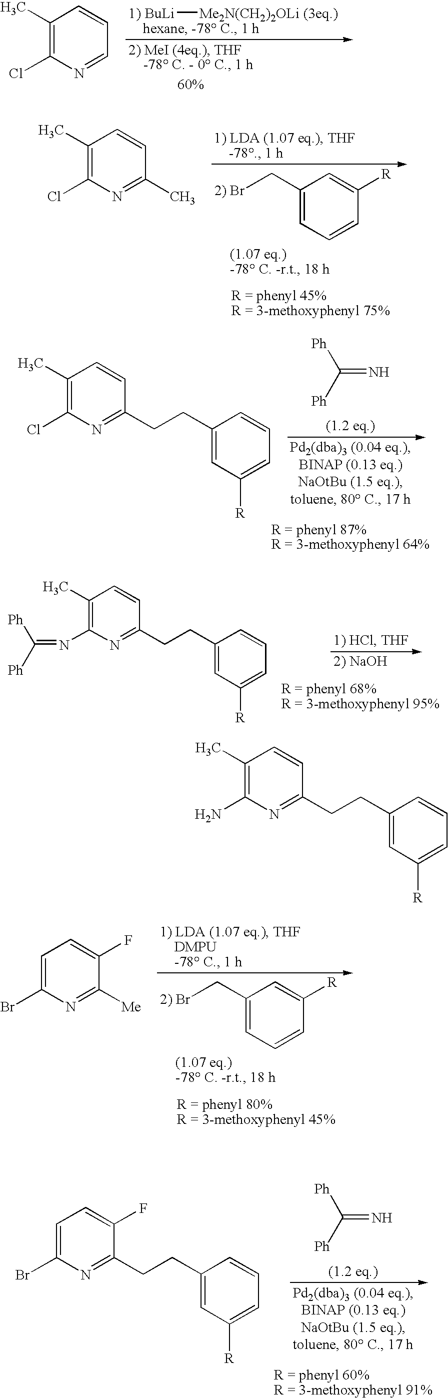

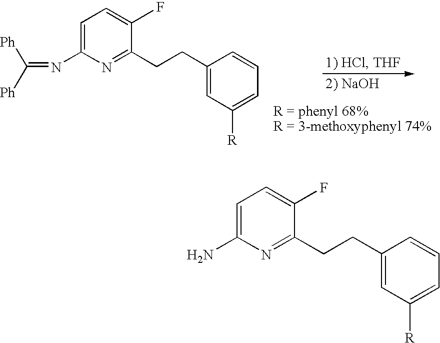

### 6-(2-Biphenyl-3-ylethyl)-2-chloro-3-methylpyridine

General Procedure: Alkylation (Method 21)

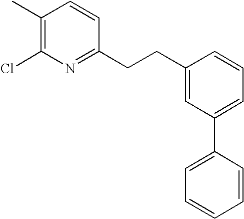

N-Butyllithium (1.5M, 0.47 mL, 0.71 mmol) was added dropwise to a −30° C. stirring solution of diisopropylamine (0.10 mL, 0.71 mmol) in THF (0.35 mL). After 30 minutes, the solution was cooled to −78° C. 2-Chloro-3,6-dimethylpyridine (0.094 g, 0.66 mmol) in THF (0.30 mL) was slowly added. The mixture stirred at −78° C. for 1 hour and then was treated with a solution of 3-(bromomethyl)biphenyl (0.176 g, 0.71 mmol) in THF (0.40 mL). The mixture was allowed to gradually warm to ambient temperature overnight by evaporation of the bath. The solvent was removed in vacuo and the residue partitioned between water and EtOAc. The organic portion was washed (water, brine), dried (MgSO₄), and evaporated to a crude oil that was chromatographed with 19:1 and 9:1 hexane/EtOAc, respectively, to give the product as a colorless oil (0.092 g, 45%). ¹H NMR (300 MHz, CDCl₃): δ 2.35 (s, 3H); 3.09 (s, 4H); 6.92 (d, 1H, J=7.5); 7.13-7.21 (m, 1H), 7.30-7.60 (m, 9H). m/z (APCI) 308 [M+1].

### EXAMPLE 94

### 6-(2-Biphenyl-3-yl-ethyl)-3-methyl-pyridin-2-ylamine

General Procedure: Amination (Method 22)

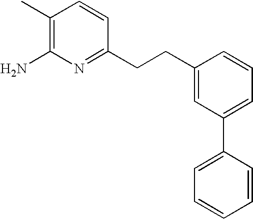

6-(2-Biphenyl-3-ylethyl)-2-chloro-3-methylpyridine (0.090 g, 0.292 mmol), benzophenone imine (0.065 g, 0.358 mmol), sodium t-butoxide (0.042 g, 0.437 mmol), 2,2′-bis(diphenyl-phosphino)-1,1′-binaphthyl (0.024 g, 0.038 mmol), tris(dibenzylideneacetone)dipalladium(0) (0.011 g, 0.012 mmol), and toluene (2.0 mL) were combined under an inert atmosphere. The mixture was stirred in a preheated 80° C. oil bath for 17 hours, cooled, diluted with Et₂O, and filtered through a bed of Celite™. The filtrate was concentrated in vacuo to a crude oil that was chromatographed with 9:1, 3:1, and 1:1 hexane/EtOAc, respectively, to give the product as a yellow oil (0.115 g, 87%). A solution of the oil (0.115 g, 0.254 mmol) in THF (1.7 mL) was treated with 2N HCl (0.086 mL) and stirred for 1 hour. Two additional portions of 2N HCl (0.043 mL) were added at 30 minute intervals and stirring continued until deprotection was complete by TLC. The mixture was concentrated under a stream of N₂ and the residue partitioned between 1N NaOH and DCM. The organic portion was washed (brine), dried (Na₂SO₄), and concentrated to a crude oil that was chromatographed with 2% and 5% 2M ammonia in methanol/DCM, respectively, to give the product as an oil that solidified on standing (0.050g, 68%). ¹H NMR (300 MHz, CDCl₃): δ 2.11 (s, 3H), 2.88-3.12 (m, 4H), 4.37 (br s, 2H), 6.46 (d, 1H, J=7.2), 7.13-7.62 (m, 10H). m/z (APCI) 289 [M+1].

2-Chloro-3,6-dimethylpyridine

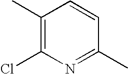

A −5° C. solution of 2-dimethylaminoethanol (2.15 g, 24.1 mmol) in hexane (15.0 mL) was slowly treated with n-butyllithium (1.4M, 35.0 mL, 49.0 mmol) and stirred for 30 minutes. The mixture was cooled to −75° C. and a solution of 2-chloro-3-methylpyridine (1.02 g, 8.0 mmol) in hexane (15.0 mL) was slowly added maintaining the temperature at ≦−70° C. After 1.5 hours, a solution of iodomethane (2.0 mL, 32.1 mmol) in THF (60.0 mL) was added slowly, maintaining the temperature at ≦−70° C. The cold bath was removed and the mixture warmed to 0° C. Water (60.0 mL) was carefully added, the layers separated, and the aqueous portion extracted with diethyl ether. The combined organic portions were washed (water, brine), dried (MgSO₄), and concentrated to a crude oil that was chromatographed with 25% diethyl ether in hexane to give the product as a pale yellow oil (0.674 g, 60%). ¹H NMR (300 MHz, CDCl₃): δ 2.33 (s, 3H); 2.49 (s, 3H); 6.98 (d, 1H, J=7.5); 7.41 (d, 1H, J=7.5).

### (3′-Methoxy-biphenyl-3-yl)-methanol

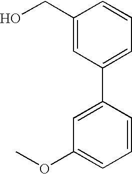

A solution of 3′-methoxy-biphenyl-3-carboxaldehyde (0.385 g, 1.81 mmol) in methanol (8.0 mL) was treated with sodium borohydride (0.072 g, 1.90 mmol) and stirred for 2 hours. The mixture was concentrated in vacuo and the residue was treated with ice water. The aqueous material was saturated with sodium chloride and extracted with Et₂O. The organic portion was washed (brine), dried (MgSO₄), and concentrated to a pale yellow oil (0.387 g, 99%). ¹H NMR (300 MHz, CDCl₃): δ 1.63-1.68 (m, 1H), 3.87 (s, 3H), 4.73-4.80 (m, 2H), 6.87-6.94 (m, 1H), 7.10-7.21 (m, 2H), 7.30-7.62 (m, 5H).

### 3-Bromomethyl-3′-methoxybiphenyl

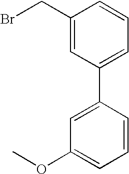

A stirring mixture of (3′methoxy-biphenyl-3-yl)methanol (0.385 g, 1.80 mmol) and 1,2-dibromotetrachloroethane (0.597 g, 1.83 mmol) in THF (5.6 mL) was treated with 1,2-bis(diphenylphosphino)ethane (0.360 g, 0.90 mmol). After 2 hours, the mixture was filtered through a bed of Celite™ and the filtrate concentrated in vacuo. The residue was slurried with 9:1 hexane/EtOAc, placed on a silica gel flash column, and chromatographed with 9:1 and 3:1 hexane/EtOAc, respectively, to give the product as a colorless oil (0.457 g, 91%). ¹HNMR (300 MHz, CDCl₃): δ 3.87 (s,3H), 4.55 (s, 2H), 6.87-6.94 (m, 1H), 7.07-7.20 (m, 2H), 7.32-7.63 (m, 5H).

The following additional examples were similarly prepared by sequential alkylation and amination according to the procedures above: (Methods 21 and 22)

### EXAMPLE 95

### 6-[2-(3′-methoxy-biphenyl-3-yl)-ethyl]-3-methylpyridin-2-ylamine

Synthesized from 2-chloro-3,6-dimethylpyridine and 3-bromomethyl-3′-methoxybiphenyl, with the final product converted to an amine salt with one equivalent of maleic acid after Method 22.

### EXAMPLE 96

### 6-(2-biphenyl-3-ylethyl)-5-fluoro-pyridin-2-ylamine. Synthesized from 2-bromo-5-fluoro-6-methylpyridine and 3-(bromomethyl)biphenyl, with addition of 1,3-dimethyl-3,4,5,6-tetrahydro-2(1H)-pyrimidinone (8 equivalents) to 2-bromo-5-fluoro-6-methyl pyridine in Method 21

### EXAMPLE 97

### 5-fluoro-6-[2-(3 ′-methoxy-biphenyl-3-yl)-ethyl]-pyridin-2-ylamine

Synthesized from 2-bromo-5-fluoro-6-methylpyridine and 3-bromomethyl-3′-methoxybiphenyl, with addition of 1,3-dimethyl-3,4,5,6-tetrahydro-2(1H)-pyrimidinone (44 equivalents) to 2-bromo-5-fluoro-6-methylpyridine in Method 21. The final product was converted to an amine salt with one equivalent of maleic acid after Method 22.

TABLE 9

| | | | | | Molecular | LCMS Rt |
|---|---|---|---|---|---|---|
| Ex. | nme | Structure | Method | NMR | Ion (M+) | (min) |
| 94 | 6-(2-biphenyl-3-ylethyl)-3-methylpyridin-2-ylamine | 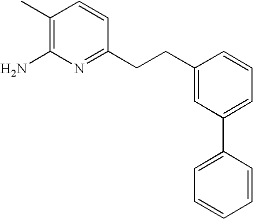 | 21, 22 | ¹H NMR (300 MHz, CDCl₃):δ 2.11 (s, 3H), 2.88-3.12 (m,4H), 4.37 (br s, 2H), 6.46 (d,1H, J = 7.2), 7.13-7.62 (m,10H) | 289 | 1.95(acidic) |
| 95 | 6-[2-(3′-methoxy-biphenyl-3-yl)-ethyl]-3-methylpyridin-2-ylamine | 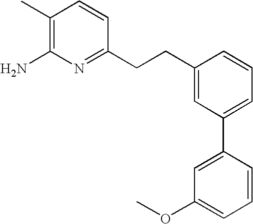 | 21, 22 | ¹H NMR (300 MHz, CDCl₃):δ 2.11 (s, 3H); 2.89-3.12 (m,4H); 3.86 (s, 3H); 4.38 (br s,2H); 6.45 (d, 1H, J = 7.2),6.84-6.92 (m, 1H), 7.06-7.45(m, 8H) | 319 | 2.09(acidic) |
| 96 | 6-(2-biphenyl-3-ylethyl)-5-fluoro-pyridin-2-ylamine | 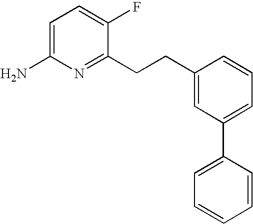 | 21, 22 | ¹H NMR (300 MHz, CDCl₃):δ 2.96-3.12 (m, 4H), 4.20-4.40 (br s, 2H), 6.28-6.36 (m,1H), 7.07-7.64 (m, 10H) | 293 | 1.99(acidic) |
| 97 | 5-fluoro-6-[2-(3′-methoxy-biphenyl-3-yl)-ethyl]-pyridin-2-ylamine | 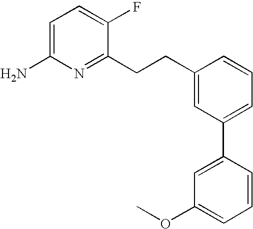 | 21, 22 | ¹H NMR (300 MHz, CDCl₃):δ 2.96-3.12 (m, 4H), 3.87 (s,3H), 4.30 (br s, 2H), 6.27-6.35 (m, 1H), 6.85-6.93 (m,1H), 7.07-7.49 (m, 8H) | 323 | 2.13(acidic) |

1. A compound of formula I: 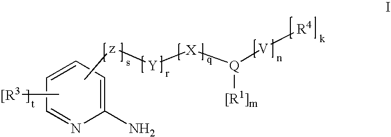
or a pharmaceutically acceptable salt thereof wherein:
Q is selected from C₃₋₁₂cycloalkyl, C₃₋₁₂cycloalkenyl, C₅₋₁₄aryl and C₅₋₁₄heterocyclyl;
R¹ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂;
R^{a} is selected from H, halogen, CN, NH₂, OH, C₁₋₆alkyl, OC₁₋₆alkyl, OC₁₋₆alkyl-OC₁₋₆alkyl, C(═O)C₁₋₆alkyl, C(═O)OC₁₋₆alkyl, OC(═O)C₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂, NHC₃₋₁₂cycloalkyl, N(C₃₋₁₂cycloalkyl)₂, NHC(═O)C₃₋₁₂cycloalkyl, NC(═O)(C₃₋₁₂cycloalkyl)₂, C₅₋₆aryl, OC₅₋₆aryl, C(═O)C₅₋₆aryl, C(═O)OC₅₋₆aryl, C(═O)NH₂, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆)₂, NHC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NH₂, C(═O)NHC₅₋₆heterocyclyl-, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NHC(═O)C₅₋₆heterocyclyl, and NC(═O)(C₅₋₆heterocyclyl)₂;
V is independently selected from N, O, S, SO, SO₂, NHSO₂, SO₂NH, NHC(═O), C(═O)NH, and optionally substituted C₁₋₆alkyl wherein such substituent is/are independently selected from R²;
X is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;
Y is selected from N, O, S, SO, SO₂, NHSO₂, SO₂NH, NHC(═O), C(═O)NH, and optionally substituted C₁₋₆alkyl or C₃₋₅cycloalkyl wherein such substituent is/are independently selected from R²;
Z is selected from N, O, S, SO, SO₂, NHSO₂, SO₂NH, NHC(═O), C(═O)NH, and optionally substituted C₁₋₆alkyl or C₃₋₅cycloalkyl wherein such substituent is/are independently selected from R²;
k is 0, 1, 2, 3 or 4;
m is 0, 1, 2, 3 or 4;
n is 0, 1, 2, 3 or 4;
q is 0 or 1;
r is 0 or 1;
s is 0 or 1;
t is 0, 1 or 2;
where at least one of s, r or q are 1.
R² is independently selected from H, halogen, optionally substituted C₁₋₆alkyl, optionally substituted O—C₁₋₆alkyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, and NC(═O)(C₅₋₆heterocyclyl)₂;
R³ is independently selected from H, halogen, CN, NH₂, OH, C₁₋₆alkyl-R^{a}, C₂₋₆alkenyl-R^{a}, C₂₋₆alkynyl-R^{a}, C₅₋₆cycloalkenyl-R^{a}, C₃₋₁₂cycloalkyl-R^{a}, C₁₋₆alkyl-C₃₋₁₂cycloalkyl-R^{a}, C₅₋₁₀aryl-Rₐ, C₁₋₆alkyl-C₅₋₁₀aryl-R^{a}, C₅₋₁₀heterocyclyl-R^{a}, C₁₋₆alkyl-C₅₋₁₀heterocyclyl-R^{a}, C(═O)C₅₋₆heterocyclyl-R^{a}, C(═O)C₅₋₁₀aryl-R^{a}, C(═O)C₃₋₁₂cycloalkyl-R^{a}, OC₁₋₆alkyl-R^{a}, O—C₅₋₁₀aryl-R^{a}, O—C₅₋₆heterocyclyl-R^{a}, O—C₃₋₁₂cycloalkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl)-R^{a}, SO₂NHC₁₋₆alkyl-R^{a}, SO₂N(C₁₋₆alkyl)₂-R^{a}, NH(C₁₋₆alkyl)-R^{a}, R^{a}, N(C₁₋₆alkyl)₂-R^{a}, NHC(═O)C₁₋₆alkyl, NC(═O)(C₁₋₆alkyl)₂, NHC(═O)NHC₁₋₆alkyl, NHC(═O)OC₁₋₆alkyl, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHNH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, NHC(═O)NHC₅₋₆aryl, NHC(═O)OC₅₋₆aryl, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂, NHC(═O)NHC₅₋₆heterocyclyl, and NHC(═O)OC₅₋₆heterocyclyl;
R⁴ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂.

2. A compound of formula Ia: 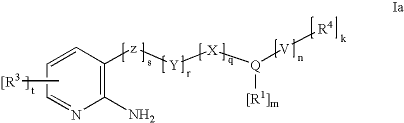
or a pharmaceutically acceptable salt thereof wherein:
Q is selected from C₃₋₁₂cycloalkyl, C₃₋₁₂cycloalkenyl, C₅₋₁₄aryl and C₅₋₁₄heterocyclyl;
R¹ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂;
R^{a} is selected from H, halogen, CN, NH₂, OH, C₁₋₆alkyl, OC₁₋₆alkyl, OC₁₋₆alkyl-OC₁₋₆alkyl, C(═O)C₁₋₆alkyl, C(═O)OC₁₋₆alkyl, OC(═O)C₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂,_NHC₃₋₁₂cycloalkyl, N(C₃₋₁₂cycloalkyl)₂, NHC(═O)C₃₋₁₂cycloalkyl, NC(═O)(C₃₋₁₂cycloalkyl)₂, C₅₋₆aryl, OC₅₋₆aryl, C(═O)C₅₋₆aryl, C(═O)OC₅₋₆aryl, C(═O)NH₂, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NHC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NH₂, C(═O)NHC₅₋₆heterocyclyl-, C(═O)N(C₅₋₆heterocyclyl)₂, SO₂C₅₋₆heterocyclyl, SO₂NHC₅₋₆heterocyclyl, SO₂N(C₅₋₆heterocyclyl)₂NH(C₅₋₆heterocyclyl), N(C₅₋₆heterocyclyl)₂, NHC(═O)C₅₋₆heterocyclyl, and NC(═O)(C₅₋₆heterocyclyl)₂;
V is independently selected from N, O, S, SO, SO₂, NHSO₂, SO₂NH, NHC(═O), C(═O)NH, and optionally substituted C₁₋₆alkyl wherein such substituent is/are independently selected from R²;
X is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;
Y is selected from N, O, S, SO, SO₂, NHSO₂, SO₂NH, NHC(═O), C(═O)NH, and optionally substituted C₁₋₆alkyl or C₃₋₅cycloalkyl wherein such substituent is/are independently selected from R²;
Z is selected from N, O, S, SO, SO₂, NHSO₂, SO₂NH, NHC(═O), C(═O)NH, and optionally substituted C₁₋₆alkyl or C₃₋₅cycloalkyl wherein such substituent is/are independently selected from R²;
k is 0, 1, 2, 3 or 4;
m is 0, 1, 2, 3 or 4;
n is 0, 1, 2, 3 or 4;
q is 0 or 1;
r is 0 or 1;
s is 0 or 1;
t is 0, 1 or 2;
where at least one of s, r or q are 1.
R² is independently selected from H, halogen, optionally substituted C₁₋₆alkyl, optionally substituted O—C₁₋₆alkyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C_{5 6}aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, and NC(═O)(C₅₋₆heterocyclyl)₂;
R³ is independently selected from H, halogen, CN, NH₂, OH, C₁₋₆alkyl-R^{a}, C₂₋₆alkenyl-R^{a}, C₂₋₆alkynyl-R^{a}, C₅₋₆cycloalkenyl-R^{a}, C₃₋₁₂cycloalkyl-R^{a}, C₁₋₆alkyl-C₃₋₁₂cycloalkyl-R^{a}, C₅₋₁₀aryl-R^{a}, C₁₋₆alkyl-C₅₋₁₀aryl-R^{a}, C₅₋₁₀heterocyclyl-R^{a}, C₁₋₆alkyl-C₅₋₁₀heterocyclyl-R^{a}, C(═O)C₅₋₆heterocyclyl-R^{a}, C(═O)C₅₋₁₀aryl-R^{a}, C(═O)C₃₋₁₂cycloalkyl-R^{a}, OC₁₋₆alkyl-R^{a}, O—C₅₋₁₀aryl-R^{a}, O—C₅₋₆heterocyclyl-R^{a}, O—C₃₋₁₂cycloalkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, NHC(═O)NHC₁₋₆alkyl, NHC(═O)OC₁₋₆alkyl, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHNH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, NH(C₅₋₆aryl)-R^{a}, N(C₅₋₆aryl)₂-R^{a}, NHC(═O)C₅₋₆aryl, NC(═O)(C₅₋₆aryl)₂, NHC(═O)NHC₅₋₆aryl, NHC(═O)OC₅₋₆aryl, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂, NHC(═O)NHC₅₋₆heterocyclyl, and NHC(═O)OC₅₋₆heterocyclyl;
R⁴ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{, OC}₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂.

3. A compound of formula Ib: 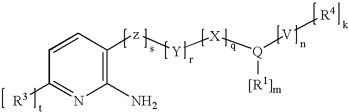
or a pharmaceutically acceptable salt thereof wherein:
Q is selected from C₃₋₁₂cycloalkyl, C₃₋₁₂cycloalkenyl, C₅₋₁₄aryl and C₅₋₁₄heterocyclyl;
R¹ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC-₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆hetercyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-Rₐ)₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂;
R^{a} is selected from H, halogen, CN, NH₂, OH, C₁₋₆alkyl, OC₁₋₆alkyl, OC₁₋₆alkyl-OC₁₋₆alkyl, C(═O)C₁₋₆alkyl, C(═O)OC₁₋₆alkyl, OC(═O)C₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂, NHC₃₋₁₂cycloalkyl, N(C₃₋₁₂cycloalkyl)₂, NHC(═O)C₃₋₁₂cycloalkyl, NC(═O)(C₃₋₁₂cycloalkyl)₂, C₅₋₆aryl, OC₅₋₆aryl, C(═O)C₅₋₆aryl, C(═O)OC₅₋₆aryl, C(═O)NH₂, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl, N(C₅₋₆aryl)₂, NHC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NH₂, C(═O)NHC₅₋₆heterocyclyl-, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NHC(═O)C₅₋₆heterocyclyl, and NC(═O)(C₅₋₆heterocyclyl)₂;
V is independently selected from N, O, S, SO, SO₂, NHSO₂, SO₂NH, NHC(═O), C(═O)NH, and optionally substituted C₁₋₆alkyl wherein such substituent is/are independently selected from R²;
X is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;
Y is selected from N, O, S, SO, SO₂, NHSO₂, SO₂NH, NHC(═O), C(═O)NH, and optionally substituted C₁₋₆alkyl or C₃₋₅cycloalkyl wherein such substituent is/are independently selected from R²;
Z is selected from N, O, S, SO, SO₂, NHSO₂, SO₂NH, NHC(═O), C(═O)NH, and optionally substituted C₁₋₆alkyl or C₃₋₅cycloalkyl wherein such substituent is/are independently selected from R²;
k is 0, 1, 2, 3 or 4;
m is 0, 1, 2, 3 or 4;
n is 0, 1, 2, 3 or 4;
q is 0 or 1;
r is 0 or 1;
s is 0 or 1;
t is 0, or 1;
where at least one of s, r or q are 1.
R² is independently selected from H, halogen, optionally substituted C₁₋₆alkyl, optionally substituted O—C₁₋₆alkyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, and NC(═O)(C₅₋₆heterocyclyl)₂;
R³ is independently selected from H, halogen, CN, NH₂, OH, C₁₋₆alkyl-R^{a}, C₂₋₆alkenyl-R^{a}, C₂₋₆alkynyl-R^{a}, C₅₋₆cycloalkenyl-R^{a}, C₃₋₁₂cycloalkyl-R^{a}, C₁₋₆alkyl-C₃₋₁₂cycloalkyl-R^{a}, C₅₋₁₀aryl-R^{a}, C₁₋₆alkyl-C₅₋₁₀aryl-R^{a}, C₅₋₁₀heterocyclyl-R^{a}, C₁₋₆alkyl-C₅₋₁₀heterocyclyl-R^{a}, C(═O)C₅₋₆heterocyclyl-R^{a}, C(═O)C₅₋₁₀aryl-R^{a}, C(═O)C₃₋₁₂cycloalkyl-R^{a}, OC₁₋₆alkyl-R^{a}, O—C₅₋₁₀aryl-R^{a}, O—C₅₋₆heterocyclyl-R^{a}, O—C₃₋₁₂cycloalkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, NHC(═O)NHC₁₋₆alkyl, NHC(═O)OC₁₋₆alkyl, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHNH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, NHC(═O)NHC₅₋₆aryl, NHC(═O)OC₅₋₆aryl, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂, NHC(═O)NHC₅₋₆heterocyclyl, and NHC(═O)OC₅₋₆heterocyclyl;
R⁴ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₆heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₁₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂.

4. A compound of formula Ic: 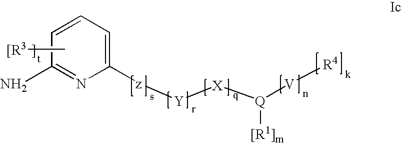
or a pharmaceutically acceptable salt thereof wherein:
Q is selected from C₃₋₁₂cycloalkyl, C₃₋₁₂cycloalkenyl, C₅₋₁₄aryl and C₅₋₁₄heterocyclyl;
R¹ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂;
R^{a} is selected from H, halogen, CN, NH₂, OH, C₁₋₆alkyl, OC₁₋₆alkyl, OC₁₋₆alkyl-OC₁₋₆alkyl, C(═O)C₁₋₆alkyl, C(═O)OC₁₋₆alkyl, OC(═O)C₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂, NHC₃₋₁₂cycloalkyl, N(C₃₋₁₂cycloalkyl)₂, NHC(═O)C₃₋₁₂cycloalkyl, NC(═O)(C₃₋₁₂cycloalkyl)₂, C₅₋₆aryl, OC₅₋₆aryl, C(═O)C₅₋₆aryl, C(═O)OC₅₋₆aryl, C(═O)NH₂, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NHC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NH₂, C(═O)NHC₅₋₆heterocyclyl-, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NHC(═O)C₅₋₆heterocyclyl, and NC(═O)(C₅₋₆heterocyclyl)₂;
V is independently selected from N, O, S, SO, SO₂, NHSO₂, SO₂NH, NHC(═O), C(═O)NH, and optionally substituted C₁₋₆alkyl wherein such substituent is/are independently selected from R²;
X is selected from N, O, S, SO, SO₂, NHSO₂, SO₂NH, NHC(═O), C(═O)NH, and optionally substituted C₁₋₆alkyl, C₂₋₆alkenyl or C₃₋₅cycloalkyl wherein such substituent is/are independently selected from R²;
Y is selected from N, O, S, SO, SO₂, NHSO₂, SO₂NH, NHC(═O), C(═O)NH, and optionally substituted C₁₋₆alkyl or C₃₋₅cycloalkyl wherein such substituent is/are independently selected from R²;
Z is selected from N, O, S, SO, SO₂, NHSO₂, SO₂NH, NHC(═O), C(═O)NH, and optionally substituted C₁₋₆alkyl or C₃₋₅cycloalkyl wherein such substituent is/are independently selected from R²;
k is 0, 1, 2, 3 or 4;
m is 0, 1, 2, 3 or 4;
n is 0, 1, 2, 3 or 4;
q is 0 or 1;
r is 0 or 1;
s is 0 or 1;
t is 0, 1 or 2;
where at least one of s, r or q are 1.
R² is independently selected from H, halogen, optionally substituted C₁₋₆alkyl, optionally substituted O—C₁₋₆alkyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituent are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alkyl-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, and NC(═O)(C₅₋₆heterocyclyl)₂;
R³ is independently selected from H, halogen, CN, NH₂, OH, C₁₋₆alkyl-R^{a}, C₂₋₆alkenyl-R^{a}, C₂₋₆alkynyl-R^{a}, C₅₋₆cycloalkenyl-R^{a}, C₃₋₁₂cycloalkyl-R^{a}, C₁₋₆alkyl-C₃₋₁₂cycloalkyl-R^{a}, C₅₋₁₀aryl-R^{a}, C₁₋₆alkyl-C₅₋₁₀aryl-R^{a}, C₅₋₁₀heterocyclyl-R^{a}, C₁₋₆alkyl-C₅₋₁₀heterocyclyl-R^{a}, C(═O)C₅₋₆heterocyclyl-R^{a}, C(═O)C₅₋₁₀aryl-R^{a}, C(═O)C₃₋₁₂cycloalkyl-R^{a}, OC₁₋₆alkyl-R^{a}, O—C₅₋₁₀aryl-R^{a}, O—C₅₋₆heterocyclyl-R^{a}, O—C₃₋₁₂cycloalkyl-R^{a}, C(═O)OC₁₋₆alkyl-R^{a}, C(═O)NH₂, C(═O)NHC₁₋₆alkyl-R^{a}, C(═O)N(C₁₋₆alkyl)₂-R^{a}, SO₂C₁₋₆alky l-R^{a}, SO₂NHC₁₋₆alkyl-R^{a}, SO₂N(C₁₋₆alkyl)₂-R^{a}, NH(C₁₋₆alkyl)-R^{a}, N(C₁₋₆alkyl)₂-R^{a}, NHC(═O)C₁₋₆alkyl, NC(═O)(C₁₋₆alkyl)₂, NHC(═O)NHC₁₋₆alkyl, NHC(═O)OC₁₋₆alkyl, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHNH₂, C(═O)NHC₅₋₆aryl-R^{a}, C(═O)N(C₅₋₆aryl)₂-R^{a}, SO₂C₅₋₆aryl-R^{a}, SO₂NHC₅₋₆aryl-R^{a}, SO₂N(C₅₋₆aryl)₂-R^{a}, NH(C₅₋₆aryl)-R^{a}, N(C₅₋₆aryl)₂-R^{a}, NHC(═O)C₅₋₆aryl, NC(═O)(C₅₋₆aryl)₂, NHC(═O)NHC₅₋₆aryl, NHC(═O)OC₅₋₆aryl, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂, NHC(═O)NHC₅₋₆heterocyclyl, and NHC(═O)OC₅₋₆heterocyclyl;
R⁴ is independently selected from H, OH, halogen, N(C₁₋₄alkyl )₂, NHC₁₋₄alkyl, NH₂, optionally substituted C₁₋₆alkyl, optionally substituted OC₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted C₅₋₁₀heterocyclyl and optionally substituted C₁₋₆alkyl-C₅₋₁₀heterocyclyl wherein such substituents is/are independently selected from: halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl-R^{a})₂, SO₂C₁₋₆alkyl-R^{a}, SO₂NHC₁₋₆alkyl-R^{a}, SO₂N(C₁₋₆alkyl-R^{a})₂, NH(C₁₋₆alkyl)-R^{a}, N(C₁₋₆alkyl-R^{a})₂, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl-R^{a})₂, SO₂C₅₋₆aryl-R^{a}, SO₂NHC₅₋₆aryl-R^{a}, SO₂N(C₅₋₆aryl-R^{a})₂, NH(C₅₋₆aryl)-R^{a}, N(C₅₋₆aryl-R^{a})₂, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl-R^{a})₂, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl-R^{a})₂, NHC(═O)C₅₋₆heterocyclyl, and NC(═O)(C₅₋₆heterocyclyl)₂.

5. A compound of formula (I) or a pharmaceutically salt thereof as recited in claim 1 wherein R¹, R^{a}, V, X, Y, Z, m, n, q, r, s, t, R², R³ and R⁴ have the meanings defined in claim 1 and Q is C₅₋₁₀aryl.

6. A compound of formula (Ia) or a pharmaceutically salt thereof as recited in claim 2 wherein R¹, R^{a}, V, X, Y, Z, m, n, q, r, s, t, R², R³ and R⁴ have the meanings defined in claim 2 and Q is C₅₋₁₀aryl.

7. A compound of formula (Ib) or a pharmaceutically salt thereof as recited in claim 3 wherein R¹, R^{a}, V, X, Y, Z, m, n, q, r, s, t, R², R³, and R⁴ have the meanings defined in claim 3 and Q is C₅₋₁₀aryl.

8. A compound of formula (Ic) or a pharmaceutically salt thereof as recited in claim 4 wherein R¹, R^{a}, V, X, Y, Z, m, n, q, r, s, t, R², R³ and R⁴ have the meanings defined in claim 4 and Q is C₅₋₁₀aryl.

9. A compound of formula (I) or a pharmaceutically salt thereof as recited in claim 1 wherein R¹, R^{a}, V, X, Y, Z, m, n, q, r, s, t, R², R³ and R⁴ have the meanings defined in claim 1 and Q is phenyl.

10. A compound of formula (Ia) or a pharmaceutically salt thereof as recited in claim 2 wherein R¹, R^{a}, V, X, Y, Z, m, n, q, r, s, t, R², R³ and R⁴ have the meanings defined in claim 2 and Q is phenyl.

11. A compound of formula (Ib) or a pharmaceutically salt thereof as recited in claim 3 wherein R¹, R^{a}, V, X, Y, Z, m, n, q, r, s, t, R², R³ and R⁴ have the meanings defined in claim 3 and Q is phenyl.

12. A compound of formula (Ic) or a pharmaceutically salt thereof as recited in claim 4 wherein R¹, R^{a}, V, X, Y, Z, m, n, q, r, s, t, R², R³ and R⁴ have the meanings defined in claim 4 and Q is phenyl.

13. A compound of formula (I) or a pharmaceutically salt thereof as recited in claim 1 wherein Q, V, X, Y, Z, m, n, q, r, s, t, R², R³ and R⁴ have the meanings defined in claim 1 and R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R¹ and wherein R^{a} is independently selected from H, CN, OH, and C₁₋₆alkyl.

14. A compound of formula (Ia) or a pharmaceutically salt thereof as recited in claim 2 wherein Q, V, X, Y, Z, m, n, q, r, s, t, R², R³ and R⁴ have the meanings defined in claim 2 and R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆-alkyl-R^{a}, OC₁₋₆alkyl-R^{a}, C(═O)OC₁₋₆alkyl-R^{a}, and C₅₋₆heterocyclyl-R^{a} and wherein R^{a} is independently selected from H, CN, OH, and C₁₋₆alkyl.

15. A compound of formula (Ib) or a pharmaceutically salt thereof as recited in claim 3 wherein Q, V, X, Y, Z, m, n, q, r, s, t, R², R³ and R⁴ have the meanings defined in claim 3 and R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a} and wherein R^{a} is independently selected from H, CN, OH, and C₁₋₆alkyl.

16. A compound of formula (Ic) or a pharmaceutically salt thereof as recited in claim 4 wherein Q, V, X, Y, Z, m, n, q, r, s, t, R², R³ and R⁴ have the meanings defined in claim 4 and R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a} and wherein R^{a} is independently selected from H, CN, OH, and C₁₋₆alkyl.

17. A compound of formula (I) or a pharmaceutically salt thereof as recited in claim 1 wherein Q, R¹, R^{a}, X, Y, Z, m, n, q, r, s, t, R², R³ and R⁴ have the meanings defined in claim 1 and V is O.

18. A compound of formula (Ia) or a pharmaceutically salt thereof as recited in claim 2 wherein Q, R¹, R^{a}, X, Y, Z, m, n, q, r, s, t, R², R³ and R⁴ have the meanings defined in claim 2 and V is O.

19. A compound of formula (Ib) or a pharmaceutically salt thereof as recited in claim 3 wherein Q, R¹, R^{a}, X, Y, Z, m, n, q, r, s, t, R², R³ and R⁴ have the meanings defined in claim 3 and V is O.

20. A compound of formula (Ic) or a pharmaceutically salt thereof as recited in claim 4 wherein Q, R¹, R^{a}, X, Y, Z, m, n, q, r, s, t, R², R³ and R⁴ have the meanings defined in claim 4 and V is O.

21. A compound of formula (I) or a pharmaceutically salt thereof as recited in claim I wherein Q, R¹, R^{a}, V, Y, Z, m, n, q, r, s, t, R², R³ and R⁴ have the meanings defined in claim 1 and X is C₁₋₆alkyl.

22. A compound of formula (Ia) or a pharmaceutically salt thereof as recited in claim 2 wherein Q, R¹, R^{a}, V, Y, Z, m, n, q, r, s, t, R², R³ and R⁴ have the meanings defined in claim 2 and X is C₁₋₆alkyl.

23. A compound of formula (Ib) or a pharmaceutically salt thereof as recited in claim 3 wherein Q, R¹, R^{a}, V, Y, Z, m, n, q, r, s, t, R², R³ and R⁴ have the meanings defined in claim 3 and X is C₁₋₆alkyl.

24. A compound of formula (Ic) or a pharmaceutically salt thereof as recited in claim 4 wherein Q, R¹, R^{a}, V, Y, Z, m, n, q, r, s, t, R², R³ and R⁴ have the meanings defined in claim 4 and X is C₁₋₆alkyl.

25. A compound of formula (I) or a pharmaceutically salt thereof as recited in claim 1 wherein Q, R¹, R^{a}, V, X, Z, m, n, q, r, s, t, R², R³ and R⁴ have the meanings defined in claim 1 and Y is C₁₋₆alkyl.

26. A compound of formula (Ia) or a pharmaceutically salt thereof as recited in claim 2 wherein Q, R¹, R^{a}, V, X, Z, m, n, q, r, s, t, R², R³ and R⁴ have the meanings defined in claim 2 and Y is C₁₋₆alkyl.

27. A compound of formula (Ib) or a pharmaceutically salt thereof as recited in claim 3 wherein Q, R¹, R^{a}, V, X, Z. m, n, q, r, s, t, R², R³ and R⁴ have the meanings defined in claim 3 and Y is C₁₋₆alkyl.

28. A compound of formula (Ic) or a pharmaceutically salt thereof as recited in claim 4 wherein Q, R¹, R^{a}, V, X, Z. m, n, q, r, s, t, R², R³ and R⁴ have the meanings defined in claim 4 and Y is C₁₋₆alkyl.

29. A compound of formula (I) or a pharmaceutically salt thereof as recited in claim 1 wherein Q, R¹, R^{a}, V, X, Y, m, n, q, r, s, t, R², R³ and R⁴ have the meanings defined in claim 1 and Z is N or C₁₋₆alkyl.

30. A compound of formula (Ia) or a pharmaceutically salt thereof as recited in claim 2 wherein Q, R¹, R^{a}, V, X, Y, m, n, q, r, s, t, R², R³ and R⁴ have the meanings defined in claim 2 and Z is N or C₁₋₆alkyl.

31. A compound of formula (Ib) or a pharmaceutically salt thereof as recited in claim 3 wherein Q, R¹, R^{a}, V, X, Y, m, n, q, r, s, t, R², R³ and R⁴ have the meanings defined in claim 3 and Z is N or C₁₋₆alkyl.

32. A compound of formula (Ic) or a pharmaceutically salt thereof as recited in claim 4 wherein Q, R¹, R^{a}, V, X, Y, m, n, q, r, s, t, R², R³ and R⁴ have the meanings defined in claim 4 and Z is N or C₁₋₆alkyl.

33. A compound of formula (I) or a pharmaceutically salt thereof as recited in claim 1 wherein Q, R¹, R^{a}, V, X, Y, Z, n, q, r, s, t, R², R³ and R⁴ have the meanings defined in claim 1 and k is 0, 1, or 2.

34. A compound of formula (Ia) or a pharmaceutically salt thereof as recited in claim 2 wherein Q, R¹, R^{a}, V, X, Y, Z, n, q, r, s, t, R², R³ and R⁴ have the meanings defined in claim 2 and k is 0, 1, or 2.

35. A compound of formula (Ib) or a pharmaceutically salt thereof as recited in claim 3 wherein Q, R¹, R^{a}, V, X, Y, Z, n, q, r, s, t, R², R³ and R⁴ have the meanings defined in claim 3 and k is 0, 1, or 2.

36. A compound of formula (Ic) or a pharmaceutically salt thereof as recited in claim 4 wherein Q, R¹, R^{a}, V, X, Y, Z, n, q, r, s, t, R², R³ and R⁴ have the meanings defined in claim 4 and k is 0, 1, or 2.

37. A compound of formula (I) or a pharmaceutically salt thereof as recited in claim 1 wherein Q, R¹, R^{a}, V, X, Y, Z, n, q, r, s, t, R², R³ and R⁴ have the meanings defined in claim 1 and m is 0, 1, or 2.

38. A compound of formula (Ia) or a pharmaceutically salt thereof as recited in claim 2 wherein Q, R¹, R^{a}, V, X, Y, Z, n, q, r, s, t, R², R³ and R⁴ have the meanings defined in claim 2 and m is 0, 1, or 2.

39. A compound of formula (Ib) or a pharmaceutically salt thereof as recited in claim 3 wherein Q, R¹, R^{a}, V, X, Y, Z, n, q, r, s, t, R², R³ and R⁴ have the meanings defined in claim 3 and m is 0, 1, or 2.

40. A compound of formula (Ic) or a pharmaceutically salt thereof as recited in claim 3 wherein Q, R¹, R^{a}, V, X, Y, Z, n, q, r, s, t, R², R³ and R⁴ have the meanings defined in claim 3 and m is 0, 1, or 2.

41. A compound of formula (I) or a pharmaceutically salt thereof as recited in claim 1 wherein Q, R¹, R^{a}, V, X, Y, Z, m, q, r, s, t, R², R³ and R⁴ have the meanings defined in claim 1 and n is 0, 1, or 2.

42. A compound of formula (Ia) or a pharmaceutically salt thereof as recited in claim 2 wherein Q, R¹, R^{a}, V, X, Y, Z, m, q, r, s, t, R², R³ and R⁴ have the meanings defined in claim 2 and n is 0, 1, or 2.

43. A compound of formula (Ib) or a pharmaceutically salt thereof as recited in claim 3 wherein Q, R¹, R^{a}, V, X, Y, Z, m, q, r, s, t, R², R³ and R⁴ have the meanings defined in claim 3 and n is 0, 1, or 2.

44. A compound of formula (Ic) or a pharmaceutically salt thereof as recited in claim 4 wherein Q, R¹, R^{a}, V, X, Y, Z, m, q, r, s, t, R², R³ and R⁴ have the meanings defined in claim 4 and n is 0, 1, or 2.

45. A compound of formula (I) or a pharmaceutically salt thereof as recited in claim 1 wherein Q, R¹, R^{a}, V, X, Y, Z, m, n, r, s, t, R², R³ and R⁴ have the meanings defined in claim 1 and q is 0.

46. A compound of formula (Ia) or a pharmaceutically salt thereof as recited in claim 2 wherein Q, R¹, R^{a}, V, X, Y, Z, m, n, r, s, t, R², R³ and R⁴ have the meanings defined in claim 2 and q is 0.

47. A compound of formula (Ib) or a pharmaceutically salt thereof as recited in claim 3 wherein Q, R¹, R^{a}, V, X, Y, Z, m, n, r, s, t, R², R³ and R⁴ have the meanings defined in claim 3 and q is 0.

48. A compound of formula (Ic) or a pharmaceutically salt thereof as recited in claim 4 wherein Q, R¹, R^{a}, V, X, Y, Z, m, n, r, s, t, R², R³ and R⁴ have the meanings defined in claim 4 and q is 0.

49. A compound of formula (I) or a pharmaceutically salt thereof as recited in claim 1 wherein Q, R¹, R^{a}, V, X, Y, Z, m, n, q, s, t, R², R³ and R⁴ have the meanings defined in claim 1 and r is 1.

50. A compound of formula (Ia) or a pharmaceutically salt thereof as recited in claim 2 wherein Q, R¹, R^{a}, V, X, Y, Z, m, n, q, s, t, R², R³ and R⁴ have the meanings defined in claim 2 and r is 1.

51. A compound of formula (Ib) or a pharmaceutically salt thereof as recited in claim 3 wherein Q, R¹, R^{a}, V, X, Y, Z, m, n, q, s, t, R², R³ and R⁴ have the meanings defined in claim 3 and r is 1.

52. A compound of formula (Ic) or a pharmaceutically salt thereof as recited in claim 4 wherein Q, R¹, R^{a}, V, X, Y, Z, m, n, q, s, t, R², R³ and R⁴ have the meanings defined in claim 4 and r is 1.

53. A compound of formula (I) or a pharmaceutically salt thereof as recited in claim 1 wherein Q, R¹, R^{a}, V, X, Y, Z, m, n, q, t, R², R³ and R⁴ have the meanings defined in claim 1 and s is 1.

54. A compound of formula (Ia) or a pharmaceutically salt thereof as recited in claim 2 wherein Q, R¹, R^{a}, V, X, Y, Z, m, n, q, t, R², R³ and R⁴ have the meanings defined in claim 2 and s is 1.

55. A compound of formula (Ib) or a pharmaceutically salt thereof as recited in claim 3 wherein Q, R¹, R^{a}, V, X, Y, Z, m, n, q, t, R², R³ and R⁴ have the meanings defined in claim 3 and s is 1.

56. A compound of formula (Ic) or a pharmaceutically salt thereof as recited in claim 4 wherein Q, R¹, R^{a}, V, X, Y, Z, m, n, q, t, R², R³ and R⁴ have the meanings defined in claim 4 and s is 1.

57. A compound of formula (I) or a pharmaceutically salt thereof as recited in claim 1 wherein Q, R¹, R^{a}, V, X, Y, Z, m, n, q, r, s, t, R³ and R⁴ have the meanings defined in claim 1 and R² is independently selected from H, or C₁₋₆alkyl.

58. A compound of formula (Ia) or a pharmaceutically salt thereof as recited in claim 2 wherein Q, R¹, R^{a}, V, X, Y, Z. m, n, q, r, s, t, R³ and R⁴ have the meanings defined in claim 2 and R² is independently selected from H, or C₁₋₆alkyl.

59. A compound of formula (Ib) or a pharmaceutically salt thereof as recited in claim 3 wherein Q, R¹, R^{a}, V, X, Y, Z, m, n, q, r, s, t, R³ and R⁴ have the meanings defined in claim 3 and R² is independently selected from H, or C₁₋₆alkyl.

60. A compound of formula (Ic) or a pharmaceutically salt thereof as recited in claim 4 wherein Q, R¹, R^{a}, V, X, Y, Z, m, n, q, r, s, t, R³ and R⁴ have the meanings defined in claim 4 and R² is independently selected from H, or C₁₋₆alkyl.

61. A compound of formula (I) or a pharmaceutically salt thereof as recited in claim 1 wherein Q, R¹, R^{a}, V, X, Y, Z, m, n, q, r, s, t, R² and R⁴ have the meanings defined in claim 1 and R³is independently selected from H, halogen, C₁₋₆alkyl-R^{a} wherein R^{a} is independently selected from H, CN, OH, or C₁₋₆alkyl.

62. A compound of formula (Ia) or a pharmaceutically salt thereof as recited in claim 2 wherein Q, R¹, R^{a}, V, X, Y, Z, m, n, q, r, s, t, R² and R⁴ have the meanings defined in claim 2 and R³ is independently selected from H, halogen, C₁₋₆alkyl-R^{a} wherein R^{a} is independently selected from H, CN, OH, or C₁₋₆alkyl.

63. A compound of formula (Ib) or a pharmaceutically salt thereof as recited in claim 3 wherein Q, R¹, R^{a}, V, X, Y, Z, m, n, q, r, s, t, R² and R⁴ have the meanings defined in claim 3 and R³ is independently selected from H, halogen, C₁₋₆alkyl-R^{a} wherein R^{a} is independently selected from H, CN, OH, or C₁₋₆alkyl.

64. A compound of formula (Ic) or a pharmaceutically salt thereof as recited in claim 4 wherein Q, R¹, R^{a}, V, X, Y, Z, m, n, q, r, s, t, R² and R⁴ have the meanings defined in claim 4 and R³ is independently selected from H, halogen, C₁₋₆alkyl-R^{a} wherein R^{a} is independently selected from H, CN, OH, or C₁₋₆alkyl.

65. A compound of formula (I) or a pharmaceutically salt thereof as recited in claim 1 wherein Q, V, X, Y, Z, m, n, q, r, s, t, R² and R³ have the meanings defined in claim 1 and R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a} and wherein R^{a} is independently selected from H, CN, OH, and C₁₋₆alkyl.

66. A compound of formula (la) or a pharmaceutically salt thereof as recited in claim 2 wherein Q, V, X, Y, Z, m, n, q, r, s, t, R² and R³ have the meanings defined in claim 2 and R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a} and wherein R^{a} is independently selected from H, CN, OH, and C₁₋₆alkyl.

67. A compound of formula (Ib) or a pharmaceutically salt thereof as recited in claim 3 wherein Q, V, X, Y, Z, m, n, q, r, s, t, R² and R³ have the meanings defined in claim 3 and R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a} and wherein R^{a} is independently selected from H, CN, OH, and C₁₋₆alkyl.

68. A compound of formula (Ic) or a pharmaceutically salt thereof as recited in claim 4 wherein Q, V, X, Y, Z, m, n, q, r, s, t, R² and R³ have the meanings defined in claim 4 and R⁴ is independently selected from H. halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl, wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a} and wherein R^{a} is independently selected from H, CN, OH, and C₁₋₆alkyl.

69. A compound of formula (I) or a pharmaceutically acceptable salt, thereof as recited in claim 1 wherein:
Q is selected from C₅₋₁₄aryl or C₅₋₁₄heterocyclyl;
R¹ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl, wherein such substituent are independently selected from:
halogen, CN, NH₂, OH, C₁₋₆alkyl-R^{a}, OC₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂;
R^{a} is selected from H, halogen, CN, NH₂, OH, C₁₋₆alkyl, OC₁₋₆alkyl, OC₁₋₆alkyl-OC₁₋₆alkyl, C(═O)OC₁₋₆alkyl, and OC(═O)C₁₋₆alkyl;
V is O;
X is selected from N, O, S, SO, SO₂, NHSO₂, SO₂NH, NHC(═O), C(═O)NH, and optionally substituted C₁₋₆alkyl, C₂₋₆alkenyl or C₃cycloalkyl wherein such substituent is/are independently selected from R²;
Y is optionally substituted C₁₋₆alkyl wherein such substituent is/are independently selected from R²;
Z is selected from N, and optionally substituted C₁₋₆alkyl wherein such substituent is/are independently selected from R²;
k is 0, 1, 2, or 3;
m is 0, 1, 2, or 3;
n is 0, 1, or 2;
q is 0;
r is 1;
s is 1;
t is 0, 1 or 2
R² is independently selected from H, and optionally substituted C₁₋₆alkyl;
R³is independently selected from H, halogen, CN, C₁₋₆alkyl-R^{a}, and C(═O)C₅₋₆heterocyclyl-R^{a}.
R⁴ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from:
halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂NH(C₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂.

70. A compound of formula (Ia) or a pharmaceutically acceptable salt, thereof as recited in claim 2 wherein:
Q is selected from C₅₋₁₄aryl or C₅₋₁₄heterocyclyl;
R¹ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from:
halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO_{NHC}₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂;
R^{a} is selected from H, halogen, CN, NH₂, OH, C₁₋₆alkyl, OC₁₋₆alkyl, OC₁₋₆alkyl-OC₁₋₆alkyl, C(═O)OC₁₋₆alkyl, and OC(═O)C₁₋₆alkyl;
V is O;
X is selected from N, O, S, SO, SO₂, NHSO₂, SO₂NH, NHC(═O), C(═O)NH, and optionally substituted C₁₋₆alkyl, C₂₋₆alkenyl or C₃cycloalkyl wherein such substituent is/are independently selected from R²;
Y is optionally substituted C₁₋₆alkyl wherein such substituent is/are independently selected from R²;
Z is selected from N, and optionally substituted C₁₋₆alkyl wherein such substituent is/are independently selected from R²;
k is 0, 1, 2, or 3;
m is 0, 1, 2, or 3;
n is 0, 1, or 2;
q is 0;
r is 1;
s is 1;
t is 0, 1 or 2
R² is independently selected from H, and optionally substituted C₁₋₆alkyl;
R³ is independently selected from H, halogen, CN, C₁₋₆alkyl-R^{a}, and C(═O)C₅₋₆heterocyclyl-R^{a}.
R⁴ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from:
halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂.

71. A compound of formula (Ib) or a pharmaceutically acceptable salt, thereof as recited in claim 3 wherein:
Q is selected from C₅₋₁₄aryl or C₅₋₁₄heterocyclyl;
R¹ is independently selected from H, halogen, optionally substituted C₁₋₆alkyl, optionally substituted C₂₋₆alkenyl, optionally substituted C₂₋₆allynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from:
halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂;
R^{a} is selected from H, halogen, CN, NH₂, OH, C₁₋₆alkyl, OC₁₋₆alkyl, OC₁₋₆alkyl-OC₁₋₆alkyl, C(═O)OC₁₋₆alkyl, and OC(═O)C₁₋₆alkyl;
V is O;
X is selected from N, O, S, SO, SO₂, NHSO₂, SO₂NH, NHC(═O), C(═O)NH, and optionally substituted C₁₋₆alkyl, C₂₋₆alkenyl or C₃cycloalkyl wherein such substituent is/are independently selected from R²;
Y is optionally substituted C₁₋₆alkyl wherein such substituent is/are independently selected from R²;
Z is selected from N, and optionally substituted C₁₋₆alkyl wherein such substituent is/are independently selected from R²;
k is 0, 1, 2, or 3;
m is 0, 1, 2, or 3;
n is 0, 1, or 2;
q is 0;
r is 1;
s is 1;
t is 0, or 1;
R² is independently selected from H, and optionally substituted C₁₋₆alkyl;
R³ is independently selected from H, halogen, CN, C₁₋₆alkyl-R^{a}, and C(═O)C₅₋₆heterocyclyl-R^{a}.
R⁴ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from:
halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂.

72. A compound of formula (Ic) or a pharmaceutically acceptable salt, thereof as recited in claim 4 wherein:
Q is selected from C₅₋₁₄aryl or C₅₋₁₄heterocyclyl;
R¹ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from:
halogen, CN, NH₂, OH, C₁₋₆alkyl-R^{a}, OC₁₋₆alkyl-R^{a}, C(═O)C₁₋₆alkyl-R^{a}, C(═O)OC₁₋₆ alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₋₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂C₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂;
R^{a} is selected from H, halogen, CN, NH₂, OH, C₁₋₆alkyl, OC₁₋₆alkyl, OC₁₋₆alkyl-OC₁₋₆alkyl, C(═O)OC₁₋₆alkyl, and OC(═O)C₁₋₆alkyl;
V is O;
X is selected from N, O, S, SO, SO₂, NHSO₂, SO₂NH, NHC(═O), C(═O)NH, and optionally substituted C₁₋₆alkyl, C₂₋₆alkenyl or C₃cycloalkyl wherein such substituent is/are independently selected from R²;
Y is optionally substituted C₁₋₆alkyl wherein such substituent is/are independently selected from R²;
Z is selected from N, and optionally substituted C₁₋₆alkyl wherein such substituent is/are independently selected from R²;
k is 0, 1, 2, or 3;
m is 0, 1, 2, or 3;
n is 0, 1, or 2;
q is 0;
r is 1;
s is 1;
t is 0, 1 or 2
R² is independently selected from H, and optionally substituted C₁₋₆alkyl;
R³ is independently selected from H, halogen, CN, C₁₋₆alkyl-R^{a}, and C(═O)C₅₋₆heterocyclyl-R^{a}.
R⁴ is independently selected from H, halogen, optionally substituted C₁₋₆alkyl, optionally substituted C₂₋₆alkenyl, optionally substituted C₂₋₆alkynyl, optionally substituted C₃₋₁₂cycloalkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-₅₋₁₀heterocyclyl, wherein such substituent are independently selected from:
halogen, CN, NH₂, OH, C₁₋₆alkyl-R^{a}, OC₁₋₆alkyl-R^{a}, C(═O)C₁₋₆alkyl-R^{a}, C(═O)OC₁₋₆alkyl-R^{a}, C(═O)NH₂, C(═O)NHC₁₋₆alkyl-R^{a}, C(═O)N(C₁₋₆alkyl)2-R^{a}, SO₂C₁₋₆alkyl-R^{a}, SO₂NHC₁₋₆alkyl-R^{a}, SO₂N(C₁₋₆alkyl)2-R^{a}, NH(C₁₆alkyl)-R^{a}, N(C₁₋₆alkyl)₂-R^{a}, NHC(═O)C₁₋₆alkyl, NHC(═O)C₁₋₆alkyl-R^{a}, NC(═O)(C₁₋₆alkyl)₂, C₅₋₆aryl-R^{a}, OC₅₋₆aryl-R^{a}, C(═O)C₅₋₆aryl-R^{a}, C(═O)OC₅₋₆aryl-R^{a}, C(═O)NH₂, C(═O)NHC₅₋₆aryl-R^{a}, C(═O)N(C₅₋₆aryl)2-R^{a}, SO₂C₅₋₆aryl-R^{a}, SO₂NHC₅₋₆aryl-R^{a}, SO₂N(C₅₋₆aryl)2-R^{a}, NH(C₅₋₆aryl)-R^{a}, N(C₅₋₆aryl)2-R^{a}, NHC(═O)C₅₋₆aryl, NC(═O)(C₅₋₆aryl)₂, C₅₋₆heterocyclyl-R^{a}, OC₅₋₆heterocyclyl-R^{a}, C(═O)C₅₋₆heterocyclyl-R^{a}, C(═O)OC₅₋₆heterocyclyl-R^{a}, C(═O)NH₂, C(═O)NHC₅₋₆heterocyclyl-R^{a}, C(═O)N(C₅₋₆heterocyclyl)₂-R^{a}, SO₂_{C}₅₋₆heterocyclyl-R^{a}, SO₂NHC₅₋₆heterocyclyl-R^{a}, SO₂N(C₅₋₆heterocyclyl)₂-R^{a}, NH(C₅₋₆heterocyclyl)-R^{a}, N(C₅₋₆heterocyclyl)₂-R^{a}, NHC(═O)C₅₋₆heterocyclyl, NC(═O)(C₅₋₆heterocyclyl)₂.

73. A compound of formula (I) or a pharmaceutically acceptable salt, thereof as recited in claim 1 wherein:
Q is C₅₋₁₄aryl;
R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};
R^{a} is selected from H, CN, OH, and C₁₋₆alkyl;
V is O;
Y is unsubstituted C₁₋₆alkyl;
Z is selected from N, and unsubstituted C₁₋₆alkyl;
k is 0, 1, or 2;
m is 0, 1, or 2;
n is 0, 1, or 2;
q is 0;
r is 1;
s is 1;
t is 0, or 1;
R³ is independently selected from H, halogen, or C₁₋₆alkyl-R^{a};
R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

74. A compound of formula (Ia) or a pharmaceutically acceptable salt, thereof as recited in claim 2 wherein:
Q is C₅₋₁₄aryl;
R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};
R^{a} is selected from H, CN, OH, and C₁₋₆alkyl;
V is O;
Y is unsubstituted C₁₋₆alkyl;
Z is selected from N, and unsubstituted C₁₋₆alkyl;
k is 0, 1, or 2;
m is 0, 1, or 2;
n is 0, 1, or 2;
q is 0;
r is 1;
s is 1;
t is 0, or 1;
R³ is independently selected from H, halogen, or C₁₋₆alkyl-R^{a};
R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

75. A compound of formula (Ib) or a pharmaceutically acceptable salt, thereof as recited in claim 3 wherein:
Q is C₅₋₁₄aryl;
R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};
R^{a} is selected from H, CN, OH, and C₁₋₆alkyl;
V is O;
Y is unsubstituted C₁₋₆alkyl;
Z is selected from N, and unsubstituted C₁₋₆alkyl;
k is 0, 1, or 2;
m is 0, 1, or 2;
n is 0, 1, or 2;
q is 0;
r is 1;
s is 1;
t is 0, or 1;
R³ is independently selected from H, halogen, or C₁₋₆alkyl-R^{a};
R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

76. A compound of formula (Ic) or a pharmaceutically acceptable salt, thereof as recited in claim 4 wherein:
Q is C₅₋₁₄aryl;
R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};
R^{a} is selected from H, CN, OH, and C₁₋₆alkyl;
V is O;
Y is unsubstituted C₁₋₆alkyl;
Z is selected from N, and unsubstituted C₁₋₆alkyl;
k is 0, 1, or 2;
m is 0, 1, or 2;
n is 0, 1, or 2;
q is 0;
r is 1;
s is 1;
t is 0, or 1;
R³ is independently selected from H, halogen, or C₁₋₆alkyl-R^{a};
R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

77. A compound of formula (I) or a pharmaceutically acceptable salt, thereof as recited in claim 1 wherein:
Q is C₅₋₁₀aryl;
R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};
R^{a} is selected from H, CN, OH, and C₁₋₆alkyl;
V is O;
Y is unsubstituted C₁₋₆alkyl;
Z is selected from N, and unsubstituted C₁₋₆alkyl
k is 0, 1, or 2;
m is 0, 1, or 2;
n is 0, 1, or 2;
q is 0;
r is 1;
s is 1;
t is 0, or 1;
R³ is independently selected from H, halogen, or C₁₋₆alkyl-R^{a};
R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

78. A compound of formula (Ia) or a pharmaceutically acceptable salt, thereof as recited in claim 2 wherein:
Q is C₅₋₁₀aryl;
R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};
R^{a} is selected from H, CN, OH, and C₁₋₆alkyl;
V is O;
Y is unsubstituted C₁₋₆alkyl;
Z is selected from N, and unsubstituted C₁₋₆alkyl
k is 0, 1, or 2;
m is 0, 1, or 2;
n is 0, 1, or 2;
q is 0;
r is 1;
s is 1;
t is 0, or 1;
R³ is independently selected from H, halogen, or C₁₋₆alkyl-R^{a};
R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

79. A compound of formula (Ib) or a pharmaceutically acceptable salt, thereof as recited in claim 3 wherein:
Q is C₅₋₁₀aryl;
R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};
R^{a} is selected from H, CN, OH, and C₁₋₆alkyl;
V is O;
Y is unsubstituted C₁₋₆alkyl;
Z is selected from N, and unsubstituted C₁₋₆alkyl;
k is 0, 1, or 2;
m is 0, 1, or 2;
n is 0, 1, or 2;
q is 0;
r is 1;
s is 1;
t is 0, or 1;
R³ is independently selected from H, halogen, or C₁₋₆alkyl-R^{a};
R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

80. A compound of formula (Ic) or a pharmaceutically acceptable salt, thereof as recited in claim 4 wherein:
Q is C₅₋₁₀aryl;
R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};
R^{a} is selected from H, CN, OH, and C₁₋₆alkyl;
V is O;
Y is unsubstituted C₁₋₆alkyl;
Z is selected from N, and unsubstituted C₁₋₆alkyl;
k is 0, 1, or 2;
m is 0, 1, or 2;
n is 0, 1, or 2;
q is 0;
r is 1;
s is 1;
t is 0, or 1;
R³ is independently selected from H, halogen, or C₁₋₆alkyl-R^{a};
R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

81. A compound of formula (I) or a pharmaceutically acceptable salt, thereof as recited in claim 1 wherein:
Q is phenyl;
R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};
R^{a} is selected from H, CN, OH, and C₁₋₆alkyl;
V is O;
Y is unsubstituted C₁₋₆alkyl;
Z is selected from N, and unsubstituted C₁₋₆alkyl;
m is 0, 1, or 2;
n is 0, 1, or 2;
q is 0;
r is 1;
s is 1;
t is 0, or 1;
R³ is independently selected from H, halogen, or C₁₋₆alkyl-R^{a}.
R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

82. A compound of formula (Ia) or a pharmaceutically acceptable salt, thereof as recited in claim 2 wherein:
Q is phenyl;
R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};
R^{a} is selected from H, CN, OH, and C₁₋₆alkyl;
V is O;
Y is unsubstituted C₁₋₆alkyl;
Z is selected from N, and unsubstituted C₁₋₆alkyl;
m is 0, 1, or 2;
n is 0, 1, or 2;
q is 0;
r is 1;
s is 1;
t is 0, or 1;
R³ is independently selected from H, halogen, or C₁₋₆alkyl-R^{a}.
R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

83. A compound of formula (Ib) or a pharmaceutically acceptable salt, thereof as recited in claim 3 wherein:
Q is phenyl;
R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};
R^{a} is selected from H, CN, OH, and C₁₋₆alkyl;
V is O;
Y is unsubstituted C₁₋₆alkyl;
Z is selected from N, and unsubstituted C₁₋₆alkyl;
m is 0, 1, or 2;
n is 0, 1, or 2;
q is 0;
r is 1;
s is 1;
t is 0, or 1;
R³ is independently selected from H, halogen, or C₁₋₆alkyl-R^{a}.
R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

84. A compound of formula (Ic) or a pharmaceutically acceptable salt, thereof as recited in claim 4 wherein:
Q is phenyl;
R¹ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a};
R^{a} is selected from H, CN, OH, and C₁₋₆alkyl;
V is O;
Y is unsubstituted C₁₋₆alkyl;
Z is selected from N, and unsubstituted C₁₋₆alkyl;
m is 0, 1, or 2;
n is 0, 1, or 2;
q is 0;
r is 1;
s is 1;
t is 0, or 1;
R³ is independently selected from H, halogen, or C₁₋₆alkyl-R^{a}.
R⁴ is independently selected from H, halogen, optionally substituted C₁₋₆alkyl, optionally substituted C₅₋₁₀aryl, optionally substituted C₁₋₆alkyl-C₅₋₁₀aryl, optionally substituted ₅₋₁₀heterocyclyl or optionally substituted C₁₋₆alkyl-C₅₋₁₀heterocyclyl wherein such substituent are independently selected from: C₁₋₆alkyl-R^{a}, OC₁₋₆alkyl-R^{a}, C(═O)OC₁₋₆alkyl-R^{a}, and C₅₋₆heterocyclyl-R^{a}.

85. A compound of formula (I) selected from:
N˜3˜-(1-naphthylmethyl)pyridine-2,3-diamine;
6-[2-(1H-indol-6-yl)ethyl]pyridin-2-amine;
6-[2-(2-naphthyl)ethyl]pyridin-2-amine;
6-[2-(3-chloro-1H-indol-6-yl)ethyl]pyridin-2-amine;
N˜3˜-(2-naphthylmethyl)pyridine-2,3-diamine;
N˜3˜-(1,1′-biphenyl-4-ylmethyl)pyridine-2,3-diamine;
N˜3˜-(1,1′-biphenyl-3-ylmethyl)pyridine-2,3-diamine;
6-[2-(3,8-dimethoxy-2-naphthyl)ethyl]pyridin-2-amine;
N˜3˜-benzyl-6-phenoxypyridine-2,3-diamine;
N˜3˜-[(3,8-dimethoxy-2-naphthyl)methyl]pyridine-2,3-diamine;
N˜3˜-(3-pyridin-3-ylbenzyl)pyridine-2,3-diamine;
6-[2-(3-pyridin-3-ylphenyl)ethyl]pyridin-2-amine;
N˜3˜-(1-biphenylen-2-ylethyl)pyridine-2,3-diamine;
N˜3˜-[3-(benzyloxy)benzyl]pyridine-2,3-diamine;
N˜3˜-(1,1′-biphenyl-3-ylmethyl)-6-methylpyridine-2,3-diamine;
N˜3˜-(2-phenylethyl)pyridine-2,3-diamine;
6-methyl-N˜3˜-(3-pyridin-3-ylbenzyl)pyridine-2,3-diamine;
N˜3˜-{[4′-(3-methyl-1H-pyrazol-5-yl)-1,1′-biphenyl-3-yl]methyl}pyridine-2,3-diamine;
N˜3˜-[3-(1H-indol-6-yl)benzyl]pyridine-2,3-diamine;
ethyl 3′-{[(2-aminopyridin-3-yl)amino]methyl}-1,1′-biphenyl-3-carboxylate;
(3′-{[(2-aminopyridin-3-yl)amino]methyl}-1,1′-biphenyl-3-yl)acetonitrile;
N˜3˜-(1H-indol-6-ylmethyl)-6-methylpyridine-2,3-diamine;
N˜3˜-{3-[5-(1H-pyrazol-5-yl)thien-2-yl]benzyl}pyridine-2,3-diamine;
N˜3˜-(4-methoxy-2,3-dimethylbenzyl)pyridine-2,3-diamine;
2-(3′-{[(2-aminopyridin-3-yl)amino]methyl}-1,1′-biphenyl-3-yl)ethanol;
(3′-{[(2-aminopyridin-3-yl)amino]methyl}-1,1′-biphenyl-3-yl)methanol;
N˜3˜-{[5-(4-fluorophenyl)pyridin-3-yl]methyl}-6-methylpyridine-2,3-diamine;
N˜3˜-(3-butoxybenzyl)pyridine-2,3-diamine;
N˜3˜-(5-bromo-2-ethoxybenzyl)pyridine-2,3-diamine;
N˜3˜-[3-(cyclopentyloxy)benzyl]pyridine-2,3-diamine;
N˜3˜-[(9-ethyl-9H-carbazo1-3-yl)methyl]pyridine-2,3-diamine;
N˜3˜-[3-(1H-indol-6-yl)benzyl]-6-methylpyridine-2,3-diamine;
N˜3˜-(5-bromo-2-methoxybenzyl)-6-methylpyridine-2,3-diamine;
6-methyl-N˜3˜-[(3-phenyl-1,3-dihydro-2, 1-benzisoxazol-5-yl)methyl]pyridine-2,3-diamine;
N˜3˜-[5-(1H-indol-6-yl)-2-methoxybenzyl]-6-methylpyridine-2,3-diamine;
N˜3˜-[2-(alkyloxy)-5-bromobenzyl]pyridine-2,3-diamine;
N˜3˜-[2-(benzyloxy)-5-bromobenzyl]pyridine-2,3-diamine;
N˜3˜-[5-bromo-2-(prop-2-ynyloxy)benzyl]pyridine-2,3-diamine;
(3′-{[(2-amino-6-methylpyridin-3-yl)amino]methyl}-4′-methoxy-1,1′-biphenyl-3-yl)acetonitrile;
N˜3˜-[(3′-amino-4-methoxy-1,1′-biphenyl-3-yl)methyl]-6-methylpyridine-2,3-diamine;
N-(3′-{[(2-amino-6-methylpyridin-3-yl)amino]methyl}-4′-methoxy-1,1′-biphenyl-3-yl)acetamide;
N˜3˜-(5-isoquinolin-4-yl-2-methoxybenzyl)-6-methylpyridine-2,3-diamine;
6-{2-[3-(1H-indol-6-yl)phenyl]ethyl}pyridin-2-amine;
N˜3˜-(3-isoquinolin-4-ylbenzyl)-6-methylpyridine-2,3-diamine;
N˜3˜-[(3′-amino-1,1′-biphenyl-3-yl)methyl]-6-methylpyridine-2,3-diamine;
N˜3˜-[2-(benzyloxy)-5-(1H-indol-6-yl)benzyl]pyridine-2,3-diamine;
5-bromo-N˜3˜-[3-(1H-indol-6-yl)benzyl]pyridine-2,3-diamine;
N˜3˜-[(3′,4-dimethoxy-1,1′-biphenyl-3-yl)methyl]-6-methylpyridine-2,3-diamine;
N˜3˜-[5-bromo-2-(pyridin-2-ylmethoxy)benzyl]pyridine-2,3-diamine;
N˜3˜-[(3′-methoxy-1,1′-biphenyl-3-yl)methyl]-6-methylpyridine-2,3-diamine;
N˜3˜-[5-(1H-indol-6-yl)-2-(pyridin-2-ylmethoxy)benzyl]pyridine-2,3-diamine;
N˜3-˜[2-(benzyloxy)-5-pyridin-3-ylbenzyl]pyridine-2,3-diamine;
5-bromo-N˜3˜-[(3′-methoxy-1,1′-biphenyl-3-yl)methyl]pyridine-2,3-diamine;
3-{[(2-aminopyridin-3-yl)amino]methyl}-3′-methoxy-1,1′-biphenyl-4-ol;
N˜3˜-[3-(2-furyl)benzyl]-6-methylpyridine-2,3-diamine;
N˜3˜-[5-(2-furyl)-2-methoxybenzyl]-6-methylpyridine-2,3-diamine;
N˜3˜-[2-(benzyloxy)-5-tert-butylbenzyl]pyridine-2,3-diamine;
N˜3˜-{[3′-methoxy-4-(pyridin-2-ylmethoxy)-1,1′-biphenyl-3-yl]methyl}pyridine-2,3-diamine;
6-chloro-N˜3˜-[(3′-methoxy-1,1′-biphenyl-3-yl)methyl]pyridine-2,3-diamine;
5-chloro-N˜3˜-[(3′-methoxy-1,1′-biphenyl-3-yl)methyl]pyridine-2,3-diamine;
N˜3˜-{[4-(benzyloxy)-3′-methoxy-1,1′-biphenyl-3-yl]methyl}pyridine-2,3-diamine;
N˜3˜-[5-bromo-2-(pyridin-3-ylmethoxy)benzyl]pyridine-2,3-diamine;
N˜3˜-[5-(1H-indol-6-yl)-2-(pyridin-3-ylmethoxy)benzyl]pyridine-2,3-diamine;
N˜3˜-[5-(1H-indol-6-yl)-2-(pyridin-4-ylmethoxy)benzyl]pyridine-2,3-diamine;
N˜3˜-[3-(6-chloropyridin-3-yl)benzyl]pyridine-2,3-diamine;
N˜3˜-[(3′,4′-dimethoxy-1,1′-biphenyl-3-yl)methyl]pyridine-2,3-diamine;
5-chloro-N˜3˜-(3-pyridin-3-ylbenzyl)pyridine-2,3-diamine;
N˜3˜-[3-(5-methoxypyridin-3-yl)benzyl]pyridine-2,3-diamine;
2-[2-(6-aminopyridin-2-yl)ethyl]-4-(1H-indol-6-yl)phenol;
6-{2-[5-(1H-indol-6-yl)-2-(pyridin-2-ylmethoxy)phenyl]ethyl}pyridin-2-amine;
N˜3˜-[3-(2-furyl)benzyl]pyridine-2,3-diamine;
N˜3˜-[2-[2-(dimethylamino)ethoxy]-5-(1H-indol-6-yl)benzyl]pyridine-2,3-diamine;
N˜3˜-[5-pyridin-3-yl-2-(2-pyrrolidin-1-ylethoxy)benzyl]pyridine-2,3-diamine;
N˜3˜-[5-(1H-indol-6-yl)-2-(2-pyrrolidin-1-ylethoxy)benzyl]pyridine-2,3-diamine;
N˜3˜-(3-pyrimidin-5-ylbenzyl)pyridine-2,3-diamine;
N˜3˜-(5-bromo-2-isobutoxybenzyl)pyridine-2,3-diamine;
6-{2-[3-(5-methoxypyridin-3-yl)phenyl]ethyl}pyridin-2-amine;
6-[2-(3′-methoxy-1,1′-biphenyl-3-yl)ethyl]pyridin-2-amine;
6-{2-[3-(2-furyl)phenyl]ethyl}pyridin-2-amine;
N˜3˜-[3-(2-methyl-1,3-benzothiazol-5-yl)benzyl]pyridine-2,3-diamine;
N˜3˜-[3-(6-methoxypyridin-3-yl)benzyl]pyridine-2,3-diamine;
N˜3˜-[(3′-amino-1,1′-biphenyl-3-yl)methyl]pyridine-2,3-diamine;
6-[2-(3′,4′-dimethoxy-1,1′-biphenyl-3-yl)ethyl]pyridin-2-amine;
N˜3˜-(3-bromobenzyl)-6-(morpholin-4-ylcarbonyl)pyridine-2,3-diamine;
N-(3′-{[(2-aminopyridin-3-yl)amino]methyl}-1,1′-biphenyl-3-yl)propanamide;
N-(3′-{[(2-aminopyridin-3-yl)amino]methyl}-1,1′-biphenyl-3-yl)butanamide;
methyl 3-[(3′-{[(2-aminopyridin-3-yl)amino]methyl}-1,1′-biphenyl-3-yl)amino]-3-oxopropanoate;
2-[(3′-{[(2-aminopyridin-3-yl)amino]methyl}-1,1′-biphenyl-3-yl)amino]-2-oxoethyl acetate;
N-(3′-{[(2-aminopyridin-3-yl)amino]methyl}-1,1′-biphenyl-3-yl)-2-(2-methoxyethoxy)acetamide;
N˜3˜-[3-(5-propoxypyridin-3-yl)benzyl]pyridine-2,3-diamine;
{6-amino-5-[(3-bromobenzyl)amino]pyridin-2-yl}methanol;
{6-amino-5-[(3-pyridin-3-ylbenzyl)amino]pyridin-2-yl} methanol;
2-amino-6-[2-(3′-methoxy-1,1′-biphenyl-3-yl)ethyl]nicotinonitrile;
6-(2-Biphenyl-3-yl-ethyl)-3-methyl-pyridin-2-ylamine;
6-[2-(3′-methoxy-biphenyl-3-yl)-ethyl]-3-methylpyridin-2-ylamine;
6-(2-biphenyl-3-ylethyl)-5-fluoro-pyridin-2-ylamine;
5-fluoro-6-[2-(3′-methoxy-biphenyl-3-yl)-ethyl]-pyridin-2-ylamine.

86. A compound of formula (I) according to claim 1 or a pharmaceutically acceptable salt thereof for use as a medicament.

87. The use of a compound of formula (I) according to claim 1 or a pharmaceutically acceptable salt thereof, in the manufacture of a medicament for the treatment or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia.

88. A method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia comprising administering to a human a therapeutically effective amount of a compound of formula (I) or a pharmaceutically acceptable salt, thereof as defined in claim 1.

89. A method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia comprising administering to a human a therapeutically effective amount of a compound of formula (I) or a pharmaceutically acceptable salt, thereof as defined in claim 1.

90. A method of treating Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer Disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia by administering to a human a compound of formula (I) or a pharmaceutically acceptable salt, thereof as defined in claim 1 and a cognitive and/or memory enhancing agent.

91. A method of treating Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia by administering to a human a compound of formula (I) or a pharmaceutically acceptable salt, thereof as defined in claim 1 and a choline esterase inhibitor.

92. A pharmaceutical composition comprising a compound of formula (I) or a pharmaceutically acceptable salt thereof as defined in claim 1 together with at least one pharmaceutically acceptable carrier, diluent or excipent.

93. A compound according to Formula (E): 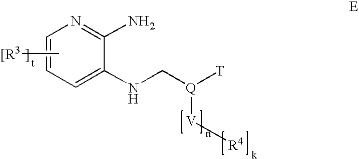
wherein T is a coupling partner, R³, R⁴, Q, V, t, n and k are as defined in claim 1 for Formula (I).

94. Use of a compound of claim 93 as a chemical intermediate in the preparation of a compound of formula I, according to claim 1, wherein Z is N, s is 1, Y is C₁₋₆alkyl, r is 1 and q is 0.

95. A compound according to Formula (F): 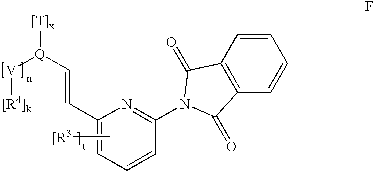
wherein T is a coupling partner, x is 0 or 1, R³, R⁴, Q, V, t, n and k are as defined in claim 1 for Formula (I).

96. A compound according to Formula (G): 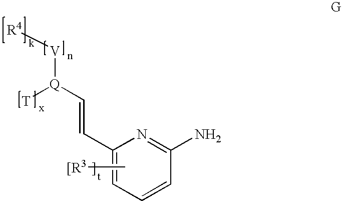
wherein T is a coupling partner, x is 0 or 1, R³, R⁴, Q, V, t, n and k are as defined in claim 1 for Formula (I).

97. A compound according to Formula (D): 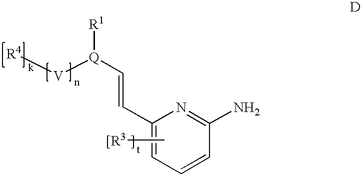
wherein R¹, R³, R⁴, Q, V, t, n and k are as defined in claim 1 for Formula (I).

98. Use of a compound of claim 95 as a chemical intermediate in the preparation of a compound of formula I, according to claim 1, wherein Z is C₁-alkyl, s is 1, Y is C₁₋₆alkyl, r is 1 and q is 0.

99. Use of a compound of claim 96 as a chemical intermediate in the preparation of a compound of formula I, according to claim 1, wherein Z is C₁-alkyl, s is 1, Y is C₁₋₆alkyl, r is 1 and q is 0.

100. Use of a compound of claim 97 as a chemical intermediate in the preparation of a compound of formula I, according to claim 1, wherein Z is C₁-alkyl, s is 1, Y is C₁₋₆alkyl, r is 1 and q is 0.
